(19) United States
(12) Patent Application Publication
(10) Pub. No.: US 2004/0048248 A1
(43) Pub. Date: Mar. 11, 2004

(54) Endozepine-like polypeptides and polynucleotides encoding same

(76) Inventors: Sudhirdas K. Prayaga, O'Fallon, MO (US); Richard A. Shimkets, Guilford, CT (US); Kumud Majumder, Stamford, CT (US); Andrew Eisen, Rockville, MD (US); Corine A.M. Vernet, Branford, CT (US); Steven K. Spaderna, Berlin, CT (US); Jason Baumgartner, New Haven, CT (US); Linda Gorman, Branford, CT (US); Vladimir Gusev, Madison, CT (US); Muralidhara Padigaru, Branford, CT (US); Meera Patturajan, Branford, CT (US); Velizar Tchernev, Branford, CT (US); Li Li, Branford, CT (US)

Correspondence Address: Ivor R. Elrifi, MINTZ, LEVIN, COHN, FERRIS,, GLOVSKY AND POPEO, P.C., One Financial Center, Boston, MA, 02111, US

(21) Appl. No.: 10/083,919

(22) Filed: Feb. 27, 2002

Related U.S. Application Data
(63) Continuation-in-part of application No. 09/679,740, filed on Oct. 5, 2000.
(60) Provisional application No. 60/157,786, filed on Oct. 5, 1999.
(60) Provisional application No. 60/164,164, filed on Nov. 9, 1999.
(60) Provisional application No. 60/174,505, filed on Jan. 4, 2000.
(60) Provisional application No. 60/183,859, filed on Feb. 22, 2000.
(60) Provisional application No. 60/190,740, filed on Mar. 20, 2000.
(60) Provisional application No. 60/191,133, filed on Mar. 22, 2000.
(60) Provisional application No. 60/206,006, filed on May 19, 2000.
(60) Provisional application No. 60/215,684, filed on Jun. 30, 2000.
(60) Provisional application No. 60/219,490, filed on Jul. 20, 2000.
(60) Provisional application No. 60/227,072, filed on Aug. 22, 2000.
(60) Provisional application No. 60/271,909, filed on Feb. 27, 2001.

(51) Int. Cl. A61K 48/00; C12Q 1/68; C07H 21/04; C12N 9/02; C12P 21/02; C12N 5/06

(52) U.S. Cl. 435/6; 435/69.1; 435/189; 435/320.1; 435/325; 514/44; 536/23.2

(57) ABSTRACT

Disclosed herein are novel human nucleic acid sequences which encode endozepine-like polypeptides. Also disclosed are polypeptides encoded by these nucleic acid sequences, and antibodies which immunospecifically-bind to the polypeptide, as well as derivatives, variants, mutants, or fragments of the aforementioned polypeptide, polynucleotide, or antibody. The invention further discloses therapeutic, diagnostic and research methods for diagnosis, treatment, and prevention of disorders involving this novel human endozepine-like nucleic acid and protein.

### RELATED APPLICATIONS

This application claims priority to U.S. S No. 60/157,786, filed Oct. 5, 1999; U.S. S No. 60/164,164, filed Nov. 9, 1999; U.S. S No. 60/174,505, filed Jan. 4, 2000; U.S. S No. 60/183,859, filed Feb. 22, 2000; U.S. S No. 60/190,740, filed Mar. 20, 2000, U.S. S No. 60/191,133, filed Mar. 22, 2000, U.S. S No. 60/206,006, filed May 19, 2000, U.S. S No. 60/215,684, filed Jun. 30, 2000, U.S. S No. 60/219,490, filed Jul. 20, 2000, U.S. S No. 60/227,072, filed Aug. 22, 2000, U.S. Ser. No. 09/679,460, filed Oct. 4, 2000, U.S. Ser. No. 09/679,740, filed Oct. 5, 2000, and U.S. S No. 60/271,909, filed Feb. 27, 2001. The contents of these applications are incorporated herein by reference in their entireties.

### FIELD OF THE INVENTION

The invention generally relates to nucleic acids and polypeptides encoded therefrom. More specifically, the invention relates to nucleic acids encoding endozepine-like polypeptides, as well as vectors, host cells, antibodies, and recombinant methods for producing these nucleic acids and polypeptides.

### BACKGROUND OF THE INVENTION

In the developed world there are over 250 million individuals who suffer from disorders associated with nutritional excess. Obesity, Type II diabetes, and the metabolic syndrome X have reached epidemic proportions. The problems associated with these disorders such as hyperlipidemia, hypertension, vascular disease, stroke and end-organ damage exact a huge financial burden to afflicted individuals and society at large. Thus, new pharmacologic approaches to regulate energy metabolism would be of significant medical benefit. The present invention contains a novel family of endozepine polypeptides that offer new therapeutic opportunities to regulate metabolism through their modulation of insulin secretion, insulin sensitivity, glucose and fatty acid utilization and other endocrine functions.

Endozepines are a family of proteins whose members have been reported to have diverse biological effects. These effects can include modulation of gamma-aminobutyric acid (GABA) receptors, insulin homeostasis, and regulation of mitochondrial steriodogenesis. Modulation of GABA receptors, which are present in brain, is thought to modulate pathological anxiety.

Members of the endozepine family include diazepam binding inhibitor (DBI). DBI, or a derivative of DBI, is thought to down-regulate the effects of GABA. DBI is also reported to inhibit both the early and the late phases of glucose-induced insulin release from the isolated perfused rat pancreas.

Several mammalian DBI polypeptides have been described. Mammalian DBIs tend to be highly conserved at their carboxy termini. A human DBI polypeptide of approximately 11 kilodaltons (kD) has been described. This polypeptide has been shown to displace β-carbolines and benzodiazepines bound to brain membrane fractions in vitro.

DBI polypeptides have been reported to be present in both brain and non-brain tissue, including gut. It has been proposed that DBI may belong to a new family of gut polypeptides that inhibit glucose-mediated insulin release by hormonal, neurocrine mechanisms, or both.

### SUMMARY OF THE INVENTION

The invention is based in part upon the discovery of novel nucleic acid sequences encoding polypeptides related to known endozepines. Nucleic acids encoding endozepine-like polypeptides and derivatives and fragments thereof, will hereinafter be collectively designated as “ENDOX”.

In one aspect, the invention provides an isolated ENDOX nucleic acid molecule encoding an ENDOX polypeptide that includes a nucleic acid sequence that has identity to the nucleic acid sequence of human endozepine mRNA. In some embodiments, the ENDOX nucleic acid molecule can hybridize under stringent conditions to a nucleic acid sequence complementary to a nucleic acid molecule that includes a protein-coding sequence of an endozepine nucleic acid sequence. The invention also includes an isolated nucleic acid that encodes an ENDOX polypeptide, or a fragment, homolog, analog or derivative thereof. For example, the nucleic acid can encode a polypeptide at least 85% identical to a polypeptide comprising the amino acid sequences of SEQ ID NO:2, 4, 6, 8, 10, 15, 16, 17, 18, 19, 20, 21, 23, 24, 26, 27, 29, 30, 32, 33, 35 to 45 inclusive, 47, 49, 153, 157, 166, 175, 179, 183, 192, and 198. The nucleic acid can be, for example, a genomic DNA fragment or a cDNA molecule that includes the nucleic acid sequence of any of SEQ ID NO: 1, 3, 5, 7, 9, 22, 25, 28, 31, 34, 46, 48, 152, 156, 165, 174, 178, 182, and 191.

Also included in the invention is an oligonucleotide, e.g., an oligonucleotide which includes at least 6 contiguous nucleotides of an ENDOX nucleic acid (e.g., SEQ ID NO: 1, 3, 5, 7, 9, 22, 25, 28, 31, 34, 46, 48, 152, 156, 165, 174, 178, 182, and 191) or a complement of said oligonucleotide.

Also included in the invention are substantially purified ENDOX polypeptides (SEQ ID NO:2, 4, 6, 8, 10, 15, 16, 17, 18, 19, 20, 21, 23, 24, 26, 27, 29, 30, 32, 33, 35 to 45 inclusive, 47, 49, 153, 157, 166, 175, 179, 183, 192, and 198). In some embodiments, the ENDOX polypeptides include an amino acid sequence that is substantially identical to the amino acid sequence of human endozepine polypeptide.

The invention also features antibodies that immunoselectively-binds to ENDOX polypeptides.

In another aspect, the invention includes pharmaceutical compositions which include therapeutically- or prophylactically-effective amounts of a therapeutic and a pharmaceutically-acceptable carrier. The therapeutic can be, e.g., an ENDOX nucleic acid, an ENDOX polypeptide, or an antibody specific for an ENDOX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes an ENDOX nucleic acid, under conditions allowing for expression of the ENDOX polypeptide encoded by the DNA. If desired, the ENDOX polypeptide can then be recovered.

In another aspect, the invention includes a method of detecting the presence of an ENDOX polypeptide in a sample. In the method, a sample is contacted with a compound that selectively binds to the polypeptide under conditions allowing for formation of a complex between the polypeptide and the compound. The complex is detected, if present, thereby identifying the ENDOX polypeptide within the sample.

The invention also includes methods to identify specific cell or tissue types based on their expression of ENDOX.

Also included in the invention is a method of detecting the presence of an ENDOX nucleic acid molecule in a sample by contacting the sample with an ENDOX nucleic acid probe or primer, and detecting whether the nucleic acid probe or primer bound to an ENDOX nucleic acid molecule in the sample.

In a further aspect, the invention provides a method for modulating the activity of an ENDOX polypeptide by contacting a cell sample that includes the ENDOX polypeptide with a compound that binds to the ENDOX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a Therapeutic in the manufacture of a medicament for treating or preventing disorders or syndromes including, e.g., metabolic disorders, diabetes, obesity, infectious disease, anorexia, cancer-associated cachexia, and the various dyslipidemias. The Therapeutic can be, e.g., an ENDOX nucleic acid, an ENDOX polypeptide, or an ENDOX-specific antibody, or biologically-active derivatives or fragments thereof.

The invention further includes a method for screening for a modulator of disorders or syndromes including, e.g., metabolic disorders, diabetes, obesity, infectious disease, anorexia, cancer-associated cachexia, and the various dyslipidemias. The method includes contacting a test compound with an ENDOX polypeptide and determining if the test compound binds to said ENDOX polypeptide. Binding of the test compound to the ENDOX polypeptide indicates the test compound is a modulator of activity, or of latency or predisposition to the aforementioned disorders or syndromes.

Also within the scope of the invention is a method for screening for a modulator of activity, or of latency or predisposition to an disorders or syndromes including, e.g., metabolic disorders, diabetes, obesity, infectious disease, anorexia, cancer-associated cachexia, and the various dyslipidemias, by administering a test compound to a test animal at increased risk for the aforementioned disorders or syndromes. The test animal expresses a recombinant polypeptide encoded by an ENDOX nucleic acid. Expression or activity of ENDOX polypeptide is then measured in the test animal, as is expression or activity of the protein in a control animal which recombinantly-expresses ENDOX polypeptide and is not at increased risk for the disorder or syndrome. Next, the expression of ENDOX polypeptide in both the test animal and the control animal is compared. A change in the activity of ENDOX polypeptide in the test animal relative to the control animal indicates the test compound is a modulator of latency of the disorder or syndrome.

In yet another aspect, the invention includes a method for determining the presence of or predisposition to a disease associated with altered levels of an ENDOX polypeptide, an ENDOX nucleic acid, or both, in a subject (e.g., a human subject). The method includes measuring the amount of the ENDOX polypeptide in a test sample from the subject and comparing the amount of the polypeptide in the test sample to the amount of the ENDOX polypeptide present in a control sample. An alteration in the level of the ENDOX polypeptide in the test sample as compared to the control sample indicates the presence of or predisposition to a disease in the subject. Preferably, the predisposition includes, e.g., metabolic disorders, diabetes, obesity, infectious disease, anorexia, cancer-associated cachexia, and the various dyslipidemias. Also, the expression levels of the new endozepines of the invention can be used in a method to screen for various cancers.

In a further aspect, the invention includes a method of treating or preventing a pathological condition associated with a disorder in a mammal by administering to the subject an ENDOX polypeptide, an ENDOX nucleic acid, or an ENDOX-specific antibody to a subject (e.g., a human subject), in an amount sufficient to alleviate or prevent the pathological condition. In preferred embodiments, the disorder, includes, e.g., metabolic disorders, diabetes, obesity, infectious disease, anorexia, cancer-associated cachexia, and the various dyslipidemias.

In a further aspect, the invention includes a method to alter global energy metabolism or weight loss by altering in serum cholesterol, lipids, glucose and insulin.

In yet another aspect, the invention includes a method to modulate weight loss due to specific adipose deposit reduction, muscle mass increases associated with some medical treatments, or modulation of lipid volume in some adipocytes

In yet a further aspect, the invention can be used in methods to influence appetite, absorption of nutrients and the disposition of metabolic substrates in both a positive and negative fashion. The invention can also be used in the treatment of diabetes, metabolic disturbances associated with obesity, the metabolic syndrome X as well as anorexia and wasting disorders associated with chronic diseases and various cancers by modulating metabolism.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

Unless otherwise defined, all technical and scientific terms used herein have the same meaning as commonly understood by one of ordinary skill in the art to which the invention belongs. Although methods and materials similar or equivalent to those described herein can be used in the practice or testing of the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative purposes only, and not intended to be limiting in any manner. Other features and advantages of the invention will be apparent from the following detailed description and claims.

### BRIEF DESCRIPTION OF THE DRAWINGS

FIG. 1 shows 6 photomicrographs deoicting mesenteric adipose deposits in mice in response to treatment.

FIG. 2 shows PCR products of endozepine coding sequences.

### DETAILED DESCRIPTION OF THE INVENTION

The invention is based, in part, upon the discovery of novel nucleic acid sequences that encode polypeptides related to previously described polypeptides in the endozepine protein family. Nucleic acids encoding endozepine-like polypeptides based on the discovered sequences are referred to individually as ENDO1, ENDO1A, ENDO1B, ENDO2, ENDO2A, ENDO2B, ENDO3, ENDO4, ENDO4A, ENDO5, ENDO6, ENDO7, ENDO7A, ENDO8, ENDO9, ENDO10, and ENDO11. The nucleic acids, and their encoded polypeptides, are collectively designated herein as “ENDOX”.

ENDO1

An ENDO1 nucleic acid of the invention comprises nucleic acid sequences shown in SEQ ID NOs: 1, 11, 13, 14, 46, and 48. SEQ ID NOs: 11, 13, and 14 can be combined to provide the nucleotide sequence of SEQ ID NO:1. SEQ ID NO:1 may not be a complete coding sequence as it does not have a starting methioine, and it has no stop codon. SEQ ID NOs: 46 and 48 both encompass nucleotide sequences which encode an amino acid sequence which is also encoded by SEQ ID NO:1. Each of these nucleotide sequences and the amino acids which they encode are described in detail before.

An ENDO1 nucleic acid of the invention includes the nucleic acid sequence shown in Table 1 (SEQ ID NO:11). The sequence is related to an expression sequence tag (EST) from a previously described human cDNA clone, with database accession number AA877351. The AA877351 EST is reported to be similar to a nucleic acid encoding “diazepam binding inhibitor-like 5”.

TABLE 1

| | |
|---|---|
| ACCGCCTCCACCACCCCATGTGCCAAGTGGAGTTCGAGCTGCGCGGCCCTCAAGCAGCTGAAG | (SEQ ID NO:11) |
| GGTCCCGTGAGCGATCAGGAGAAGCTGCTGGTCTACGGCTTGTACAAACAGGCCACCCAGGGC | |
| GACTGCGACATCCCCGGCCCTCCGGCCTCAGACGTGAGAGCCAGGGCCAAGTGGGAGGCTTGG | |
| AGCGCGAACAAAGGGGCGTCCAAGATGGACGCCATGAGGGGCTACGCGGCCAAAGTGGAGGAG | |
| CTGACGAAGAAGGAA | |

The nucleic acid sequence disclosed in Table 1 includes an open reading frame (“ORF”) beginning at position 1. The ORF encodes a polypeptide sequence of 89 amino acid residues. The sequence of this encoded polypeptide is presented in Table 2 (SEQ ID NO:12). The translated protein is related (Identities 62/81; 76%) and Positives=70/81; 86%) to diazepam binding inhibitor-like 5(SWISSPROT-ACC:009035) from mouse. Also, the translated protein is related (Identities=89/89 (100%), Positives=89/89 (100%)) to Homo sapiens endozepine-like protein type 2 mutant (GENBANK-ID:AF229804|acc:AF229804).

TABLE 2

| | |
|---|---|
| TASTTPCAKWSSSCAALKQLKGPVSDQEKLLVYGLYKQATQGDCDIPGPPASDVRARAKWEAW | (SEQ ID NO:12) |
| SANKGASKMDAMRGYAAKVEELTKKE | |

The polypeptide encoded by the ORF present in SEQ ID NO:11 does not contain an amino terminal methionine residue. Therefore, the disclosed nucleic acid sequence may be a portion of an open reading frame encoding a larger gene product. The larger gene product may include, for example, at least a signal peptide that is cleaved during protein processing.

To identify additional sequences encoding an ENDO1 nucleic acid, the disclosed ENDO1 sequence (SEQ ID NO:11) was used to design primers for use in PCR reactions to isolate additional sequences encoding the ENDO1 polypeptide shown in Table 2. The amplified sequences were cloned and sequenced. The sequences of two products were named 18517852-2 (SEQ ID NO: 13) and 118517852-3 (SEQ ID NO:14). These sequences are shown in Tables 3 and 4, respectively.

TABLE 3

| | |
|---|---|
| TCTTCTTCGTCAGCTCCTCCACTTTGGCCGCGTAGCCCCTCATGGCGTCCATCTTGGACGCCCCTTTGTTCGCGCT | (SEQ ID NO:13) |
| CCAAGCCTCCCACTTGGCCCTGGCTCTCACGTCTGAGGCCGGAGGGCCGGGGATGTCGCAGTCGCCCTGGGTGGCC | |
| TGTTTGTACAAGCCGTAGACCAGCAGCTTCTCCTGATCGCTCACGGGACCCTTCAGCTGCTTGAGGGCCGCAAAGC | |
| TCGAACTCCACTTGGCACATGGGGTGGTGGAGGCGGTCCCTGGTGCTAGAAGCTGGAGGTGGAGAGTTGGAGTGGC | |
| TGTTACTACTCGATCTCAGGGGGAGGAGACAGGCACGCGATGTTTGTGTTTTGTCAAGCACAGATTGCAAGCTCGG | |
| GGTCCAGCGTAAACCCCACCATGTTTGGGCTCACACGGCGCATTTTCTGGGGAGGACCAGCCGTCAAAAAGCGTCT | |
| AGGATCCGGAACGCTGCTGTCTGGA | |

TABLE 4

(SEQ ID NO:14)

GCGTCCATCTTGGACGCCCCTTTGTTCGCGCTCCAAGCCTCCCACTTGGC
CCTGGCTCTCACGTCTGAGGCCGGAGGGCCGGGGATGTCGCAGTCGCCCT
GGGTGGCCTGTTTGTACAAGCCGTAGACCAGCAGCTTCTCCTGATCGCTC
ACGGGACCCTTCAGCTGCTTGAGGGCCGCGCAGCTCGAACTCCACTTGGC
ACATGGGGTGGTGGAGGCGGTCCCTGGTGCTAGAAGCTGGAGGTGGAGAG
TTGGAGTGGCTGTTACTACTCGC

The sequences shown in Tables 1, 3, and 4 can be combined to provide the nucleotide sequence shown in Table 5 (SEQ ID NO:1).

TABLE 5

(SEQ ID NO:1)

GAT CGA GTA GTA ACA GCC ACT CCA ACT CTC CAC CTC CAG CTT CTA GCA CCA GGG ACC GCC TCC
ACC ACC CCA TGT GCC AAG TGG AGT TCG AGC TXT GCG GCC CTC AAG CAG CTG AAG GGT CCC GTG
AGC GAT CAG GAG AAG CTG CTG GTC TAC GGC TTG TAC AAA CAG GCC ACC CAG GGC GAC TGC GAC
ATC CCC GGC CCT CCG GCC TCA GAC GTG AGA GCC AGG GCC AAG TGG GAG GCT TGG AGC GCG AAC
AAA GGG GCG TCC AAG ATG GAC GCC ATG AGG GGC TAC GCG GCC AAA GTG GAG GAG CTG ACG AAG
AAG

The nucleotide residue denoted by “X” can be T or G, i.e., in various embodiments an ENDO1 nucleic acid of the invention includes a T at the position denoted by X in the nucleic acid sequence. In other embodiments, an ENDO1 nucleic acid sequence of the invention includes a G at the position denoted by X in the disclosed nucleotide sequence.

The polypeptide encoded by the ORF in SEQ ID NO:1 does not contain an amino terminal methionine residue. Therefore, the disclosed nucleic acid sequence may be a portion of an open reading frame encoding a larger gene product. To identify additional sequences encoding an ENDO1 nucleic acid, nucleic acid database searches were conducted. Two new ENDO1 sequences have now been indentified and are shown in Table 5A. SEQ ID NO:46 was compiled from a previously described human clone with GenBank AccNo AL121672 (SEQ ID NO:46). SEQ ID NO:48 was compiled from a previously described human clone with Genank clone AC025743 (SEQ ID NO:48).

TABLE 5A

| | |
|---|---|
| >AL121672_GENSCAN_predicted_CDS_5_687_bp | |
| ATGGGAGACGCAGGAGCCACGGCGGCCGCGCTTAGGCCTGCTCACAACCTCCGCCCGGCCCCGCCCACAG | (SEQ ID NO:46) |
| CCTCCGCCGCGCACGCGCAGTCCTCACGAACGAGCGCGCCAAGCGCACAGCGCCGCCTTCCGGCAGAGCC | |
| CTCCCACCAGCCCTCAGCACCAGGGACCGCCTCCACCACCCCATGTGCCAAGTGGAGTTCGAGCTGCGCG | |
| GCCCTCAAGCAGCTGAAGGGTCCCGTGAGCGATCAGGAGAAGCTGCTGGTCTACGGCTTGTACAAACAGG | |
| CCACCCAGGGCGACTGCGACATCCCCGGCCCTCCGGCCTCAGACGTGAGAGCCAGGGCCAAGTGGGAGGC | |
| TTGGAGCGCGAACAAAGGGGCGTCCAAGATGGACGCCATGAGGGGCTACGCGGCCAAAGTGGAGGAGCTG | |
| ACGAAGAAGGAAGTGGGGGGCGTGGAGCGCGAACAAAGGGGCGTGCAAGATGGACGCCATGAGGGGCTAC | |
| GCGGCCAAAGTGGAGGAGCTGACGAAGAAGGAAGGGCGTCCAAGATGGACGCCATGAGGGGCTACGCGGC | |
| CAAAGTGGAGGAGCTGACGAAGAAGGAAGTGGGGGGCGTGGAGCGCGAACAAAGGGGCGTCCAAGATGGA | |
| CGCCATGAGGGGCTACGCGGCCAGAGTGAGGAGATGAGGAAGAAGGAGGCTGGCTGA | |
| >AC025743_GENSCAN_predicted_CDS_7_576_bp | |
| ATGGGAGACGCAGGAGCCACGGCGGCCGCGCTTAGGCCTGCTCACAACCTCCGCCCGGCCCCGCCCACAG | (SEQ ID NO:48) |
| CCTCCGCCGCGCACGCCAGTCCTCACGAACGAGCGCGCCAAGCAAGCCGCGCCTTCCGGCAGAGCCCTCC | |
| CACCAGCCCTCAGCTTCTAGCACCAGGGACCGCCTCCACCACCCCATGTGCCAAGTGGAGTTCGAGCTGC | |
| GCGGCCCTCAAGCAGCTGAAGGGTCCCGTGAGCGATCAGGAGAAGCTGCTGGTCTACGGCTTGTACAAAC | |
| AGGCCACCCAGGGCGACTGCGACATCCCCGGCCCTCCGGCCTCAGACGTGAGAGCCAGGGCCAAGTGGGA | |
| GGCTTGGAGCGCGAAAAAAGGGGCGTCCAAGATGGACGCCATGAGGGGCTACGCGGCCAAAGTGGAGGAG | |
| CTGACGAAGAAGGAAGTGGGGGGCGTGGAGCGCGAACAAAGGGGCGTGCAAGATGGACGCCATGAGGGGC | |
| TACGCGGCCAAAGTGGAGGAGCTGACGAAGAAGGAAGTGGGGGGCGTGGAGCGCGAACAAAGGGGCGTCC | |
| AAGATGGACGCCATGA | |

The nucleic acid shown in Table 5 (SEQ ID NO:1) encodes a polypeptide having the amino acid sequence shown in Table 6 (SEQ ID NO:2).

TABLE 6

| | |
|---|---|
| DRVVTATPTLHLQLLAPGTASTTPCAKWSSSXAALKQLKGPVSDQEKLLVYGLYKQATQGDCD | (SEQ ID NO:2) |
| IPGPPASDVRARAKWEAWSANKGASKMDAMRGYAAKVEELTKKE | |

The nucleic acids shown in Table 5A (SEQ ID NO:46 and 48) encode polypeptides having the amino acid sequences shown in Table 6A (SEQ ID NO:47 and 49, respectively)

TABLE 6A

| | |
|---|---|
| >AL121672_GENSCAN_predicted_peptide_5_228_aa | |
| MGDAGATAAALRPAHNLRPAPPTASAAHAQSSRTSAPSAQRRLPAEPSHQPSAPGTASTTPCAKWSSSCA | (SEQ ID NO:47) |
| ALKQLKGPVSDQEKLLVYGLYKQATQGDCDIPGPPASDVRARAKWEAWSANKGASKMDAMRGYAAKVEEL | |
| TKKEVGGVEREQRGVQDGRHEGLRGQSGGADEEGRASKMDAMRGYAAKVEELTKKEVGGVEREQRGVQDG | |
| RHEGLRGQSEEMRKKEAG | |
| >AC025743_GENSCAN_predicted_peptide_7_191_aa | |
| MGDAGATAAALRPAHNLRPAPPTASAAHASPHERARQASRAFRQSPPTSPQLLAPGTASTTPCAKWSSSC | (SEQ ID NO:49) |
| AALKQLKGPVSDQEKLLVYGLYKQATQGDCDIPGPPASDVRARAKWEAWSAKKGASKMDAMRGYAAKVEE | |
| LTKKEVGGVEREQRGVQDGRHEGLRGQSGGADEEGSGGRGARTKGRPRWTP | |

The amino acid residue denoted by “X” can be C or F, i.e., in various embodiments a polypeptide of the invention will include a C at the position denoted by X in the amino acid sequence. In other embodiments, an ENDO1 polypeptide of the invention will include an F at the position denoted by X in the recited amino acid sequence.

An ENDO1 nucleic acid of the invention can include a nucleic acid encoding the polypeptide of SEQ ID NO:2,12, 47, or 49, e.g., the ENDO1 nucleic acid can include the nucleic acid sequence of SEQ ID NO:1, 11, 46, or 48. The invention also includes a mutant or variant nucleic acid any of whose bases may be changed from the corresponding base shown in Table 1 or Table 5. In some embodiments, the ENDO1 nucleic acid encodes a protein that maintains its endozepine-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, but are not limited to: modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

An ENDO1 polypeptide of the invention can include the amino acid sequence of SEQ ID NO: 2,12, 47, or 49. The invention also includes a mutant or variant protein any of whose residues may be changed from the corresponding residue shown in SEQ ID NO:2,12, 47, or 49, while still encoding a protein that maintains its endozepine-like activities and physiological functions, or a functional fragment thereof such as the following active peptide (SEQ ID NO:15).

Metabolism-Regulating Peptide #6 (MRP-6) Sequence:

QATQGDCDIPGPPASDVRAR (SEQ ID NO:15)

A multiple sequence alignment of various embodiments of the ENDO1 polypeptide (Table 6B) displays their relationship to one another.

[image]

Table 6B-1 lists the sequence identifiers and sequence identification numbers (SEQ ID NO) for the sequences displayed in Table 6B.

TABLE 6B-1

| SEQUENCE IDENTIFIER | SEQ ID NO |
|---|---|
| AL121672_GENSCAN_predicted_pep | SEQ ID NO: 150 |
| MRP-6 | SEQ ID NO: 149 |
| AC025743_GENSCAN_predicted_pep | SEQ ID NO: 151 |

The invention further encompasses antibodies and antibody fragments, such as F_{ab} or (F_{ab})₂, that bind immunospecifically to the ENDO1 polypeptide, and derivatives and fragments, thereof.

An ENDO1 sequence is useful for detecting specific types of tissue. For example when a panel of tissue is assayed for expression, ENDO1 is highly expressed in liver and endothelial cells. Also, high expression of ENDO1 is a marker for multiple types of cancer.

An ENDO1 sequence is also useful to modulate global energy metabolism or weight by altering serum glucose or adipose.level

An ENDO1 sequence is also useful in a method to identity the cellular receptors and downstream effectors of the invention by any one of a number of techniques commonly employed in the art.

An ENDO 1 sequence is useful in the treatment of diabetes, metabolic disturbances associated with obesity, the metabolic syndrome X as well as anorexia and wasting disorders associated with chronic diseases and various cancers by modulating metabolism.

ENDO1A

ENDO1A is a variant of ENDO1. An ENDO1A nucleic acid of 687 nucleotides (also referred to as CG57627-04_EXT1) (SEQ ID NO:152) encoding a novel Endozepine-like protein is shown in Table 6C. An open reading frame was identified beginning at nucleotides 183-185 and ending at nucleotides 480-482. The start and stop codons of the open reading frame are highlighted in bold type. Putative untranslated regions (underlined), if any, are found upstream from the initiation codon and downstream from the termination codon.

TABLE 6C

| | | |
|---|---|---|
| ATGGGAGACGCAGGAGCCACGGCGGCCGCGCTTAGGCCTGCTCACAACCTCCGCCCGGCC | 60 | (SEQ ID NO:152) |
| CCGCCCACAGCCTCCGCCGCGCACGCGCAGTCCTCACGAACGAGCGCGCCAAGCGCACAG | 120 | |
| CGCCGCCTTCCGGCAGAGCCCTCCCACCAGCCCTCAGCACCAGGGACCGCCTCCACCACC | 180 | |
| CCATGTGCCAAGTGGAGTTCGAGCTGCGCGGCCCTCAAGCAGCTGAAGGGTCCCGTGAGC | 240 | |
| GATCAGGAGAAGCTGCTGGTCTACGGCTTGTACAAACAGGCCACCCAGGGCGACTGCGAC | 300 | |
| ATCCCCGGCCCTCCGGCCTCAGACGTGAGAGCCAGGGCCAAGTGGGAGGCTTGGAGCGCG | 360 | |
| AACAAAGGGGCGTCCAAGATGGACGCCATGAGGGGCTACGCGGCCAAAGTGGAGGAGCTG | 420 | |
| ACGAAGAAGGAAGTGGGGGGCGTGGAGCGCGAACAAAGGGGCGTGCAAGATGGACGCCAT | 480 | |
| GAGGGGCTACGCGGCCAAAGTGGAGGAGCTGACGAAGAAGGAAGGGCGTCCAAGATGGAC | 540 | |
| GCCATGAGGGGCTACGCGGCCAAAGTGGAGGAGCTGACGAAGAAGGAAGTGGGGGGCGTG | 600 | |
| GAGCGCGAACAAAGGGGCGTCCAAGATGGACGCCATGAGGGGCTACGCGGCCAGAGTGAG | 660 | |
| GAGATGAGGAAGAAGGAGGCTGGCTGA687 | | |

In a search of sequence databases, it was found, for example, that the nucleic acid sequence of ENDO1A has 687 of 687 bases (100%) identical to a gb:GENBANK-ID:AX111785|acc:AX111785.1 mRNA from Homo sapiens (Sequence 46 from Patent WO0125436).

The Endozepine-like ENDO1A disclosed in this invention maps to chromosome 17.

The ENDO1A encoded protein having 99 amino acid residues (SEQ ID NO:153) is presented using the one-letter code in Table 6D. Although PSORT suggests that the Endozepine-like protein may be localized to the cytoplasm with a certainty of 0.4500, the protein of ENDO1A predicted here is similar to the Endozepine family, some members of which are secreted. Therefore it is likely that this novel Endozepine-like protein is an extracellular protein. Alternatively, ENDO1A is likely to be localized to the lysosome lumen with a certainty of 0.2109, to the mitochondrial matrix space with a certainty of 0.1000, or to the microbody (peroxisome) with a certainty of 0.0746.

TABLE 6D

| | | |
|---|---|---|
| MCQVEFELRGPQAAEGSRERSGEAAGLRLVQTGHPGRLRHPRPSGLRRESQGQVGGLERE | 60 | (SEQ ID NO:153) |
| QRGVQDGRHEGLRGQSGGADEEGSGGRGARTKGRARWTP 99 | | |

The full amino acid sequence of ENDO1A was found to have 98 of 99 amino acid residues (98%) identical to, and 98 of 99 amino acid residues (98%) similar to the 99 amino acid residue ptnr:SPTREMBL-ACC:Q9NRE4 protein from Homo sapiens (Human) (ENDOZEPINE-LIKE PROTEIN TYPE 2 MUTANT).

In a further search of public sequence databases, ENDO1A was found to have homology to the amino acid sequences shown in the BLASTP data listed in Table 6E.

TABLE 6E
BLASTP results for ENDO1A

| Gene Index/ | | Length | Identity | Positives | |
|---|---|---|---|---|---|
| Identifier | Protein/Organism | (aa) | (%) | (%) | Expect |
| SPTREMBL- | ENDOZEPINE-LIKE | 99 | 98/99 | 98/99 | 2.1e−49 |
| ACC:Q9NRE4 | PROTEIN TYPE 2 MUTANT | | (98%) | (98%) | |
| | Homo sapiens | | | | |
| SPTREMBL- | ENDOZEPINE-LIKE | 66 | 36/66 | 44/66 | 2.3e−11 |
| ACC:Q9NRE5 | PROTEIN TYPE 1 MUTANT | | (54%) | (66%) | |
| | Homo sapiens | | | | |

A multiple sequence alignment is given in Table 6F in a ClustalW analysis comparing ENDO1A with related protein sequences shown in Table 6E.

[image]

[image]

BLAST analysis was performed on sequences from the Patp database, which is a proprietary database that contains sequences published in patents and patent publications. Patp results include those listed in Table 6G.

TABLE 6G
Patp BLASTP Analysis for ENDO1A

| Sequences | | | | | |
|---|---|---|---|---|---|
| producing | | | | | |
| High- | | | | | |
| scoring | | | | | |
| Segment | | Length | Identity | Positive | |
| Pairs | Protein/Organism | (aa) | (%) | (%) | E Value |
| AAB81804 | Human endozepine- | 191 | 44/47 | 46/47 | 3.9e−20 |
| | like ENDO1 | | (93%) | (97%) | |
| | SEQ ID NO: 49 - | | | | |
| | Homo sapiens | | | | |
| AAB81803 | Human endozepine- | 228 | 31/43 | 35/43 | 1.2e−11 |
| | like ENDO1 | | (72%) | (81%) | |
| | SEQ ID NO: 47 - | | | | |
| | Homo sapiens | | | | |
| AAU32889 | Novel human | 98 | 24/60 | 32/60 | 0.00019 |
| | secreted protein | | (40%) | (53%) | |
| | #3380 - Homo | | | | |
| | sapiens | | | | |
| AAY29514 | Human lung tumour | 595 | 15/29 | 18/29 | 0.0017 |
| | protein SAL-50 1st | | (51%) | (62%) | |
| | predicted amino | | | | |
| | acid sequence - | | | | |
| | Homo sapiens | | | | |
| AAB44463 | Human lung | 595 | 15/29 | 18/29 | 0.0017 |
| | tumour-specific | | (51%) | (62%) | |
| | antigen encoded | | | | |
| | by cDNA #100 - | | | | |
| | Homo sapiens | | | | |

The Endozepine-like ENDO1A disclosed in this invention is expressed in at least the following tissues: Mammalian Tissue, Heart, Colon, Thalamus, Testis, and Lung.

ENDO1B

ENDO1B is a variant of ENDO1. An ENDO1B nucleic acid of 687 nucleotides (also referred to as CG57627-04_EXT2) (SEQ ID NO:156) encoding a novel Endozepine-like protein is shown in Table 6H. An open reading frame was identified beginning at nucleotides 1-3 and ending at nucleotides 685-687. The start and stop codons of the open reading frame are highlighted in bold type. Putative untranslated regions (underlined), if any, are found upstream from the initiation codon and downstream from the termination codon.

TABLE 6H

| | | |
|---|---|---|
| ATGGGAGACGCAGGAGCCACGGCGGCCGCGCTTAGGCCTGCTCACAACCTCCGCCCGGCC | 60 | (SEQ ID NO:156) |
| CCGCCCACAGCCTCCGCCGCGCACGCGCAGTCCTCACGAACGAGCGCGCCAAGCGCACAG | 120 | |
| CGCCGCCTTCCGGCAGAGCCCTCCCACCAGCCCTCAGCACCAGGGACCGCCTCCACCACC | 180 | |
| CCATGTGCCAAGTGGAGTTCGAGCTGCGCGGCCCTCAAGCAGCTGAAGGGTCCCGTGAGC | 240 | |
| GATCAGGAGAAGCTGCTGGTCTACGGCTTGTACAAACAGGCCACCCAGGGCGACTGCGAC | 300 | |
| ATCCCCGGCCCTCCGGCCTCAGACGTGAGAGCCAGGGCCAAGTGGGAGGCTTGGAGCGCG | 360 | |
| AACAAAGGGGCGTCCAAGATGGACGCCATGAGGGGCTACGCGGCCAAAGTGGAGGAGCTG | 420 | |
| ACGAAGAAGGAAGTGGGGGGCGTGGAGCGCGAACAAAGGGGCGTGCAAGATGGACGCCAT | 480 | |
| GAGGGGCTACGCGGCCAAAGTGGAGGAGCTGACGAAGAAGGAAGGGCGTCCAAGATGGAC | 540 | |
| GCCATGAGGGGCTACGCGGCCAAAGTGGAGGAGCTGACGAAGAAGGAAGTGGGGGGCGTG | 600 | |
| GAGCGCGAACAAAGGGGCGTCCAAGATGGACGCCATGAGGGGCTACGCGGCCAGAGTGAG | 660 | |
| GAGATGAGGAAGAAGGAGGCTGGCTGA 687 | | |

The Endozepine-like ENDO1B disclosed in this invention maps to chromosome 17.

In a search of sequence databases, it was found, for example, that the nucleic acid sequence of ENDO1B has 687 of 687 bases (100%) identical to a gb:GENBANK-ID:AXI111785|acc:AX111785.1 mRNA from Homo sapiens (Sequence 46 from Patent WO0125436).

The ENDO1B encoded protein having 228 amino acid residues (SEQ ID NO:157) is presented using the one-letter code in Table 61. Although PSORT suggests that ENDO1B may be localized to the cytoplasm with a certainty of 0.6500, the protein of ENDO1B predicted here is similar to the Endozepine family, some members of which are secreted. Therefore it is likely that this novel Endozepine-like protein is an extracellular protein. Alternatively, ENDO1B is likely to be localized to the mitochondrial matrix space with a certainty of 0.1000 or to the lysosome lumen with a certainty of 0.1000.

TABLE 6I

| | | |
|---|---|---|
| MGDAGATAAALRPAHNLRPAPPTASAAHAQSSRTSAPSAQRRLPAEPSHQPSAPGTASTT | 60 | (SEQ ID NO:157) |
| PCAKWSSSCAALKQLKGPVSDQEKLLVYGLYKQATQGDCDIPGPPASDVRARAKWEAWSA | 120 | |
| NKGASKMDAMRGYAAKVEELTKKEVGGVEREQRGVQDGRHEGLRGQSGGADEEGRASKMD | 180 | |
| AMRGYAAKVEELTKKEVGGVEREQRGVQDGRHEGLRGQSEEMRKKEAG 228 | | |

The full amino acid sequence of ENDO1B was found to have 62 of 81 amino acid residues (76%) identical to, and 70 of 81 amino acid residues (86%) similar to, the 87 amino acid residue ptnr:SWISSNEW-ACC:009035 protein from Mus musculus (Mouse) (DIAZEPAM BINDING INHIBITOR-LIKE 5 (ENDOZEPINE-LIKE PEPTIDE) (ELP)).

In a further search of public sequence databases, ENDO1B was found to have homology to the amino acid sequences shown in the BLASTP data listed in Table 6J.

TABLE 6J
BLASTP results for ENDO1B

| | | | | Pos- | |
|---|---|---|---|---|---|
| Gene Index/ | | Length | Identity | itives | |
| Identifier | Protein/Organism | (aa) | (%) | (%) | Expect |
| SWISSPROT- | Diazepam binding | 87 | 61/83 | 71/83 | 1.1e−29 |
| ACC: | inhibitor-like 5 | | (73%) | (85%) | |
| Q9MZG3 | (Endozepine-like | | | | |
| | peptide) (ELP) - | | | | |
| | Bos taurus | | | | |
| SWISSPROT- | Diazepam binding | 87 | 62/81 | 70/81 | 7.5e−29 |
| ACC: 009035 | inhibitor-like 5 | | (76%) | (86%) | |
| | (Endozepine-like | | | | |
| | peptide) (ELP) - | | | | |
| | Mus musculus | | | | |
| SWISSPROT- | Diazepam binding | 87 | 60/81 | 69/81 | 1.2e−28 |
| ACC: P56702 | inhibitor-like 5 | | (74%) | (85%) | |
| | (Endozepine-like | | | | |
| | peptide) (ELP) - | | | | |
| | Rattus norvegicus | | | | |
| SPTREMBL- | ENDOZEPINE- | 80 | 57/76 | 69/76 | 1.6e−28 |
| ACC: | LIKE PROTEIN - | | (75%) | (90%) | |
| Q9MZG0 | Callithrix jacchus | | | | |
| SPTREMBL- | ENDOZEPINE- | 59 | 50/56 | 52/56 | 5.1e−23 |
| ACC: | LIKE PROTEIN - | | (89%) | (92%) | |
| Q9MZG1 | Macaca | | | | |
| | fascicularis | | | | |

A multiple sequence alignment is given in Table 6K in a ClustalW analysis comparing ENDO1A with related protein sequences shown in Table 6J.

[image]

BLAST analysis was performed on sequences from the Patp database, which is a proprietary database that contains sequences published in patents and patent publications. Patp results include those listed in Table 6L.

TABLE 6L
Patp BLASTP Analysis for ENDO1B

| Sequences | | | | | |
|---|---|---|---|---|---|
| producing High- | | | | | |
| scoring Segment | | Length | Identity | Positive | |
| Pairs | Protein/Organism | (aa) | (%) | (%) | E Value |
| AAB81803 | Human endozepine-like | 228 | 228/228 | 228/228 | 7.5e−120 |
| | ENDO1 SEQ ID NO: 47 - | | (100%) | (100%) | |
| | Homo sapiens | | | | |
| AAB81804 | Human endozepine-like | 191 | 157/185 | 159/185 | 3.3e−78 |
| | ENDO1 SEQ ID NO: 49 - | | (84%) | (85%) | |
| | Homo sapiens | | | | |
| AAB81802 | Human endozepine-like | 107 | 96/106 | 98/106 | 2.3e−45 |
| | ENDO1 SEQ ID NO: 2 - | | (90%) | (92%) | |
| | Homo sapiens | | | | |
| AAB81801 | Human endozepine-like | 89 | 89/89 | 89/89 | 2.1e−44 |
| | ENDO1 SEQ ID NO: 12 - | | (100%) | (100%) | |
| | Homo sapiens | | | | |
| AAB11967 | Human diazepam binding | 104 | 41/93 | 53/93 | 2.3e−15 |
| | inhibitor (DBI) - Homo | | (44%) | (56%) | |
| | sapiens | | | | |

The presence of identifiable domains in the protein disclosed herein was determined by searches versus domain databases such as Pfam, PROSITE, ProDom, Blocks or Prints and then identified by the Interpro domain accession number. Significant domains are summarized in Table 6M.

TABLE 6M

| Model | Domain | seq-f | seq-t | hmm-f | hmm-t | score | E-value |
|---|---|---|---|---|---|---|---|
| -------- | ------- | ----- | ----- | ----- | ----- | ----- | ------- |
| ACBP | 1/1 | 69 | 145 . . | 1 | 89 [] | 118.1 | 1.7e−31 |

Alignments of top-scoring domains:
ACBP: domain 1 of 1, from 69 to 145: score 118.1, E = 1.7e−31

| | | | | |
|---|---|---|---|---|
| | | *->lqedFeaAaekvkkLkknGpvkPSneekLkLYsLYKQATvGDvnter | | (SEQ ID NO:163) |
| | | ++++\|+\|\| + +\|++\|\|\| +\|+\|\|\|\|\|\|+\|\|++++ | | |
| ENDO1B | 69 | --------CAALKQLKGP----VSDQEKLLVYGLYKQATQGDCDIPG | 103 | (SEQ ID NO:164) |
| | | PGmfDlkgrAKWDAWnelkGmSkeeAmkaYIakVeeLiakya | | |
| | | \|+ \|++ \|\|\|\|+\|\|+++\|\| \|\|++\|\|+ \|+\|\|\|\|\| +\|+ | | |
| ENDO1B | 104 | PPASDVRARAKWEAWSANKGASKMDAMRGYAAKVEELTKKEV 145 | | |

Acyl-CoA-binding protein (ACBP) is a small (10 Kd) protein that binds medium- and long-chain acyl-CoA esters with high affinity, and may act as an intra-cellular carrier of acyl-CoA esters. ACBP has a number of important physiological and biochemical functions: it is known as a diazepam binding inhibitor, as a putative neurotransmitter, as a regulator of insulin release from pancreatic cells, and as a mediator in corticotropin-dependent adrenal steroidogenesis. It is possible that the protein acts as a neuropeptide that takes part in the modulation of gamma-aminobutyric acid-ergic transmission. The structure of ACBP has been deduced by NMR spectroscopy and has been shown to be a mainly-alpha protein, consisting of short alpha-helices and 3 connecting beta-strands.

This indicates that the sequence of the invention has properties similar to those of other proteins known to contain this/these domain(s) and similar to the properties of these domains.

The Endozepine-like gene disclosed in this invention is expressed in at least the following tissues: Mammalian Tissue, Heart, Colon, Thalamus, Testis, and Lung.

The nucleic acids and proteins of the invention have applications in the diagnosis and/or treatment of various diseases and disorders. For example, the compositions of the present invention will have efficacy for the treatment of patients suffering from: anxiety disorders and CNS disorders where GABA neuro-transmitters are involved as well as other diseases, disorders and conditions.

These materials are further useful in the generation of antibodies that bind immunospecifically to the novel substances of the invention for use in diagnostic and/or therapeutic methods.

ENDO2

An ENDO2 nucleic acid of the invention includes the nucleic acid sequence shown in Table 7 (SEQ ID NO:3). An ORF, as well as putative untranslated regions upstream from the initiation codon and downstream from the stop codon of the ORF, is present in the nucleotide sequence disclosed in Table 7. Untranslated nucleotides are shown by underlining. The start and stop codons of the ORF are shown in bold letters.

TABLE 7

| | |
|---|---|
| GTATAAGACATACAGAAGGAATGCCTGGAGAGCAGCAACAGCCCAGCTGCGGCCACCATGTCC | (SEQ ID NO:3) |
| CTGCAGGCTGATTTTGACATGGTCACAGAAGATGTGAGGAAGCTGAAAACAAGACCAGATGAT | |
| GAAGAACTGAAAGAACTTTATGGGCTTTACAAACAAGCTGTAATTGGAAACATTAATATTGAG | |
| TGTTCAGAAATGCTAGAATTAAAAGGCAAGGCCAAATGGGAAGCACAGAACCCCCAAAAAGGA | |
| TTGTCAGAGGAAGATATGATGCGTGCCTTTATTTCTAAAGCCGAAGAGCTGATAGAAAAATAT | |
| GGAATTTAGAATAAAGCATATGATAAATTTTCCTTT | |

The nucleic acid sequence of Table 7 has 218 of 294 bases (74%) identical and positive to a 470 nucleotide Rana ridibunda diazepam-binding inhibitor (DBI) mRNA (GENBANK-ID: RRU09205|acc:U09205). A BLASTN identify search comparing regions of the sequence disclosed in Table 7 to the Rana ridbunda DPI mRNA is shown in Table 8. Regions of the disclosed sequence is are presented as “Query” sequences, and the Rana ridbunda DPI mRNA (SEQ ID NO:11) sequences are presented as the “Subject sequences”.

TABLE 8

| | | | | |
|---|---|---|---|---|
| Query: | 46 | GCTGCGGCCACCATGTCCCTGCAGGCTGATTTTGACATGGTCA-CAGAAGATGTGAGGAA | 104 | (SEQ ID NO:50) |
| | | \|\|\|\| \| \|\|\|\|\|\|\|\| \| \|\|\|\|\| \|\|\|\|\|\|\|\|\| \| \|\| \|\|\| \|\|\|\|\| \| \|\| | | |
| Sbjct: | 1 | GCTGAATCAACCATGTCACCCCAGGCAGATTTTGACAAAG-CAGCAGGGGATGTAAAGAA | 59 | (SEQ ID NO:51) |
| Query: | 105 | GCTGAAAACAAGACCAGATGATGAAGAACTGAAAGAACTTTATGGGCTTTACAAACAAGC | 164 | |
| | | \|\|\|\|\|\|\|\|\| \|\|\|\| \|\|\| \|\| \|\|\|\|\|\|\|\| \|\|\|\|\| \|\| \|\| \|\| \|\|\|\|\| \|\| \| | | |
| Sbjct: | 60 | ATTGAAAACAAAACCAACTGACGATGAACTGAAGGAACTGTACGGACTCTACAAGCAGTC | 119 | |
| Query: | 165 | TGTAATTGGAAACATTAATATTGAGTGTTCAGAAATGCTAGAATTAAAAGGCAAGGCCAA | 224 | |
| | | \|\|\|\| \|\|\|\| \|\|\|\|\| \|\|\|\|\|\| \| \| \|\|\|\|\|\|\|\| \| \|\| \|\|\|\|\|\|\|\|\|\|\| | | |
| Sbjct: | 120 | CACTGTTGGGGACATAAATATAGAGTGTCCTGGCATGCTAGATCTGAAGGGCAAGGCCAA | 179 | |
| Query: | 225 | ATGGGAAGCACAGAACCCCCAAAAAGGATTGTCAGAGGAAGATATGATGCGTGCCTTTAT | 284 | |
| | | \|\|\|\|\| \|\|\| \|\|\|\|\| \| \|\|\|\|\| \|\|\|\|\| \|\|\|\|\|\|\|\| \|\|\|\| \| \|\| \| \| \| | | |
| Sbjct: | 180 | GTGGGACGCATGGAACCTAAAGAAAGGCTTGTCTAAGGAAGATGCGATGAGCGCTTATGT | 239 | |
| Query: | 285 | TTCTAAAGCCGAAGAGCTGATAGAAAAATATGGAATTTAGAATAAAG-CATATGAT 339 | | |
| | | \|\|\|\|\|\|\|\|\|\| \| \|\|\|\|\|\|\|\|\|\|\|\|\|\|\|\|\|\|\|\| \| \|\| \| \|\| \| \| \|\|\|\|\| | | |
| Sbjct: | 240 | TTCTAAAGCCCATGAGCTGATAGAAAAATATGGCCTGTA-AC-AAGGTCGCATGAT 293 | | |

The ORF encodes a polypeptide of 85 amino acids (SEQ ID NO:4). The amino acid sequence of this polypeptide is shown in Table 9 (SEQ ID NO:4).

TABLE 9

| | |
|---|---|
| MSLQADFDMVTEDVRKLKTRPDDEELKELYGLYKQAVIGNINIECSEMLELKGKAKWEAQNPQ | (SEQ ID NO:4) |
| KGLSEEDMMRAFISKAEELIEKYGI | |

The polypeptide sequence disclosed in Table 9 is related to a previously described duck diazepam binding inhibitor polypeptide. This relationship is shown in Table 10. The amino acid sequence shown in Table 9 (SEQ ID NO:4) has 60 of 85 amino acid residues (70%) identical to, and 72 of 85 residues (84%) positive with, the 103 amino acid residue acyl-coA-binding protein (ACBP) (diazepam binding inhibitor)(DBI) (endozepine) (EP) from Anas platyrhynchos (domestic duck) (ptnr: SWISSPROT-ACC:P45882). Regions of the polypeptide sequnce shown in Table 9 are presented as the “Query” sequence. Regions of the duck polypeptide sequence are shown as the “Sbct” sequences.

TABLE 10

| | | | | |
|---|---|---|---|---|
| Query: | 67 | QADFDMVTEDVRKLKTRPDDEELKELYGLYKQAVIGNINIECSEMLELKGKAKWEAQNPQ | 246 | (SEQ ID NO:52) |
| | | \|\|\|\|\| \|+\|+\|\|\|\|\|\| \|\|\|\|\|\|\|\|\| \|\|\|\| +\|+\|\|\|\|\| \|\|+\|\|\|\|\|\|\|\|\| \| + | | |
| Sbjct: | 19 | QADFDEAAEEVKKLKTRPTDEELKELYGFYKQATVGDINIECPGMLDLKGKAKWEAWNLK | 78 | (SEQ ID NO:53) |
| Query: | 247 | KGLSEEDMMRAFISKAEELIEKYGI 321 | | |
| | | \|\|+\|+\|\| \| \|+\|\|\|\|+ ++\|\|\|\|\| | | |
| Sbjct: | 79 | KGISKEDAMNAYISKAKTMVEKYGI 103 | | |

A multiple sequence alignment between the amino acids of SEQ ID NO:4 and various acyl coA binding polypeptides is illustrated in Table 11. Shown is an alignment between the amino acid sequence of Table 7 (“ACBP_Novel”), porcine acyl-coA binding protein (SWISSPROT locus ACBP_PIG, accession No. P12026) (“ACBP_PIG”), bovine acyl-coA binding protein (SWISSPROT locus ACBP_BOVIN, accession no. P07107)(“ACBP_BOVIN”), and human acyl-coA binding protein (SWISSPROT locus ACBP_HUMAN, accession no. P07108)(“ACBP_HUMAN”). Regions of perfect homology are shown in black. Regions with conservative amino acid substitutions are shown in gray. Non-conservative amino acid substitutions are presented without shading.

[image]

Table 11-1 lists the sequence identifiers and sequence identification numbers (SEQ ID NO) for the sequences displayed in Table 11.

TABLE 11-1

| SEQUENCE IDENTIFIER | SEQ ID NO |
|---|---|
| ACBP_PIG | SEQ ID NO: 54 |
| ACBP_BOVIN | SEQ ID NO: 55 |
| ACBP_HUMAN | SEQ ID NO: 56 |
| ACBP_Novel | SEQ ID NO: 57 |

An ENDO2 nucleic acid of the invention encoding a endozepine-like protein includes the nucleic acid encoding a polypeptide that includes the amino acid sequence of SEQ ID NO:4, e.g., a nucleic acid whose sequence is provided in SEQ ID NO:3. The invention also includes a fragment of SEQ ID NO:3, or a mutant or variant nucleic acid any of whose bases may be changed from the corresponding base shown in SEQ ID NO:3. In some embodiments, the mutant or variant nucleic acid encodes a protein that maintains its endozepine-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, but are not limited-to, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

An ENDO2 polypeptide according to the invention includes a polypeptide comprising the an amino acid sequence shown in Table 9 (SEQ ID NO:4). The invention also includes a mutant or variant protein any of whose residues may be changed from the corresponding residue shown in Table 8, while still encoding a protein that maintains its endozepine-like activities and physiological functions, or a functional fragment thereof such as the following active peptide (SEQ ID NO: 16)

Metabolism-Regulating Peptide #5 (MRP-5) Sequence:

QAVIGNINIECSEMLELKGK (SEQ ID NO: 16).

The invention further encompasses antibodies and antibody fragments, such as F_{ab} or (F_{ab})₂, that bind immunospecifically to the ENDO2 polypeptide, and derivatives and fragments, thereof.

An ENDO2 sequence is useful for detecting specific types of tissue. For example when a panel of tissue is assayed for expression, ENDO2 is highly expressed in brain and pancreas cells. Also, high expression of ENDO2 is a marker for colon and lung cancer.

An ENDO2 sequence is also useful to modulate global energy metabolism or weight by altering serum glucose or adipose level.

An ENDO2 sequence is also useful in a method to identity the cellular receptors and downstream effectors of the invention by any one of a number of techniques commonly employed in the art.

An ENDO2 sequence is useful in the treatment of diabetes, metabolic disturbances associated with obesity, the metabolic syndrome X as well as anorexia and wasting disorders associated with chronic diseases and various cancers by modulating metabolism.

ENDO2A

ENDO2A is a variant of ENDO2. An ENDO2A nucleic acid of 330 nucleotides (also referred to as CG53366-O₂) (SEQ ID NO: 165) encoding a novel Diazepam binding inhibitor-like protein is shown in Table 11A. An open reading frame was identified beginning at nucleotides 47-49 and ending at nucleotides 311-313. The start (ATG) and stop (TAG) codons of the open reading frame are highlighted in bold type. Putative untranslated regions (underlined), if any, are found upstream from the initiation codon and downstream from the termination codon.

TABLE 11A

| | | |
|---|---|---|
| ACAGAAGGAATGCCTGGAGAGCAGCAACAGCCCAGCTGCGGCCACCATGTCCCTGCAGGC | 60 | (SEQ ID NO:165) |
| TGATTTTGACATGGTCACAGAAGATGTGAGGAAGCTGAAAACAAGACCAGATGATGAAGA | 120 | |
| ACTGAAAGAACTTTATGGGCTTTACAAACAAGCTGTAATTGGAAACATTAATATTGAGTG | 180 | |
| TTCAGAAATGCTAGAATTAAAAGGCAAGGCCAAATGGGAAGCACAGAACCCCCAAAAAGG | 240 | |
| ATTGTCAGAGGAAGATATGATGCGTGCCTTTATTTCTAAAGCCGAAGAGCTGATAGAAAA | 300 | |
| ATATGGAATTTAGAATAAAGCATATGATAA 330 | | |

The Diazepam binding inhibitor-like ENDO2A disclosed in this invention maps to chromosome 15q21.

In a search of sequence databases, it was found, for example, that ENDO2A has 218 of 294 bases (74%) identical to a gb:GENBANK-ID:RRU09205|acc:U09205.1 mRNA from Rana ridibunda (Rana ridibunda diazepam-binding inhibitor (DBI) mRNA, complete cds).

The encoded protein having 88 amino acid residues (SEQ ID NO:166) is presented using the one-letter code in Table 11B. Although the PSORT, SignalP and hydropathy profile results predict that this sequence has no signal peptide and is likely to be localized in the cytoplasm with a certainty of 0.45000, proteins of this family are generally secreted and hence we predict that ENDO2A is likely to be an extracellular protein. Alternatively, ENDO2A is likely to be localized to the mitochondrial matrix space with a certainty of 0.3600 or to the lysosome lumen with a certainty of 0.1000.

TABLE 11B

| | | |
|---|---|---|
| MSLQADFDMVTEDVRKLKTRPDDEELKELYGLYKQAVIGNINIECSEMLELKGKAKWEAQ | 60 | (SEQ ID NO:166) |
| NPQKGLSEEDMMRAFISKAEELIEKYGI 88 | | |

The full amino acid sequence of ENDO2A was found to have 68 of 88 amino acid residues (77%) identical to, and 76 of 88 amino acid residues (86%) similar to, the 88 amino acid residue ptnr:TREMBLNEW-ACC:BAB32079 protein from Mus musculus (Mouse) (ADULT MALE EPIDIDYMIS cDNA, RIKEN FULL-LENGTH ENRICHED LIBRARY, CLONE:9230116B 18, FULL INSERT SEQUENCE).

In a further search of public sequence databases, ENDO2A was found to have homology to the amino acid sequences shown in the BLASTP data listed in Table 11C.

TABLE 11C
BLASTP results for ENDO2A

| Gene Index/ | | Length | Identity | Positives | |
|---|---|---|---|---|---|
| Identifier | Protein/Organism | (aa) | (%) | (%) | Expect |
| SPTREMBL- | 9230116B18RIK PROTEIN - | 88 | 68/88 | 76/88 | 1.9e−30 |
| ACC: Q9D258 | Mus musculus | | (77%) | (86%) | |
| pir-id: A57711 | diazepam-binding | 88 | 61/88 | 74/88 | 7.5e−29 |
| | inhibitor - laughing frog | | (69%) | (84%) | |
| SWISSPROT- | Acyl-CoA-binding | 103 | 60/85 | 72/85 | 1.2e−28 |
| ACC: P45882 | protein (ACBP) | | (70%) | (84%) | |
| | (Diazepam binding | | | | |
| | inhibitor) (DBI) | | | | |
| | (Endozepine) (EP) - | | | | |
| | Anas platyrhynchos | | | | |
| SWISSPROT- | Acyl-CoA-binding | 87 | 60/87 | 73/87 | 2.5e−28 |
| ACC: P45883 | protein homolog (ACBP) | | (68%) | (83%) | |
| | (Diazepam binding | | | | |
| | inhibitor homolog) - (DBI) | | | | |
| | Rana ridibunda | | | | |
| SWISSPROT- | Acyl-CoA-binding | 86 | 48/85 | 63/85 | 4.1e−21 |
| ACC: P07108 | protein (ACBP) | | (56%) | (74%) | |
| | (Diazepam binding | | | | |
| | inhibitor) (DBI) | | | | |
| | (Endozepina) (EP) - Homo sapiens | | | | |

A multiple sequence alignment is given in Table 11D in a ClustalW analysis comparing ENDO1A with related protein sequences shown in Table 11C.

[image]

BLAST analysis was performed on sequences from the Patp database, which is a proprietary database that contains sequences published in patents and patent publications. Patp results include those listed in Table 11E.

TABLE 11E
Patp BLASTP Analysis for ENDO2A

| Sequences | | | | | |
|---|---|---|---|---|---|
| producing High- | | | | | |
| scoring Segment | | Length | Identity | Positive | |
| Pairs | Protien/Organism | (aa) | (%) | (%) | E Value |
| AAB81806 | Human endozepine-like | 88 | 88/88 | 88/88 | 4.5e−42 |
| | ENDO2 SEQ ID NO: 4 - | | (100%) | (100%) | |
| | Homo sapiens | | | | |
| AAG78314 | Human DBI/ACBP-like | 88 | 88/88 | 88/88 | 4.5e−42 |
| | protein - Homo sapiens | | (100%) | (100%) | |
| AAB81811 | Human endozepine-like | 96 | 64/86 | 72/86 | 2.4e−29 |
| | ENDO4 SEQ ID NO: 8 - | | (74%) | (83%) | |
| | Homo sapiens | | | | |
| AAB53603 | Human colon cancer | 111 | 49/87 | 65/87 | 1.0e−21 |
| | antigen protein | | (56%) | (74%) | |
| | sequence SEQ ID NO: 1143 - | | | | |
| | Homo sapiens | | | | |
| AAP60955 | Sequence of human | 86 | 48/85 | 63/85 | 3.4e−21 |
| | endogenous | | (56%) | (74%) | |
| | benzodiazepineoid | | | | |
| | (EBZD) polypeptide - | | | | |
| | Homo sapiens | | | | |

The presence of identifiable domains in the protein disclosed herein was determined by searches versus domain databases such as Pfam, PROSITE, ProDom, Blocks or Prints and then identified by the Interpro domain accession number. Significant domains are summarized in Table 11F.

TABLE 11F

| Model | Domain | seq-f | seq-t | hmm-f | hmm-t | score | E-value |
|---|---|---|---|---|---|---|---|
| -------- | ------- | ----- | ----- | ----- | ----- | ----- | ------- |
| ACBP | 1/1 | 3 | 87 .. | 1 | 89 [] | 116.0 | 7.2e−31 |

Alignments of top-scoring domains:
ACBP: domain 1 of 1, from 3 to 87: score 116.0, E=7.2e-31

| | | | | |
|---|---|---|---|---|
| | | lqedFeaAaekvkkLkknGpvkPSneekLkLYsLYKQATvGDvnter | | (SEQ ID NO:172) |
| | | \|\|+\|\|++ \| \|+\|\|\|++ \|++\|\|+ +\|\|+\|\|\|\|\| +\| +\|+\|+ | | |
| ENDO2A | 3 | LQADFDMVTEDVRKLKTR----PDDEELKELYGLYKQAVIGNINIEC | 45 | (SEQ ID NO:173) |
| | | PGmfDlkgrAKWDAWnelkGmSkeeAmkaYIakVeeLiakya | | |
| | | \|+ \|\|\|+\|\|\|+\| \| +\|\|+\|+\|+ \|+\| \| \| \|\|\|\|+\|\| | | |
| ENDO2A | 46 | SEMLEKLGKAKWEAQNPQKGLSEEDMMRAFISKAEELIEKYG 87 | | |

Acyl-CoA-binding protein (ACBP) is a small (10 Kd) protein that binds medium- and long-chain acyl-CoA esters with high affinity, and may act as an intra-cellular carrier of acyl-CoA esters. ACBP has a number of important physiological and biochemical functions: it is known as a diazepam binding inhibitor, as a putative neurotransmitter, as a regulator of insulin release from pancreatic cells, and as a mediator in corticotropin-dependent adrenal steroidogenesis. It is possible that the protein acts as a neuropeptide that takes part in the modulation of gamma-aminobutyric acid-ergic transmission. The structure of ACBP has been deduced by NMR spectroscopy and has been shown to be a mainly-alpha protein, consisting of short alpha-helices and 3 connecting beta-strands (Marquardt H et al., 1986, J. Biol. Chem. 261: 9727-9731; Poulsen F. M., Andersen K. V., 1992, J. Mol. Biol. 226: 1131-1141). ACBP is a highly conserved protein of about 90 residues that has been so far found in vertebrates, insects, plants and yeast. Other proteins belonging to the ACBP family include mouse endozepine-like peptide (ELP) (gene DBIL5); mammalian MA-DBI, a transmembrane protein of unknown function which has been found in mammals; and human DRS-1, a protein of unknown function that contains a N-terminal ACBP-like domain and a C-terminal enoyl-CoA isomerase/hydratase domain (Ivell R. et al., 1996, Mol. Cell. Endocrinol. 122: 69-80; Suk K et al., 1999, Biochim. Biophys. Acta 1454:126-131).

This indicates that the sequence of the invention has properties similar to those of other proteins known to contain this/these domain(s) and similar to the properties of these domains.

The Diazepam binding inhibitor-like gene disclosed in this invention is expressed in at least the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus.

The nucleic acids and proteins of the invention have applications in the diagnosis and/or treatment of various diseases and disorders. For example, the compositions of the present invention will have efficacy for the treatment of patients suffering from: cancer, trauma, immunological disease, respiratory disease, gastro-intestinal diseases, reproductive health, neurological and neurodegenerative diseases, bone marrow transplantation, metabolic and endocrine diseases, allergy and inflammation, nephrological disorders, hematopoietic disorders or urinary system disorders as well as other diseases, disorders and conditions.

These materials are further useful in the generation of antibodies that bind immunospecifically to the novel substances of the invention for use in diagnostic and/or therapeutic methods.

ENDO2B

ENDO2B is a variant of ENDO2. An ENDO2B nucleic acid of the invention (also referred to as CG53366-03) (SEQ ID NO:174) includes the nucleic acid sequence shown in Table 11G. The ORF begins with an ATG initiation codon at nucleotides 398-400 and ends with a TAG codon at nucleotides 662-664. Putative untranslated regions upstream from the initiation codon and downstream from the stop codon are shown in Table 11G by underling, and the start and stop codons are shown in bold letters.

TABLE 11G

| | | |
|---|---|---|
| TAATGGGCGCACAACATATAAAGATATAATTTGTGACAATCACAACATAAAGTATGGGCA | 60 | (SEQ ID NO:174) |
| GCGCTGTATAGAGCTATAGAGCAGAGATTTTTGTATGCTATCAAAGCTAAATTTGGATCA | 120 | |
| ATTTAAACTAGGTTGTTATAAATTTATGAAGTTGATTACCTCTGTGGTAACCACTTAAAA | 180 | |
| TTTTTTTAATTTTAATTTTTATTTATTTTTTGAGACGGAGTCTCACTCTGTCTCTAAAAA | 240 | |
| AAGGTCAAGAAAATTAGAAGGGTATTAAATGATACACTACAAAAAAAAATCAATGGAATA | 300 | |
| CAAAAGAAGGCAGTAGTGGAGGAAATGAGGAACAAAAATGGTATAAGACATACAGAAGGA | 360 | |
| ATGCCTGGAGAGCAGCAACAGCCCAGCTGCGGCCACCATGTCCCTGCAGGCTGATTTTGA | 420 | |
| CATGGTCACAGAAGATGTGAGGAAGCTGAAAACAAGACCAGATGATGGAGAACTGAAAGA | 480 | |
| ACTCTATGGGCTTTACAAACAAGCTGTAATTGGAAACATTAATATTGAGTGTTCAGAAAT | 540 | |
| GCTAGATTTAAAAGGCAAAGCCAAATGGGAAGCATGGAACCCCCAAAAAGGATTGTCGAC | 600 | |
| GGAAGATATGATGCGTGCCTTTATTTCTAAAGCCGAAGAGCTGATAGAAAAATATGGAAT | 660 | |
| TTAGAATAAAGCATATGATAAATTTTCCTTTTTGAAGCCTTCATAATGGTATCATGACCA | 720 | |
| AACATTTAGAGTTAACGCTGTTAACTCTAGGTATCATGTATATTTTTGCTATTATTATGA | 780 | |
| ATTATACTTAATTAGTAGTATGCTAAAACTGCATAGTTAACTAAATTGTACTTGCTTAAA | 840 | |
| CCAGGTGTCTTTAAAAGTTCTTTTAGAAAAGTATTTTTTTTATTTTTATAGATTTAGGGG | 900 | |
| GTACAAGTGCAGTTTTGTTGCATGAACGTATCATGTAGTGGTGAAGTCTGGGCTTTCAGT | 960 | |
| GTCCCCATCACCCAGATAGTCTACAATTGTGCCCAAAAGGTACAATTGTACATTCCTTAC | 1020 | |
| ACCTTCTGTGACCATGTCAAAATCAGCCT 1049 | | |

The disclosed ENDO2B nucleic acid sequence (SEQ ID NO:174) has 309 of 455 bases (67%) identical to Rana ridibunda diazepam-binding inhibitor (DBI) (GENBANK-ID: RRU09205|acc:U09205.1).

The ORF identified in Table 11G encodes a polypeptide sequence of 88 residues (SEQ ID NO:175), which is presented in Table 11H. Although the PSORT, SignalP and hydropathy profile results predict that this sequence has no signal peptide and is likely to be localized in the cytoplasm with a certainty of 0.45000, proteins of this family are generally secreted and hence we predict that ENDO2B is likely to be an extracellular protein. Alternatively, ENDO2B is likely to be localized to the mitochondrial matrix space with a certainty of 0.3600 or to the lysosome lumen with a certainty of 0.1000.

TABLE 11H

| | | |
|---|---|---|
| MSLQADFDMVTEDVRKLKTRPDDGELKELYGLYKQAVIGNINIECSEMLDLKGKAKWEAW | 60 | (SEQ ID NO:175) |
| NPQKGLSTEDMMRAFISKAEELIEKYGI 88 | | |

The amino acid sequence of ENDO2B (SEQ ID NO:175) has 63 of 88 residues (71%) identical to, and 73 of 88 residues (82%) similar to, laughing frog diazepam-binding inhibitor (pir-id: A57711).

In a further search of public sequence databases, ENDO2B was found to have homology to the amino acid sequences shown in the BLASTP data listed in Table 11I.

TABLE 11I
BLASTP results for ENDO2B

| Gene Index/ | | Length | Identity | Positives | |
|---|---|---|---|---|---|
| Identifier | Protien/Organism | (aa) | (%) | (%) | Expect |
| SPTREMBL- | 9230116B18RIK PROTEIN - | 88 | 69/88 | 75/88 | 2.7e−31 |
| ACC: Q9D258 | Mus musculus | | (78%) | (85%) | |
| pir-id: A57711 | diazepam-binding | 88 | 63/88 | 73/88 | 4.0e−30 |
| | inhibitor - laughing | | (71%) | (82%) | |
| | frog | | | | |
| SWISSPROT- | Acyl-CoA-binding | 87 | 62/87 | 72/87 | 1.4e−29 |
| ACC: P45883 | protein homolog (ACBP) | | (71%) | (82%) | |
| | (Diazepam binding | | | | |
| | inhibitor homolog) | | | | |
| | (DBI) - Rana ridibunda | | | | |
| SWISSPROT- | Acyl-CoA-binding | 103 | 61/85 | 71/85 | 1.7e−29 |
| ACC: P45882 | protein (ACBP) | | (71%) | (83%) | |
| | (Diazepam binding inhibitor) | | | | |
| | (DBI) (Endozepine) | | | | |
| | (EP) - Anas platyrhynchos | | | | |
| SWISSPROT- | Acyl-CoA-binding | 86 | 49/85 | 62/85 | 5.8e−22 |
| ACC: P07108 | protein (ACBP) | | (57%) | (72%) | |
| | (Diazepam binding | | | | |
| | inhibitor) (DBI) | | | | |
| | (Endozepine) (EP) - Homo sapiens | | | | |

A multiple sequence alignment is given in Table 11J in a ClustalW analysis comparing ENDO1B with related protein sequences shown in Table 11I.

[image]

[image]

BLAST analysis was performed on sequences from the Patp database, which is a proprietary database that contains sequences published in patents and patent publications. Patp results include those listed in Table 11K.

TABLE 11K
Patp BLASTP Analysis for ENDO2B

| Sequences | | | | | |
|---|---|---|---|---|---|
| producing High- | | | | | |
| scoring Segment | | | | | |
| Pairs | Protein/Organism | Length (aa) | Identity (%) | Positive (%) | E Value |
| AAB81806 | Human endozepine-like | 88 | 84/88 | 85/88 | 1.2e−39 |
| | ENDO2 SEQ ID NO: 4 - | | (95%) | (96%) | |
| | Homo sapiens | | | | |
| AAE09445 | Human sbg35O69DBIa | 88 | 84/88 | 85/88 | 1.2e−39 |
| | protein - Homo sapiens | | (95%) | (96%) | |
| AAG78314 | Human DBI/ACBP-like | 88 | 84/88 | 85/88 | 1.2e−39 |
| | protein - Homo sapiens | | (95%) | (96%) | |
| AAB81811 | Human endozepine-like | 96 | 68/86 | 75/86 | 1.2e−32 |
| | ENDO4 SEQ ID NO: 8 - | | (79%) | (87%) | |
| | Homo sapiens | | | | |
| AA553603 | Human colon cancer | 111 | 50/87 | 64/87 | 1.4e−22 |
| | antigen protein | | (57%) | (73%) | |
| | sequence SEQ ID | | | | |
| | NO:1143 - Homo sapiens | | | | |

The presence of identifiable domains in the protein disclosed herein was determined by searches versus domain databases such as Pfam, PROSITE, ProDom, Blocks or Prints and then identified by the Interpro domain accession number. Significant domains are summarized in Table 11.

TABLE 11L

| Model | Domain | seq-f | seq-t | hmm-f | hmm-t | score | E-value |
|---|---|---|---|---|---|---|---|
| -------- | ------- | ----- | ----- | ----- | ----- | ----- | ------- |
| ACBP | 1/1 | 3 | 87 | 1 | 89 | 129.0 | 8.8e−35 |

Alignments of top-scoring domains:
ACBP: domain 1 of 1, from 3 to 87: score 129.0, E = 8.8e−35

| | | | | |
|---|---|---|---|---|
| | | lqedFeaAaekvkkLkknGpvkPSneekLkLYsLYKQATvGDvnter | | (SEQ ID NO:172) |
| | | \|\|+\|\|++ \| \|+\|\|\|++ \|++ \|+ +\|\|+\|\|\|\|\| +\| +\|+\|+ | | |
| ENDO2A | 3 | LQADFDMVTEDVRKLKTR----PDDGELKELYGLYKQAVIGNINIEC | 45 | (SEQ ID NO:173) |
| | | PGmfDlkgrAKWDAWnelkGmSkeeAmkaYIakVeeLiakya | | |
| | | \|+\|\|\|\|+\|\|\|+\|\|\| +\|\|+\|+\|+ \|+\| \| \| \|\|\|\|+\|\|+ | | |
| ENDO2A | 46 | SEMLDLKGKAKWEAWNPQKGLSTEDMMRAFISKAEELIEKYG 87 | | |

ENDO3

An ENDO3 nucleic acid of the invention includes the nucleic acid sequence shown in Table 12 (SEQ ID NO:5). An ORF, as well as putative untranslated regions upstream from the initiation codon and downstream from the stop codon of the ORF, are shown in Table 12 by underlining. The start and stop codons of the ORF are shown in bold letters. The ORF begins with an ATG initiation codon at nucleotides 86-88 and ends with a TGA codon at nucleotide 403-405. Putative untranslated regions upstream from the initiation codon and downstream from the stop codon are shown in Table 12 by underlining, whereas the start and stop codons are shown in bold letters.

TABLE 12

| | |
|---|---|
| GCTCACACCTGTAATCCCAGCATTTGGGAGGCCAAGGCAGGCAGATTATGTGAGGTCAAGAGT | (SEQ ID NO:5) |
| TCCAGACCAGCTGTCCAACATGGCAAAACCCATCTCCACTAAAAATACAAAAATTAGCCGGCA | |
| TGGGTGGCATGCAGCTGTAATCACAGCTGCTCGGGAGGCTGAGGCGGAGAATCACTTGAGCTG | |
| GGAAGAAAAAAAAAAAAAAAAAAGATGTGCAGGTATTAAGCACTTTAAGACCAAGCCAGCAGA | |
| TGATGAGATGCGGTTCCTTTACGGCCACTACAAACGAGCGACTGTAGGCAACATAAAGACAGA | |
| ACGGCCAGGGATGGTGGACTTCAAGGGCAAAGCCAAGTGGGATCCCTGGAATTTAGTGAAAGG | |
| GGCTGCCAGGGAAGATCCCATGAAAGCTAAAGCTTACGTCAAAAAAGTAGAAGAGTTAAAGAA | |
| AAAATTCAGAATACGAGAGACTGGAATTGTTGCCAGCCATGCCTTTGTCCTAAACTGAGACAA | |
| TGCCTTGTTTTTTCTACACTGTGGATGGTGGGAACTGATGGAAAGAATCAGCTAACCCATC | |

The disclosed ENDO3 nucleic acid sequence (SEQ ID NO:5) has 168 of 199 bases (84%) identical to a sequence on human chromosome 16 incorporated into bacterial artificial chromosome 462G18 (LANL) (GENBANK-ID: AC005736|acc:AC005736).

The ORF identified in Table 12 encodes a polypeptide sequence of 83 residues (SEQ ID NO:6), which is presented in Table 13.

TABLE 13

| | |
|---|---|
| MAKPISTKNTKISRHGWHAAVITAAREAEAENHLSWEEKKKKKRCAGIKHFKTKPADDEMRFL | (SEQ ID NO:6) |
| YGHYKRATVGNIKTERPGMVDFKGKAKWDPWNLVKGAAREDPMKAKAYVKKVEELKKKFRIRE | |
| TGIVASHAFVLN | |

The amino acid sequence of the polypeptide sequence disclosed in Table 13 (SEQ ID NO:6) has 55 of 82 amino acid residues (67%) identical to, and 66 of 82 residues (80%) positive with, the 86 amino acid residue bovine acyl-coA-binding protein (ACBP) (diazepam binding inhibitor)(DBI) (endozepine) (EP) (ptnr: SWISSPROT-ACC: P07107). A comparison of these sequences is presented in Table 14. Regions of the polypeptide of Table 13 are presented as the “Query” sequence, and regions of the bovine ACBP sequence are presented as the “Sbjct” sequence.

TABLE 14

| | | | | |
|---|---|---|---|---|
| Query: | 209 | KRCAGIKHFKTKPADDEMRFLYGHYKRATVGNIKTERPGMVDFKGKAKWDPWNLVKGARR | 388 | (SEQ ID NO:58) |
| | | \| +\|\| \|\|\|\|\|\|+\|\| \|+\| \|\|\|+\|\|\|\|+\| \|\|\|\|\|\|+\|\|\|\|\|\|\|\|\| \|\| +\|\| ++ | | |
| Sbjct: | 7 | KAAEEVKHLKTKPADEEMLFIYSHYKQATVGDINTERPGMLDFKGKAKWDAWNELKGTSK | 66 | (SEQ ID NO:59) |
| Query: | 389 | EDPMKAKAYVKKVEELKKKFRI 454 | | |
| | | \|\| \|\|\| \|+ \|\|\|\|\|\|\|\|+ \| | | |
| Sbjct: | 67 | EDAMKA--YIDKVEELKKKYGI 86 | | |

The amino acid sequence of the polypeptide sequence disclosed in Table 13 (SEQ ID NO:6) has 57 of 91 amino acid residues (62%) identical to, and 72 of 91 residues (79%) positive with 91 amino acid residues of Human diazepam binding inhibitor (DBI) (gb:GENBANK-ID:HUMDBI|acc:M14200)

A comparison of these sequences is presented in Table 14A. Regions of the polypeptide of Table 13 are presented as the “Query” sequence, and regions of the Human diazepam binding inhibitor (DBI) sequence are presented as the “Sbjct” sequence.

TABLE 14A

>gb:GENBANK-ID:HUMDBI|acc:M14200 Human diazepam binding inhibitor (DBI)
mRNA, complete cds—Homo sapiens, 556 bp.
Length = 556
Plus Strand HSPs:
Score = 310 (109.1 bits), Expect = 3.4e-26, P = 3.4e-26
Identities = 57 91 (62%), Positives = 72 91 (79%) , Frame = +2

| | | | | |
|---|---|---|---|---|
| Query: | 38 | EKKKKKRCAGIKHFKTKPADDEMRFLYGHYKRATVGNIKTERPGMVDFKGKAKWDPWNLV | 97 | (SEQ ID NO:60) |
| | | \| + +\| ++\| \|\|\|\|+\|+\|\| \|+\|\|\|\|\|+\|\|\|\|+\| \|\|\|\|\|\|+\|\| \|\|\|\|\|\| \|\| + | | |
| Sbjct: | 77 | EAEFEKAAEEVRHLKTKPSDEEMLFIYGHYKQATVGDINTERPGMLDFTGKAKWDAWNEL | 256 | (SEQ ID NO:61) |
| Query: | 98 | KGAAREDPMKAKAYVKKVEELKKKFRIRETG 128 | | |
| | | \|\| ++\|\| \|\|\| \|+ \|\|\|\|\|\|\|\|+ \| \|\|\| | | |
| Sbjct: | 257 | KGTSKEDAMKA--YINKVEELKKKYGI*ETG 343 | | |

A multiple sequence alignment illustrating the relatedness of the polypeptide disclosed in Table 13 to bovine and human Acyl co-A binding proteins is presented in Table 15 as a ClustalW analysis. Compared are the polypeptide of Table 12 (“DBI_novel”), bovine acyl coA-binding protein (SWISSPROT locus ACBP_BOVIN, accession P07107) (“ACBP_bovin”), and ACBP_human SWISSPROT locus ACBP_HUMAN, accession_P07108 (“ACBP_Human”). Regions of perfect homology are shown in black. Regions with conservative amino acid substitutions are shown in gray. Non-conservative amino acid substitutions are presented without shading.

[image]

Table 15-1 lists the sequence identifiers and sequence identification numbers (SEQ ID NO) for the sequences displayed in Table 15.

TABLE 15-1

| SEQUENCE IDENTIFIER | SEQ ID NO |
|---|---|
| DBI_novel | SEQ ID NO: 62 |
| ACBP_BOVIN | SEQ ID NO: 63 |
| ACBP_HUMAN | SEQ ID NO: 64 |

An ENDO3 nucleic acid of the invention includes a nucleic acid encoding a polyeptide comprising SEQ ID NO:6, e.g., a nucleic acid whose sequence is shown in SEQ ID NO:5. The invention also includes a fragment of the nucleic acid of SEQ ID NO:5, as well as a mutant or variant nucleic acid, any of whose bases may be changed from the corresponding base shown in Table 12, while still encoding a protein that maintains its endozepine-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, but are not limited to: modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

An ENDO3 protein of the invention includes the amino acid sequence shown in Table 13 (SEQ ID NO:6). The invention also includes a mutant or variant protein any of whose residues may be changed from the corresponding residue shown in Table 13, while still encoding a protein that maintains its endozepine-like activities and physiological functions, or a functional fragment thereof such as the following active peptides

Metabolism-Regulating Peptide #3 (MRP-3, 3s) Sequences (SEQ ID NO:17) and:

(SEQ ID NO:18)

| | |
|---|---|
| RATVGNIKTERPGMVDFKGK | (SEQ ID NO:17) |
| RATVGNIKTERPGMVDFK | (SEQ ID NO:18) |

The invention further encompasses antibodies and antibody fragments, such as F_{ab} or (F_{ab})₂, that bind immunospecifically to the ENDO3 polypeptide, and derivatives and fragments, thereof.

An ENDO3 sequence is useful for detecting specific types of tissue. For example when a panel of tissue is assayed for expression, ENDO3 is highly expressed in adipose and skeletal muscle. Also, high expression of ENDO3 is a marker for breast cancer.

An ENDO3 sequence is also useful to modulate global energy metabolism or weight by altering serum insulin or adipose level.

An ENDO3 sequence is also useful in a method to identity the cellular receptors and downstream effectors of the invention by any one of a number of techniques commonly employed in the art.

An ENDO3 sequence is useful in the treatment of diabetes, metabolic disturbances associated with obesity, the metabolic syndrome X as well as anorexia and wasting disorders associated with chronic diseases and various cancers by modulating metabolism.

ENDO4

An ENDO4 nucleic acid of the invention includes the nucleic acid sequence shown in Table 16 (SEQ ID NO:7). The sequence shown in Table 16 includes an ORF, as well as putative untranslated regions upstream and downstream from the ORF. The ORF begins with an atg initiation codon at nucleotides 11-13 and ends with a tga codon at nucleotides 299-301. The putative upstream and downstream untranslated regions are shown by underlining in Table 16.

TABLE 16

| | |
|---|---|
| TTGGTGGTAAATGCTCCTTTTGTTTGTTTGTTTGTTCTTCCTTAAGGCTGATTTTGACAGGGC | (SEQ ID NO:7) |
| TGCAGAAGATGTGAGGAAGCTGAAAGCAAGACCAGATGATGGAGAACTGAAAGAACTCTATGG | |
| GCTTTACAAACAAGCAATAGTTGGAGACATTAATATTGCGTGTCCAGGAATGCTAGATTTAAA | |
| AGGCAAAGCCAAATGGGAAGCATGGAACCTCAAAAAAGGGTTGTCGACGGAAGATGCGACGAG | |
| TGCCTATATTTCTAAAGCAAAGGAGCTGATAGAAAAATACGGAATTTAGAATACAGCA | |

The disclosed nucleic acid sequence (SEQ ID NO:7) has 200 of 256 bases (78%) identical to a Rana ridibunda endozepine mRNA (GENBANK-ID: RRU09205|acc:U09205). The relationship between the sequence of Table 16 and the Rana ridibunda sequence is presented in Table 17. Regions of the sequence shown in Table 16 are listed as the “Query” sequence. Regions of the Rana ridibunda endozepine mRNA sequence are shown as the “Subject” sequence.

TABLE 17

| | | | | |
|---|---|---|---|---|
| Query: | 45 | AGGCTGATTTTGACAGGGCTGCAGAAGATGTGAGGAAGCTGAAAGCAAGACCAGATGATG | 104 | (SEQ ID NO:65) |
| | | \|\|\|\| \|\|\|\|\|\|\|\|\|\| \|\| \|\|\|\| \|\|\|\|\| \| \|\|\| \|\|\|\|\| \|\|\| \|\|\|\| \|\|\| \| | | |
| Sbjct: | 23 | AGGCAGATTTTGACAAAGCAGCAGGGGATGTAAAGAAATTGAAAACAAAACCAACTGACG 82 | (SEQ ID NO:66) | |
| Query: | 105 | GAGAACTGAAAGAACTCTATGGGCTTTACAAACAAGCAATAGTTGGAGACATTAATATTG | 164 | |
| | | \|\|\|\|\|\|\|\| \|\|\|\|\| \|\| \|\| \|\| \|\|\|\|\| \|\| \| \| \|\|\|\|\| \|\|\|\|\| \|\|\|\|\| \| | | |
| Sbjct: | 83 | ATGAACTGAAGGAACTGTACGGACTCTACAAGCAGTCCACTGTTGGGGACATAAATATAG | 142 | |
| Query: | 165 | CGTGTCCAGGAATGCTAGATTTAAAAGGCAAAGCCAAATGGGAAGCATGGAACCTCAAAA | 224 | |
| | | \|\|\|\|\|\| \|\| \|\|\|\|\|\|\|\|\| \| \|\| \|\|\|\|\| \|\|\|\|\| \|\|\|\|\| \|\|\|\|\|\|\|\|\|\|\| \|\| \| | | |
| Sbjct: | 143 | AGTGTCCTGGCATGCTAGATCTGAAGGGCAAGGCCAAGTGGGACGCATGGAACCTAAAGA | 202 | |
| Query: | 225 | AAGGGTTGTCGACGGAAGATGCGACGAGTGCCTATATTTCTAAAGCAAAGGAGCTGATAG | 284 | |
| | | \|\|\|\| \|\|\|\|\| \| \|\|\|\|\|\|\|\|\|\|\| \|\|\| \|\| \|\|\| \|\|\|\|\|\|\|\|\|\| \| \|\|\|\|\|\|\|\|\|\| | | |
| Sbjct: | 203 | AAGGCTTGTCTAAGGAAGATGCGATGAGCGCTTATGTTTCTAAAGCCCATGAGCTGATAG | 262 | |
| Query: | 285 | AAAAATACGGAATTTA 300 | | |
| | | \|\|\|\|\|\|\| \|\| \| \|\| | | |
| Sbjct: | 263 | AAAAATATGGCCTGTA 278 | | |

The disclosed nucleic acid sequence (SEQ ID NO:7) is also related to a human diazepam binding inhibitor mRNA (GENBANK-ID:HUMDBI|acc:M14200). The disclosed sequence is identical at 179 of 259 residues (69%) to the human diazepam inhibitor mRNA. The relationship between the disclosed and the human sequence is presented in Table 18. Regions of the sequence shown in Table 16 are listed as the “Query” sequence. Regions of the human mRNA sequence are shown as the “Subject” sequence.

TABLE 18

| | | | | |
|---|---|---|---|---|
| Query: | 45 | AGGCTGATTTTGACAGGGCTGCAGAAGATGTGAGGAAGCTGAA-AGCAAGACCAGATGAT | 103 | (SEQ ID NO:67) |
| | | \|\|\|\|\|\|\| \|\|\|\|\| \| \|\|\|\|\|\|\|\| \|\| \|\| \|\|\| \| \|\| \|\| \| \|\|\|\| \|\|\| \|\|\| | | |
| Sbjct: | 78 | AGGCTGAGTTTGAGAAAGCTGCAGAGGAGGTTAGGCACCTTAAGACCAAG-CCATCGGAT | 136 | (SEQ ID NO:68) |
| Query: | 104 | GGAGAACTGAAAGAAC-TCTATGGGCTTTACAAACAAGCAATAGTTGGAGACATTAATAT | 162 | |
| | | \| \|\| \|\| \| \| \|\|\|\|\|\|\| \| \|\|\|\|\|\|\|\|\|\|\|\|\| \|\| \|\| \|\|\|\|\| \|\|\|\| | | |
| Sbjct: | 137 | GAGGAGATGCT-GTTCATCTATGGCCACTACAAACAAGCAACTGTGGGCGACATAAATAC | 195 | |
| Query: | 163 | TGCGTGTCCAGGAATGCTAGATTTAGGCAAAGCCAAATGGGAAGCATGGAACCTCAA | 222 | |
| | | \| \| \|\| \|\| \|\|\| \| \|\| \|\| \| \|\|\|\|\| \|\|\|\|\| \|\|\|\|\| \|\| \|\|\|\|\| | | |
| Sbjct: | 196 | AGAACGGCCCGGGATGTTGGACTTCACGGGCAAGGCCAAGTGGGATGCCTGGAATGAGCT | 255 | |
| Query: | 223 | AAAAGGGTTGTCGACGGAAGATGCGACGAGTGCCTATATTTCTAAAGCAAAGGAGCTGAT | 282 | |
| | | \|\|\|\|\|\| \|\| \| \|\|\|\|\|\|\|\|\| \| \|\| \|\| \|\| \|\| \|\|\|\| \| \| \|\|\|\|\| \| | | |
| Sbjc\|: | 256 | GAAAGGGACTTCCAAGGAAGATGCCATGAAAGCTTACATCAACAAAGTAGAAGAGCTAA- | 314 | |
| Query: | 283 | AGAAAAA-TACGGAATTT-AGA 302 | | |
| | | \|\|\|\|\|\|\| \|\|\|\|\| \|\| \| \|\|\| | | |
| Sbjct: | 315 | AGAAAAAATACGGGATATGAGA 336 | | |

The ORF identified in Table 16 encodes an amino acid sequence of 89 amino acids (SEQ ID NO:8). The amino acid sequence of the encoded protein (SEQ ID NO:8) is shown in Table 19.

TABLE 19

| | |
|---|---|
| MLLLFVCLFFLKADFDRAAEDVRKLKARPDDGELKELYGLYKQATVGDINIACPGMLDLKGKA | (SEQ ID NO:8) |
| KWEAWNLKKGLSTEDATSAYISKAKELIEKYGI | |

A signal peptide is present in the polypeptide sequence shown in Table 19. The most likely cleavage site between residues 18 and 19, at the sequence DRA-AE. The program PSORT predicts a moderate likelihood of extracellular secretion for the ENDO4 protein.

The polypeptide shown in Table 19 is related to previously described acyl-coA binding proteins and diazepam binding inhibitor proteins. Table 20 shows that the amino acid sequence of Table 19 has 71 of 89 amino acid residues (79%) identical to, and 78 of 89 residues (87%) positive with, the 103 amino acid residue protein from Anas platyrhynchos (ptnr: SWISSPROT-ACC:P45882). Regions of the polypeptide sequence shown in Table 19 are presented as the “Query” sequence. Regions of the Anas platyrhynchos sequence are shown as the “Sbjct” sequence.

TABLE 20

| | | | | |
|---|---|---|---|---|
| Query: | 35 | FFL-KADFDRAAEDVRKLKARPDDGELKELYGLYKQAIVGDINIACPGMLDLKGKAKWEA | 211 | (SEQ ID NO:69) |
| | | \|\|\| +\|\|\|\| \|\|\|+\|+\|\|\| \|\| \| \|\|\|\|\|\|\| \|\|\|\| \|\|\|\|\|\| \|\|\|\|\|\|\|\|\|\|\|\|\|\|\| | | |
| Sbjct: | 15 | FFLHQADFDEAAEEVKKLKTRPTDEELKELYGFYKQATVGDINIECPGMLDLKGKAKWEA | 74 | (SEQ ID NO:70) |
| Query: | 212 | WNLKKGLSTEDATSAYISKAKELIEKYGI 298 | | |
| | | \|\|\|\|\|\|+\| \|\|\| +\|\|\|\|\|\|\| ++\|\|\|\|\| | | |
| Sbjct: | 75 | WNLKKGISKEDAMNAYISKAKTMVEKYGI 103 | | |

Table 20A shows that the amino acid sequence of Table 19 has homology to Rana ridibunda diazepam-binding inhibitor (DBI). Regions of the polypeptide sequence shown in Table 19 are presented as the “Query” sequence. Regions of the Rana ridibunda diazepam-binding inhibitor (DBI) sequence are shown as the “Sbjct” sequence.

TABLE 20A

>gb GENBANK-ID:RRU09205|acc:U09205 Rana ridibunda diazepam-binding inhibitor
(DBI) mRNA, complete cds—Rana ridibunda, 470 bp.
Length = 470
Plus Strand HSPs:
Score = 365 (128.5 bits), Expect = 6.0e-32, P = 6.0e-32
Identities = 68 85 (80%), Positives = 76 85 (89%), Frame = +1

| | | | | |
|---|---|---|---|---|
| Query: | 12 | KADFDRAAEDVRKLKARPDDGELKELYGLYKQAIVGDINIACPGMLDLKGKAKWEAWNLK | 71 | (SEQ ID NO:71) |
| | | +ADFD+AA DV+KLK +P D ELKELYGLYKQ+ VGDINI CPGMLDLKGKAKW+AWNLK | | |
| Sbjct: | 22 | QADFDKAAGDVKKLKTKPTDDELKELYGLYKQSTVGDINIECPGMLDLKGKAKWDAWNLK | 201 | (SEQ ID NO:73) |
| Query: | 72 | KGLSTEDATSAYISKAKELIEKYGI 96 | | |
| | | KGLS EDA SAY+SKA ELIEKYG+ | (SEQ ID NO:72) | |
| Sbjct: | 202 | KGLSKEDAISAYVSKAflELIEKYGL 276 | | |

An alignment between the polypeptide sequence shown in Table 18 and previously described diazepam binding inhibitor or Acyl-coA binding polypeptides is shown in Table 21. The amino acid sequence shown in Table 19 is presented as “ba271m1_A”. An 88 amino acid frog acyl co-A binding protein amino acid sequence (PIR-ID: A57711) is indicated by “A57711_ACBP_Frog.: An 88 amino acid human acyl co-A binding polypeptide (PIR-ID: NZHU) sequence is shown by “NZHU_ACBP_Human”. A 103 amino acid duck endozepine amino acid sequence (SWISSPROT-ACC:_{—}45882) is indicated by “P45882_endozepine_Duck”. Regions with conservative amino acid substitutions are shown in gray. Non-conservative amino acid substitutions are presented without shading.

[image]

Table 21-1 lists the sequence identifiers and sequence identification numbers (SEQ ID NO) for the sequences displayed in Table 21.

TABLE 21-1

| SEQUENCE IDENTIFIER | SEQ ID NO |
|---|---|
| ba271m1_A | SEQ ID NO: 74 |
| A57711_ACBP_Frog | SEQ ID NO: 75 |
| P45882_Endezopine_Duck | SEQ ID NO: 76 |
| NZHU_ACBP_Human | SEQ ID NO: 77 |

An ENDO4 nucleic acid of the invention includes a nucleic acid encoding a polypeptide that includes the amino acid sequence of SEQ ID NO:8. For example, an ENDO4 nucleic acid can include the sequence disclosed in Table 16 (SEQ ID NO:7). The invention alco includes fragments of a nucleic acid encoding a polypeptide that includes the amino acid sequence of SEQ ID NO:8. The invention also includes a mutant or variant nucleic acid any of whose bases may be changed from the corresponding base shown in Table 16, while still encoding a protein that maintains its endozepine-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, but are not limited to: modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

An ENDO4 protein of the invention includes the protein whose amino acid sequence is shown in Table 19 (SEQ ID NO:8). The invention also includes a mutant or variant protein any of whose residues may be changed from the corresponding residue shown in Table 19, while still encoding a protein that maintains its endozepine-like activities and physiological functions, or a functional fragment thereof such as the following active peptides

Metabolism-Regulating Peptide #4 (MRP-4,4s) Sequences:

| | |
|---|---|
| QAIVGDINIACPGMLDLKGK | (SEQ ID NO:19) |
| QAIVGDINIACPGMLDLK | (SEQ ID NO:20) |

The invention further encompasses antibodies and antibody fragments, such as F_{ab} or (F_{ab})₂, that bind immunospecifically to the ENDO4 polypeptide, and derivatives and fragments, thereof.

An ENDO4 sequence is useful for detecting specific types of tissue. For example when a panel of tissue is assayed for expression, ENDO4 is highly expressed in hematopoietic tissue.

An ENDO4 sequence is also useful to modulate global energy metabolism or weight by altering serum insulin and glucose.

An ENDO4 sequence is also useful in a method to identity the cellular receptors and downstream effectors of the invention by any one of a number of techniques commonly employed in the art.

An ENDO4 sequence is useful in the treatment of diabetes, metabolic disturbances associated with obesity, the metabolic syndrome X as well as anorexia and wasting disorders associated with chronic diseases and various cancers by modulating metabolism.

ENDO4A

ENDO4A is a variant of ENDO4. The ENDO4A nucleic acid of 297 nucleotides (also referred to as CG55148-O₂) (SEQ ID NO:178) encoding a novel ACYL-COA-BINDING PROTEIN (ACBP)-like protein is shown in Table 21A. An open reading frame was identified beginning at nucleotides 3-5 and ending at nucleotides 264-266. The start and stop codons of the open reading frame are highlighted in bold type. Putative untranslated regions (underlined), if any, are found upstream from the initiation codon and downstream from the termination codon.

TABLE 21A

| | | |
|---|---|---|
| TCTTCCTTAAGGCTGATTTTGACAGGGCTGCAGAAGATGTGAGGAAGCTGAAAGCAAGAC | 60 | (SEQ ID NO:178) |
| CAGATGATGGAGAACTGAAAGAACTCTATGGGCTTTACAAACAAGCAATAGTTGGAGACA | 120 | |
| TTAATATTGCGTGTCCAGGAATGCTAGATTTAAAAGGCAAAGCCAAATGGGAAGCATGGA | 180 | |
| ACCTCAAAAAAGGGTTGTCGACGGAAGATGCGACGAGTGCCTATATTTCTAAAGCAAAGG | 240 | |
| AGCTGATAGAAAAATACGGAATTTAGAATACAGCATATGAGGAATTTTTCCTTTTGA | 297 | |

In a search of sequence databases, it was found, for example, that the nucleic acid sequence of ENDO4A has 200 of 256 bases (78%) identical to a gb:GENBANK-ID:RRU09205|acc:U09205.1 mRNA from Rana ridibunda (Rana ridibunda diazepam-binding inhibitor (DBI) mRNA, complete cds).

The ENDO4A protein having 87 amino acid residues (SEQ ID NO:179) is presented using the one-letter code in Table 21B. Although PSORT suggests that ENDO4A may be localized in the cytoplasm with a certainty of 0.6500, the ENDO4A protein is similar to the ACYL-COA-BINDING PROTEIN family, some members of which are secreted. Therefore it is likely that this novel ACYL-COA-BINDING PROTEIN (ACBP)-like protein is likely to be an extracellular protein. In alternative embodiments, ENDO4A is likely to be localized to the mitochondrial matrix space with a certainty of 0.1000, to the lysosome lumen with a certainty of 0.1000, or to the microbody (peroxisome) with a certainty of 0.0299.

TABLE 21B

| | | |
|---|---|---|
| FLKADFDRAAEDVRKLKARPDDGELKELYGLYKQAIVGDINIACPGMLDLKGKAKWEAWN | 60 | (SEQ ID NO:179) |
| LKKGLSTEDATSAYISKAKELIEKYGI 87 | | |

The full amino acid sequence of ENDO4A was found to have 70 of 88 amino acid residues (79%) identical to, and 77 of 88 amino acid residues (87%) similar to, the 103 amino acid residue ptnr:SWISSPROT-ACC:P45882 protein from Anas platyrhynchos (Domestic duck) (ACYL-COA-BINDING PROTEIN (ACBP) (DIAZEPAM BINDING INHIBITOR) (DBI) (ENDOZEPINE) (EP)).

In a further search of public sequence databases, ENDO4A was found to have homology to the amino acid sequences shown in the BLASTP data listed in Table 21C.

TABLE 21C
BLASTP results for ENDO4A

| Gene Index/ | | Length | Identity | Positives | |
|---|---|---|---|---|---|
| Identifier | Protien/Organism | (aa) | (%) | (%) | Expect |
| SPTREMBL- | 9230116B18RIK PROTEIN - | 88 | 70/86 | 80/86 | 1.0e−33 |
| ACC: Q9D258 | Mus musculus | | (81%) | (93%) | |
| SWISSPROT- | Acyl-CoA-binding | 103 | 70/88 | 77/88 | 2.7e−33 |
| ACC: P45882 | protein (ACBP) | | (79%) | (87%) | |
| | (Diazepam binding inhibitor) | | | | |
| | (DBI) (Endozepine) (EP) - | | | | |
| | Anas platyrhynchos | | | | |
| SWISSPROT- | Acyl-CoA-binding | 87 | 68/85 | 76/85 | 3.4e−33 |
| ACC: P45883 | protein homolog (ACBP) | | (80%) | (89%) | |
| | (Diazepam binding | | | | |
| | inhibitor homolog) | | | | |
| | (DBI) - Rana ridibunda | | | | |
| pir-id: A57711 | diazepam-binding | 88 | 68/85 | 76/85 | 3.4e−33 |
| | inhibitor - laughing frog | | (80%) | (89%) | |
| SWISSPROT- | Acyl-CoA-binding | 86 | 52/85 | 64/85 | 3.1e−23 |
| ACC: P07108 | protein (ACBP) | | (61%) | (75%) | |
| | (Diazepam binding inhibitor) | | | | |
| | (DBI) (Endozepine) (EP) - | | | | |
| | Homo sapiens | | | | |

A multiple sequence alignment is given in Table 21D in a ClustalW analysis comparing ENDO1A with related protein sequences shown in Table 21C.

[image]

BLAST analysis was performed on sequences from the Patp database, which is a proprietary database that contains sequences published in patents and patent publications. Patp results include those listed in Table 21E.

TABLE 21E
Patp BLASTP Analysis for ENDO4A

| Sequences | | | | | |
|---|---|---|---|---|---|
| producing High- | | | | | |
| scoring Segment | | Length | Identity | Positive | |
| Pairs | Protein/Organism | (aa) | (%) | (%) | E Value |
| AAB81811 | Human endozepine-like | 96 | 87/87 | 87/87 | 2.8e−42 |
| | ENDO4 SEQ ID NO: 8 - Homo sapiens | | (100%) | (100%) | |
| AAB81806 | Human endozepine-like | 88 | 64/86 | 72/86 | 2.4e−29 |
| | ENDO2 SEQ ID NO: 4 - Homo sapiens | | (74%) | (83%) | |
| AAE09445 | Human sbg35069DBIa | 88 | 64/86 | 72/86 | 2.4e−29 |
| | protein - Homo sapiens | | (74%) | (83%) | |
| AAG78314 | Human DBI/ACBP-like | 88 | 64/86 | 72/86 | 2.4e−29 |
| | protein - Homo sapiens | | (74%) | (83%) | |
| AAB81814 | Human endozepine-like | 86 | 60/84 | 67/84 | 7.6e−24 |
| | ENDO5 SEQ ID NO: 10 - Homo sapiens | | (71%) | (79%) | |

The presence of identifiable domains in the protein disclosed herein was determined by searches versus domain databases such as Pfam, PROSITE, ProDom, Blocks or Prints and then identified by the Interpro domain accession number. Significant domains are summarized in Table 21F.

TABLE 21F

| Model | Domain | seq-f | seq-t | hmm-f | hmm-t | score | E-value |
|---|---|---|---|---|---|---|---|
| -------- | ------- | ----- | ----- | ----- | ----- | ----- | ------- |
| ACBP | 1/1 | 2 | 86 | 1 | 89 | 165.2 | 1.1e-45 |

Alignments of top-scoring domains:
ACBP: domain 1 of 1, from 2 to 86: score 165.2, E=1.1e-45

| | | | | |
|---|---|---|---|---|
| | | lqedFeaAaekvkkLkknGpvkpSneekLkLYsLYKoATvGDvnter | | (SEQ ID NO:180) |
| | | \|++\|\|+ \|\|\| \|+\|\|\| + \|++ \|+ +\|\|+\|\|\|\|\| \|\|\|+\|+ + | | |
| ENDO4A2 | 2 | LKADFDRAAEDVRKLKAR - - - - PDDGELKELYGLYKQAIVGDINIAC | 44 | (SEQ ID NO:181) |
| | | PGmfDlkgrAKWDAWnelkGmSkeeAmkaYlakVeeLiakya | | |
| | | \|\|\|+\|\|\|\|+\|\|\|+\|\|\| +\|\|+\|+\|+\| +\|\|\| \| +\|\|\|+\|\|+ | | |
| ENDO4A | 45 | PGMLDLKGKAKWEAWNLKKGLSTEDATSAYISKAKELIEKYG 86 | | |

Acyl-CoA-binding protein (ACBP) is a small (10 Kd) protein that binds medium- and long-chain acyl-CoA esters with high affinity, and may act as an intra-cellular carrier of acyl-CoA esters. ACBP has a number of important physiological and biochemical functions: it is known as a diazepam binding inhibitor, as a putative neurotransmitter, as a regulator of insulin release from pancreatic cells, and as a mediator in corticotropin-dependent adrenal steroidogenesis. It is possible that the protein acts as a neuropeptide that takes part in the modulation of gamma-aminobutyric acid-ergic transmission. The structure of ACBP has been deduced by NMR spectroscopy and has been shown to be a mainly-alpha protein, consisting of 5 short alpha-helices and 3 connecting beta-strands.

ACBP is a highly conserved protein of about 90 residues that has been so far found in vertebrates, insects, plants and yeast. Other proteins belonging to the ACBP family include mouse endozepine-like peptide (ELP) (gene DBIL5); mammalian MA-DBI, a transmembrane protein of unknown function which has been found in mammals; and human DRS-1, a protein of unknown function that contains a N-terminal ACBP-like domain and a C-terminal enoyl-CoA isomerase/hydratase domain.

This indicates that the sequence of the invention has properties similar to those of other proteins known to contain this/these domain(s) and similar to the properties of these domains.

The ACYL-COA-BINDING PROTEIN (ACBP)-like ENDO4A disclosed in this invention is expressed in at least the following tissues: Mammalian Tissue, Substantia Nigra, and Hippocampus.

The nucleic acids and proteins of the invention have applications in the diagnosis and/or treatment of various diseases and disorders. For example, the compositions of the present invention will have efficacy for the treatment of patients suffering from: Anxiety disorders; CNS disorders where GABA neuro-transmitters are involved; Diabetes and Obesity as well as other diseases, disorders and conditions.

These materials are further useful in the generation of antibodies that bind immunospecifically to the novel substances of the invention for use in diagnostic and/or therapeutic methods.

ENDO5

An ENDO5 nucleic acid according to the invention includes the nucleic acid sequence shown in Table 25 (SEQ ID NO:9). An ORF is present in the disclosed sequence, as well as putative untranslated regions upstream and downstream of the ORF. The ORF begins with an atg initiation codon at nucleotides 7-9 and ends with a tag codon at nucleotides 265-267. The putative upstream and downstream untranslated regions are shown by underlining in Table 22.

TABLE 22

| | |
|---|---|
| ACCACCATGGCACTGCAGGCTGAATTCGACAAGGCTGCAGAAGACGTGAGGAAGCTGCCAACA | (SEQ ID NO:9) |
| AGACCAGCAGATAATAAAGAACTGAAAAAACTCGATGGACTTTACAAACAAGCTATAATTGGA | |
| GACATTAATATTGAGTATCTGGGAATGCTGGACTTTAAGGGCAAGGCCAAATGCGCAGCATGG | |
| ACCCTCCAAAAAAGGTTGTCAAAGGAAGATGCAACGAGTGTCTCTATTTCTAAGGCAAAAGAG | |
| CCGATAGAAAAATAGGACATTTAGAATA | |

The ENDO5 nucleic acid sequence (SEQ ID NO:9) has 199 of 274 nucleotides (72%) identical to a Rana ridibunda endozepine mRNA GENBANK-ID: RRU09205|acc:U09205. A comparison of these nucleotide sequences is shown in Table 23. The sequence disclosed in Table 22 is presented as the “Query” sequence, and the Rana ridibunda endozepine mRNA sequence is presented as as the “Sbjct” sequence.

TABLE 23

| | | | | |
|---|---|---|---|---|
| Query: | 2 | CCACCATGGCACTGCAGGCTGAATTCGACAAGGCTGCAGAAGACGTGAGGAAGCTGCCAA | 61 | (SEQ ID NO:78) |
| | | \| \|\|\|\|\|\| \|\|\| \|\|\|\|\| \|\| \|\| \|\|\|\|\| \|\| \|\|\|\| \|\| \|\| \| \|\|\| \|\| | | |
| Sbjct: | 8 | CAACCATGTCACCCCAGGCAGATTTTGACAAAGCAGCAGGGGATGTAAAGAAATTGAAAA | 67 | (SEQ ID NO:79) |
| Query: | 62 | CAAGACCAGCAGATAATAAAGAACTGAAAAAACTCGATGGACTTTACAAACAAGCTATAA | 121 | |
| | | \|\|\| \|\|\|\| \| \|\| \|\| \|\|\|\|\|\|\|\| \|\|\|\| \| \|\|\|\|\| \|\|\|\|\| \|\| \| \| | | |
| Sbjct: | 68 | CAAAACCAACTGACGAT---GAACTGAAGGAACTGTACGGACTCTACAAGCAGTCCACTG | 124 | |
| Query: | 122 | TTGGAGACATTAATATTGAGTATCTGGGAATGCTGGACTTTAAGGGCAAGCCCAAATGCG | 181 | |
| | | \|\|\|\| \|\|\|\|\| \|\|\|\|\| \|\|\|\| \|\| \|\| \|\|\|\|\| \|\| \| \|\|\|\|\|\|\|\|\|\|\|\|\|\| \|\| \| | | |
| Sbjct: | 125 | TTGGGGACATAAATATAGAGTGTCCTGGCATGCTAGATCTGAAGGGCAAGGCCAAGTGGG | 184 | |
| Query: | 182 | CAGCATGGACCCTCCAAAAAAGG-TTGTCAAAGGAAGATGCAACGAGTGTCTCTATTTCT | 240 | |
| | | \|\|\|\|\|\|\| \|\|\| \|\| \|\|\|\|\| \|\|\|\|\| \|\|\|\|\|\|\|\|\|\|\| \| \|\|\| \| \| \| \|\|\|\|\| | | |
| Sbjct: | 185 | ACGCATGGAACCTA-AAGAAAGGCTTGTCTAAGGAAGATGCGATGAGCGCTTATGTTTCT | 243 | |
| Query: | 241 | AAGGCAAAAGAGCCGATAGAAAAATAGGACATTTA 275 | | |
| | | \|\| \|\| \| \|\|\|\| \|\|\|\|\|\|\|\|\|\|\|\| \| \| \| \|\| | | |
| Sbjct: | 244 | AAAGCCCATGAGCTGATAGAAAAATATGGCCTGTA 278 | | |

The ENDO5 nucleic acid sequence (SEQ ID NO:9) also has 173 of 262 nucleotides (66%) identical to a Homo sapiens endozepine mRNA GENBANK-ID: HUMEDZ|acc:M15887. A comparison of these nucleotide sequences is shown in Table 24. The sequence disclosed in Table 22 is presented as the “Query” sequence, and the human endozepine mRNA sequence is presented as the “Sbjct” sequence.

TABLE 24

| | | | | |
|---|---|---|---|---|
| Query: | 16 | CAGGCTGAATTCGACAAGGCTGCAGAAGACGTGAGGAAGCTGCCAAC-AAGACCAGCAGA | 74 | (SEQ ID NO:80) |
| | | \|\|\|\|\|\|\|\| \|\| \|\| \|\| \|\|\|\|\|\|\|\| \|\| \|\| \|\|\| \|\|\| \|\| \|\|\| \|\|\| \| \|\| | | |
| Sbjct: | 64 | CAGGCTGAGTTTGAGAAAGCTGCAGAGGAGGTTAGGCACCTTAAGACCAAG-CCATCGGA | 122 | (SEQ ID NO:81) |
| Query: | 75 | TAATAAAGAA-CTGAAAAAACTCGATGGACTTTACAAACAAGCTATAATTGGAGACATTA | 133 | |
| | | \| \| \| \|\| \|\|\| \| \|\| \|\|\|\| \| \|\|\|\|\|\|\|\|\|\|\| \| \| \|\| \|\|\|\|\| \| | | |
| Sbjct: | 123 | TGAGGA-GATGCTGTTCAT-CT--ATGGCCACTACAAACAAGCAACTGTGGGCGACATAA | 178 | |
| Query: | 134 | ATATTGAGTATCTGGGAATGCTGGACTTTAAGGGCAAGGCCAAATGCGCAGCATGGACCC | 193 | |
| | | \|\|\| \|\| \| \|\| \|\|\| \|\|\|\|\|\|\| \| \|\|\|\|\|\|\|\|\|\|\|\| \|\| \| \|\| \|\|\|\| | | |
| Sbjct: | 179 | ATACAGAACGGCCCGGGATGTTGGACTTCACGGGCAAGGCCAAGTGGGATGCCTGGAATG | 238 | |
| Query: | 194 | TCCAAAAAAGGTTGTCAAAGGAAGATGCAACGAGTGTCTCTATTTCTAACGCAAAAGAGC | 253 | |
| | | \| \|\|\| \|\| \|\| \|\|\|\|\|\|\|\|\|\|\| \| \|\| \| \| \|\| \|\| \| \| \|\|\|\|\|\| | | |
| Sbjct: | 239 | AGCTGAAAGGGACTTCCAAGGAAGATGCCATGAAAGCTTACATCAACAAACTAGAAGAGC | 298 | |
| Query: | 254 | CGATAGAAAAA-TAGGACATTT-AGA 277 | | |
| | | \|\|\|\|\|\|\|\| \|\| \| \|\| \| \|\|\| | | |
| Sbjct: | 299 | TAA-AGAAAAAATACGGGATATGAGA 323 | | |

The ORF identified in Table 22 encodes a polypeptide of 86 amino acid residues (SEQ ID NO:10). The amino acid sequence of the encoded polypeptide is shown in Table 25.

TABLE 25

| | |
|---|---|
| MALQAEFDKAAEDVRKLPTRPADNKELKKLDGLYKQAIIGDINIEYLGMLDFKGKAKCAAWTL | (SEQ ID NO:10) |
| QKRLSKEDATSVSISKAKEPTEK | |

The encoded polypeptide (SEQ ID NO:10) shown in Table 25 has 57 of 86 amino acid residues (66%) identical to, and 67 of 86 residues (77%) positive with, the 88 amino acid residue diazepam binding inhibitor protein from Rana ridibunda (ptnr:PIR-ID:A57711). An alignment of these sequences is shown in Table 26. A comparison of these amino acid sequences is shown in Table 26. The sequence disclosed in Table 25 is presented as the “Query” sequence, and the Rana ridibunda endozepine polypeptide sequence is presented as the “Sbjct” sequence.

TABLE 26

| | | | | |
|---|---|---|---|---|
| Query: | 7 | MALQAEFDKAAEDVRKLPTRPADNKELKKLDGLYKQAIIGDINIEYLGMLDFKGKAKCAA | 186 | (SEQ ID NO:82) |
| | | + \|\|+\|\|\|\|\| \|\|+\|\| \|+\| \|+ \|\|\|+\| \|\|\|\|\|+ +\|\|\|\|\|\| \|\|\|\| \|\|\|\|\| \| | | |
| Sbjct: | 1 | MSPQADFDKAAGDVKKLKTKPTDD-ELKELYGLYKQSTVGDINIECPGMLDLKGKAKWDA | 59 | (SEQ ID NO:83) |
| Query: | 187 | WTLQKRLSKEDATSVSISKAKEPIEK 264 | | |
| | | \| \|+\| \|\|\|\|\|\| \| +\|\|\| \| \|\|\| | | |
| Sbjct: | 60 | WNLKKGLSKEDAMSAYVSKAHELIEK 85 | | |

An alignment showing the relatedness of the polypeptide sequence shown in Table 25 to previously described endozepine sequences is shown in Table 27. The 86 amino acid polypeptide of Table 25 is shown as “citb_e1_{—}2540m10_A”. The other endozepine polypeptide sequences present in the table include 88 amino acid sequence of frog diazepam binding inhibitor DBI (PIR-ID:A57711) (“A57711”), a 103 amino acid sequence duck polypeptide (SWISSPROT-ACC:P45882) (“P45882_Duck_DB1), and an 87amino acid human polypeptide (NZHU_Human_DBI). Regions with conservative amino acid substitutions are shown in gray. Non-conservative amino acid substitutions are presented without shading.

[image]

[image]

Table 27-1 lists the sequence identifiers and sequence identification numbers (SEQ ID NO) for the sequences displayed in Table 27.

TABLE 27-1

| SEQUENCE IDENTIFIER | SEQ ID NO |
|---|---|
| A57711_Frog_DBI | SEQ ID NO: 84 |
| P45882_Duck_DBI | SEQ ID NO: 85 |
| NZHU_Human_DBI | SEQ ID NO: 86 |
| Citb_e1_2540m10_A | SEQ ID NO: 87 |

Using the PSORT program, it is predicted that the disclosed ENDO5 protein localizes to the cytoplasm with a certainty of 0.6500. In an analysis using the SIGNALP program, it is predicted that the ENDO5 protein does not possess a signal peptide.

The invention includes an ENDO5 nucleic acid encoding a polypeptide that includes the amino acid sequence of SEQ ID NO:10, e.g., a nucleic acid including the nucleotide sequence of SEQ ID NO:9, or a fragment thereof. The invention also includes a mutant or variant nucleic acid, any one of whose bases may be changed from the corresponding base, as illustrated in Table 22, while still encoding a protein which maintains its endozepine-like activities and physiological functions, or a fragment of such a nucleic acid. The invention further includes nucleic acids whose sequences are complementary to those previously described, including nucleic acid fragments that are complementary to any of the nucleic acids previously described. The invention additionally includes nucleic acids or nucleic acid fragments, or complements thereto, whose structures include chemical modifications. Such modifications include, by way of non-limiting example, modified bases, and nucleic acids whose sugar-phosphate backbones are modified or derivatized. These modifications are carried out, at least in part, to enhance the chemical stability of the modified nucleic acid, such that they may be used, e.g., as antisense binding nucleic acids in therapeutic applications.

An ENDO5 protein of the invention includes the polypeptide (SEQ ID NO:10) whose sequence is illustrated in Table 25. The invention also includes a mutant or variant protein any of whose residues may be changed from the corresponding residue illustrated in Table 25, while still encoding a protein which maintains its endozepine-like activities and physiological functions, or a functional fragment thereof such as the following active peptide

Metabolism-Regulating Peptide #7 (MRP-7) Sequence:

1 QAIIGDINIEYLGMLDFKGK (SEQ ID NO:21)

The invention further encompasses antibodies and antibody fragments, such as F_{ab} or (F_{ab})₂ which immunospecifically-bind to the ENDO5 polypeptide, and derivatives and fragments, thereof.

An ENDO5 sequence is useful for detecting specific types of tissue. For example when a panel of tissue is assayed for expression, ENDO5 is highly expressed in adipose and hematopietic tissue.

An ENDO5 sequence is also useful to modulate global energy metabolism or weight by altering serum cholesterol or glucose.

An ENDO5 sequence is also useful in a method to identity the cellular receptors and downstream effectors of the invention by any one of a number of techniques commonly employed in the art.

An ENDO5 sequence is useful in the treatment of diabetes, metabolic disturbances associated with obesity, the metabolic syndrome X as well as anorexia and wasting disorders associated with chronic diseases and various cancers by modulating metabolism.

ENDO6

An ENDO6 nucleic acid of the invention includes the nucleic acid sequence shown in Table 27A (SEQ ID NO:22).

TABLE 27A

| | | |
|---|---|---|
| 1 | ATGTTCCAGTTTCATGCAGGCTCTTGGGAAAGCTGGTGCTGCTGCTGCCTGATTCCCGCCGACAGACCTTGGGACCGGGG | (SEQ ID NO:22) |
| 81 | CCAACACTGGCAGCTGGAGATGGCGGACACGAGATCCGTGCACGAGACTAGGTTTGAGGCGGCCGTGAAGGTGATCCAGA | |
| 161 | GTTTGCCGAAGAATGGTTCATTCCAGCCAACAAATGAAATGATGCTTAAATTTTATAGCTTCTATAAGCAGGCAACTGAA | |
| 241 | GGACCCTGTAAACTTTCAAGGCCTGGATTTTGGGATCCTATTGGAAGATATAAATGGGATGCTTGGAGTTCACTGGGTGA | |
| 321 | TATGACCAAAGAGGAAGCCATGATTGCATATGTTGAAGAAATGAAAAAGATTATTGAAACTATGCCAATGACTGAGAAAG | |
| 401 | TTGAAGAATTGCTGCGTGTCATAGGTCCATTTTATGAAATTGTCGAGGACAAAAAGAGTGGCAGGAGTTCTGATATAACC | |
| 481 | TCAGTCCGACTGGAGAAAATCTCTAAATGTTTAGAAGATCTTGGTAATGTTCTCACTTCTACTCCAAACGCCAAAACCGT | |
| 561 | TAATGGTAAAGCTGAAAGCAGTGACAGTGGAGCGGAGTCTGAGGAAGAAGAGGCCCAAGAAGAAGTGAAAGGAGCAGAAC | |
| 641 | ACAGTGATAATGATAAGAAAATGATGAAGAAGTCAGCAGACCATAAGAATTTGGAAGTCATTGTCACTAATGGCTATGAT | |
| 721 | AAAGATGGCTTTGTTCAGGATATACAGAATGACATTCATGCCAGTTCTTCCCTGAATGGCAGAAGCACTGAAGAAGTAAA | |
| 801 | GCCCATTGATGAAAACTTGGGGCAAACTGGAAAATCTGCTGTTTGCATTCACCAAGGTATTAATGATGATCATGTTGAAG | |
| 881 | ATGTTACAGGAATTCAGCATTTGACAAGCGATTCAGACAGTGAAGTTTACTGTGATTCTATGGAACAATTTGGACAAGAA | |
| 961 | GAGTCTTTAGACAGCTTTACGTCCAACAATGGACCATTTCAGTATTACTTGGGTGGTCATTCCAGTCAACCCATGGAAAA | |
| 1041 | TTCTGGATTTCGTGAAGATATTCAAGTACCTCCTGGAAATGGCAACATTGGGAATATGCAGGTGGTTGCAGTTGAAGGAA | |
| 1121 | AAGGTGAAGTCAAGCATGGAGGAGAAGATGGCAGGAATAACAGCGGAGCACCACACCGGGAGAAGCGAGGCGGAGAAACT | |
| 1201 | GACGAATTCTCTAATGTTAGAAGAGGAAGAGGTCATAGGATGCAACACTTGAGCGAAGGAACCAAGGGCCGGCAGGTGGG | |
| 1281 | AAGTGGAGGTGATGGGGAGCGCTGGGGCTCCGACAGAGGGTCCCGAGGCAGCCTCAATGAGCAGATCGCCCTCGTGCTGA | |
| 1361 | TGAGACTGCAGGAGGACATGCAGAATGTCCTTCAGAGACTGCAGAAACTGGAAACGCTGACTGCTGCAAAATCATCAACA | |
| 1441 | TCAACATTGCAGACTGCTCCTCAGCCCACCTCATCTCAGAGACCATCTTGGTGGCCCTTCGAGATGTCTCCTGGTGTGCT | |
| 1521 | AACGTTTGCCATCATATGGCCTTTTATTGCACAGTGGTTGGTGTATTTATACTATCAAAGAAGGAGAAGGTAA | |

The nucleic acid sequence disclosed in Table 27A includes an open reading frame (“ORF”) beginning at position 1 with start and stop codons indicated in bold. The ORF encodes a polypeptide sequence of 530 amino acid residues. The sequence of this encoded polypeptide (SEQ ID NO:23) is presented in Table 27B. The homology between the translated protein and Bovine endozepine (putativee ligand of benzodiazepine receptor) related protein (gb:GENBANK-ID:BOVEDZR|acc:M15888) is presented in Table 27C.

TABLE 27B

| | | |
|---|---|---|
| 1 | MFQFHAGSWESWCCCCLIPADRPWDRGQHWQLEMADTRSVHETRFEAAVKVIQSLPKNGSFQPTNEMMLKFYSFYKQATE | (SEQ ID NO:23) |
| 81 | GPCKLSRPGFWDPIGRYKWDAWSSLGDMTKEEAMIAYVEEMKKIIETMPMTEKVEELLRVIGPFYEIVEDKKSGRSSDIT | |
| 161 | SVRLEKISKCLEDLGNVLTSTPNAKTVNGKAESSDSGAESEEEEAQEEVKGAEHSDNDKKMMKKSADHKNLEVIVTNGYD | |
| 241 | KDGFVQDIQNDIHASSSLNGRSTEEVKPIDENLGQTGKSAVCIHQGINDDHVEDVTGIQHLTSDSDSEVYCDSMEQFGQE | |
| 321 | ESLDSFTSNNGPFQYYLGGHSSQPMENSGFREDIQVPPGNGNIGNMQVVAVEGKGEVKHGGEDGRNNSGAPHREKRGGET | |
| 401 | DEFSNVRRGRGHRMQHLSEGTKGRQVGSGGDGERWGSDRGSRGSLNEQIALVLMRLQEDMQNVLQRLQKLETLTAAKSST | |
| 481 | STLQTAPQPTSSQRPSWWPFEMSPGVLTFAIIQPFIAQWLVYLYYQRRRR | |

Translated Protein-Frame: 1-Nucleotide 1 to 1590

TABLE 27C

>gb:GENBANK-ID:BOVEDZR|acc:M15888 Bovine endozepine (putativee ligand of
benzodiazepine receptor) related protein mRNA, complete cds-Bos taurus,
1750 bp.
Length = 1750
Plus Strand HSPs:
Score = 2401 (845.2 bits), Expect = 2.9e−248, P = 2.9e−248
Identities = 448/530 (84%), Positives = 481/530 (90%), Frame = +2

| | | | | |
|---|---|---|---|---|
| Query: | 1 | MFQFHAGSWESWCCCC-LIPADRPWDRGQHWQLEMADTRSVHETRFEAAVKVIQSLPKNG | 59 | (SEQ ID NO:88) |
| | | \|\|\|\|\|\|\|\|\|\|\|\|\|\|\|\| \|\|\| \|\|\|\|\|\|\|+ \|+\|\|\| \|\|\|\|\|\|\|\|\|\|\|\|\|\|\|\|\|\|\|\|\|\| | | |
| Sbjct: | 68 | MFQFHAGSWESWCCCCCLIPGDRPWDRGRRWRLEMRNTRSVHETRFEAAVKVIQSLPKNG | 247 | (SEQ ID NO:89) |
| Query: | 60 | SFQPTNEMMLKFYSFYKQATEGPCKLSRPCFWDPIGRYKWDAWSSLGDMTKEEANIAYVE | 119 | |
| | | \|\|\|\|\|\|\|\|\|\|\|\|\|\|\|\|\|\|\|\|\|\|\|\|\|\|\|+\|\|\|\|\|\|+\|\|\|\|\|\|\|\|\|\|\|\|\|\|\|\|\|\|\|\|\|\|\|\|\| | | |
| Sbjct: | 248 | SFQPTNEMMLKFYSFYKQATEGPCKLSKPGFWDPVGRYKWDAWSSLGDMTKEEAMIAYVE | 427 | |
| Query: | 120 | EMKKIIETMPMTEKVEELLRVIGPFYEIVEDKKSGRSSDITSVRLEKISKCLEDLGNVLT | 179 | |
| | | \|\|\|\|\|+\|\|\|\|\|\|\|\|\|\|\|\|\| \|\|\|\|\|\|\|\|\|\|\|\|\|\|\|\|\|\|\|+\|\|\|\|\|\|\|\|\|\|\|\|\|\|\|\|\|\|\| | | |
| Sbjct: | 428 | EMKKILETMPMTEKVEELLHVIGPFYEIVEDKKSGRSSDLTSVRLEKISKCLEDLGNVLA | 607 | |
| Query: | 180 | STPNAKTVNGKAESSDSGAESEEEEAQEEVKGAEHSDNDKKNMKKSADHKNLEVIVTNGY | 239 | |
| | | \|\|\|\|\|\|\|\|\|\|\|\|\|\|\|\|\|\|\|\|\|\|\|\| \|\|\|+ \| \| \|+\|\|\|\|\|\|\|\|\|\|\|\|\|\|\|+\|\|\|\|\|\| | | |
| Sbjct: | 608 | STPNAKTVNGKAESSDSGAESEEEAAQEDPKRPEPRDSDKKMMKKSADHKNLEIIVTNGY | 787 | |
| Query: | 240 | DKDGFVQDIQNDIHASSSLNGRSTEEVKPIDENLGQTGKSAVCIHQGINDDHVEDVTGIQ | 299 | |
| | | \|\|\| \|\|\| +\|\| \|\| \| \|\|\|\|\| \|\|\|\|\| +\|\|\|\| \|\|\|\|+ \| +\|\| +\| \|\|\|\|\|++\|\|\| | | |
| Sbjct: | 788 | DKDSFVQGVQNSIHTSPSLNGRCTEEVKSVDENLEQTGKTVVFVHQDVNSDHVEDISGIQ | 967 | |
| Query: | 300 | HLTSDSDSEVYCDSMEQFGQEESLDSFTSNNGPFQYYLGGHSSQPMENSGFREDIQVPPG | 359 | |
| | | \|\|\|\|\|\|\|\|\|\|\|\|\|\|\|\|\|\|\|\|\|\|\|\|\| \| \|\|\|\|\|\| \|\|\|\|\|+ \|\|\|+\|+\|\|\| \| +\| \|\| | | |
| Sbjct: | 968 | HLTSDSDSEVYCDSMEQFGQEESLDGFISNNGPFSYYLGGNPSQPLESSGFPEAVQGLPG | 1147 | |
| Query: | 360 | NGNIGNMQVVAVEGKGEVKHGGEDGRNNSGAPHREKRGGETDEFSNVRRGRGHRMQHLSE | 419 | |
| | | \|\|+ +\|\| \|\|\|\|\|\|\|\| \|\|\|\|\| +\|\|\|\|\|\|\|\|\|\| \|\|++\|\|\|\|+\|\|\|\|\|\|\|\|\|\|\|\|\| | | |
| Sbjct: | 1148 | NGSPEDMQGAVVEGKGEVKRGGEDGGSNSGAPHREKRAGESEEFSNIRRGRGHRMQHLSE | 1327 | |
| Query: | 420 | GTKGRQVGSGGDGERWGSDRGSRGSLNEQIALVLMRLQEDMQNVLQRLQKLETLTAAKSS | 479 | |
| | | \|+\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\|\| \| \|+++ | | |
| Sbjct: | 1328 | GSKGRQVGSGGDGERWGSDRGSRGSLNEQIALVLMRLQEDMQNVLQRLHKLEMLAASQAK | 1507 | |
| Query: | 480 | TSTLQTAPQPTSSQRPSWWPFEMSPGVLTFAIIWPFIAQWLVYLYYQRRRR 530 | | |
| | | +\| \|\|\|+ \|\|\|\| \|\|\|\|\|\|\|\|\|\|\|\| \|\|\|\|\|\|\|\|\|\|\|\|\|\|\|+\|\|\|\|\|\|\|\| | | |
| Sbjct: | 1508 | SSALQTSNQPTSP-RPSWWPFEMSPGALTFAIIWPFIAQWLVHLYYQRRRR 1657 | | |

An ENDO6 nucleic acid of the invention can include a nucleic acid encoding the polypeptide of SEQ ID NO:23, e.g., the ENDO6 nucleic acid can include the nucleic acid sequence of SEQ ID NO:22. The invention also includes a mutant or variant nucleic acid any of whose bases may be changed from the corresponding base shown in Table 27A. In some embodiments, the ENDO6 nucleic acid encodes a protein that maintains its endozepine-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, but are not limited to: modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

An ENDO6 polypeptide of the invention can include the amino acid sequence of SEQ ID NO:23. The invention also includes a mutant or variant protein any of whose residues may be changed from the corresponding residue shown in SEQ ID NO:23, while still encoding a protein that maintains its endozepine-like activities and physiological functions, or a functional fragment thereof, such as the active peptide (SEQ ID NO:24)

Metabolism-Regulating Peptide #8 (MRP-8) Sequence:

QATEGPCKLSRPGFWDP (SEQ ID NO:24)

The invention further encompasses antibodies and antibody fragments, such as F_{ab} or (F_{ab})₂, that bind immunospecifically to the ENDO6 polypeptide, and derivatives and fragments, thereof.

An ENDO6 sequence is useful for detecting specific types of tissue. For example when a panel of tissue is assayed for expression, ENDO6 is highly expressed in skeletal muscle. Also, high expression of ENDO6 is a marker for multiple types of cancer.

An ENDO6 sequence is also useful to modulate global energy metabolism or weight by altering serum cholesterol and insulin.

An ENDO6 sequence is also useful in a method to identity the cellular receptors and downstream effectors of the invention by any one of a number of techniques commonly employed in the art.

An ENDO6 sequence is useful in the treatment of diabetes, metabolic disturbances associated with obesity, the metabolic syndrome X as well as anorexia and wasting disorders associated with chronic diseases and various cancers by modulating metabolism.

ENDO7

An ENDO7 nucleic acid of the invention includes the nucleic acid sequence shown in Table 27D (SEQ ID NO:25).

TABLE 27D

| | | |
|---|---|---|
| 1 | CCAGTATGTCTCAGGCGTTTGAGAAAGCTGCCAAGGATATTAAGCACCTTGAGACCAAGCCAGCAGATGATGAGAGGATG | (SEQ ID NO:25) |
| 81 | TTCATCTACAGCCGCTGCAAACAAGCGACTGTGCATGACTTAAATACAGAATGGCCCAGGATGTTAGACCTCAAAGGCAA | |
| 161 | GGCAAAGCAGGATGCTTGGAATGAGCTGAAAGACACTGCCAAGGAAGATGCTGTGAAAGCTGATATCAACAAAGTAGAAG | |
| 241 | AGCGAAATAAAAAATACAGAATATAAGAGATTG | |

The nucleic acid sequence disclosed in Table 27D includes an open reading frame (“ORF”) beginning at position 6 with start and stop codons indicated in bold. The ORF encodes a polypeptide sequence of 86 amino acid residues. The sequence of this encoded polypeptide is presented in Table 27E (SEQ ID NO:26). The homology between the translated protein and Bovine endozepine (putative ligand of benzodiazepine receptor) (gb:GENBANK-ID:BOVEDZ|acc:M15886) is presented in Table 27F.

TABLE 27E

| | | |
|---|---|---|
| 1 | MSQAFEKAAKDIKHLETKPADDERMFIYSRCKQATVHDLNTEWPRMLDLKGKAKQDAWNELKDTAKEDAVKADINKVEER | (SEQ ID NO:26) |
| 81 | NKKYRI | |

TABLE 27F

>gb:GENBANK-ID:BOVEDZ|acc:M15886 Bovine endozepine (putative ligand of
benzodiazepine receptor) mRNA, complete cds—Bos taurus, 608 bp.
Length = 608
Plus Strand HSPs:
Score = 307 (108.1 bits), Expect = 6.5e−26, P = 6.5e−26
Identities = 62/87 (71%), Positives = 73/87 (83%), Frame = +2

| | | | | |
|---|---|---|---|---|
| Query: | 1 | MSQA-FEKAAKDIKHLETKPADDERMFIYSRCKQATVHDLNTEWPRMLDLKGKAKQDAWN | 59 | (SEQ ID NO:90) |
| | | \|\|\|\| \|+\|\|\|+++=51 \|\|+\|\|\|\|\|+\| +\|\|\|\| \|\|\|\|\| \|+\|\|\| \| \|\|\| \|\|\|\|\| \|\|\|\| | | |
| Sbjct: | 125 | MSQAEFDKAAEEVKHLKTKPADEEMLFIYSHYKQATVGDINTERPGMLDFKGKAKWDAWN | 304 | (SEQ ID NO:91) |
| Query: | 60 | ELKDTAKEDAVKADINKVEERNKKYRI 86 | | |
| | | \|\|\| \|+\|\|\|\|+\|\| \|+\|\|\|\| \|\|\| \| | | |
| Sbjct: | 305 | ELKGTSKEDAMKAYIDKVEELKKKYCI 385 | | |

An ENDO7 nucleic acid of the invention can include a nucleic acid encoding the polypeptide of SEQ ID NO:26, e.g., the ENDO7 nucleic acid can include the nucleic acid sequence of SEQ ID NO:25. The invention also includes a mutant or variant nucleic acid any of whose bases may be changed from the corresponding base shown in Table 27D. In some embodiments, the ENDO7 nucleic acid encodes a protein that maintains its endozepine-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, but are not limited to: modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

An ENDO7 polypeptide of the invention can include the amino acid sequence of SEQ ID NO:26. The invention also includes a mutant or variant protein any of whose residues may be changed from the corresponding residue shown in SEQ ID NO:26, while still encoding a protein that maintains its endozepine-like activities and physiological functions, or a functional fragment thereof such as the following active peptide (SEQ ID NO:27)

Metabolism-Regulating Peptide #9 (MRP-9) Sequence:

QATVHDLNTEWPRMLDLKGK (SEQ ID NO:27)

The invention further encompasses antibodies and antibody fragments, such as F_{ab} or (F_{ab})₂, that bind immunospecifically to the ENDO7 polypeptide, and derivatives and fragments, thereof.

An ENDO7 sequence is useful for detecting specific types of tissue. For example when a panel of tissue is assayed for expression, ENDO7 is highly expressed in adipose tissue. Also, high expression of ENDO7 is a marker for liver cancer.

An ENDO7 sequence is also useful to modulate global energy metabolism or weight by altering serum cholesterol.

An ENDO7 sequence is also useful in a method to identity the cellular receptors and downstream effectors of the invention by any one of a number of techniques commonly employed in the art.

An ENDO7 sequence is useful in the treatment of diabetes, metabolic disturbances associated with obesity, the metabolic syndrome X as well as anorexia and wasting disorders associated with chronic diseases and various cancers by modulating metabolism.

ENDO7A

ENDO7A is a variant of ENDO7. An ENDO7A nucleic acid of 428 nucleotides (also referred to as CG55229-O₂) (SEQ ID NO:182) encoding a novel ACYL-COENZYME A BINDING PROTEIN (DIAZEPAM-BINDING INHIBITOR)-like protein is shown in Table 38. An open reading frame was identified beginning at nucleotides 106-108 and ending at nucleotides 364-366. The start (ATG) and stop (TAA) codons of the open reading frame are highlighted in bold type. Putative untranslated regions (underlined), if any, are found upstream from the initiation codon and downstream from the termination codon.

TABLE 38

| | | |
|---|---|---|
| TTAATATTGCTATATTAGTTTTGCAATTGAAAAATTAAGTTCCAATAGCTTCCTGCTCTG | 60 | (SEQ ID NO:182) |
| TACCTTCTCAGTGGGCTCCCAGGAATCCTTGCAACAACTGCCAGTATGTCTCAGGCGTTT | 120 | |
| GAGAAAGCTGCCAAGGATATTAAGCACCTTGAGACCAAGCCAGCAGATGATGAGAGGATG | 180 | |
| TTCATCTACAGCCGCTGCAAACAAGCGACTGTGCATGACTTAAATACAGAATGGCCCAGG | 240 | |
| ATGTTAGACCTCAAAGGCAAGGCAAAGCAGGATGCTGGNAATGAGCTGAAAGACACTGCC | 300 | |
| AAGGAAGATGCTGTGAAAGCTGATATCAACAAAGTAGAAGAGCGAAATAAAAAATACAGA | 360 | |
| ATATAAGAGATTGGATTTGGTTGCCAGCANTGCATTTAACCTAAACTGATACAATGCCTT | 420 | |
| TTTTTCCC | | |

In a search of sequence databases, it was found, for example, that the nucleic acid sequence of ENDO7A has 304 of 381 bases (79%) identical to a gb:GENBANK-ID:HUMEDZ|acc:M15887.1 mRNA from Homo sapiens (Human endozepine (putative ligand of benzodiazepine receptor) mRNA, complete cds).

The ENDO7A encoded protein having 86 amino acid residues (SEQ ID NO:183) is presented using the one-letter code in Table 39. Although the PSORT, SignalP and hydropathy profile results predict that this sequence has no signal peptide and is likely to be localized in the nucleus with a certainty of 0.5833 predicted by PSORT, proteins of this family are generally secreted and hence we predict that protein of invention is also secreted. Alternatively, ENDO7A is likely to be localized to the mitochondrial matrix space with a certainty of 0.1000 or to the lysosome lumen with a certainty of 0.1000.

TABLE 39

| | | |
|---|---|---|
| MSQAFEKAAKDIKHLETKPADDERMFIYSRCKQATVHDLNTEWPRMLDLKGKAKQDAGNE | 60 | (SEQ ID NO:183) |
| LKDTAKEDAVKADINKVEERNKKYRI | | |

The full amino acid sequence of ENDO7A was found to have 61 of 87 amino acid residues (70%) identical to, and 72 of 87 amino acid residues (82%) similar to, the 87 amino acid residue ptnr:REMTREMBL-ACC:CAA44618 protein from synthetic construct (ACYL-COA-BINDING PROTEIN/DIAZEPAM-BINDING INHIBITOR).

In a further search of public sequence databases, ENDO7A was found to have homology to the amino acid sequences shown in the BLASTP data listed in Table 40.

TABLE 40
BLASTP results for ENDO7A

| Gene Index/ | | Length | Identity | Positives | |
|---|---|---|---|---|---|
| Identifier | Protein/Organism | (aa) | (%) | (%) | Expect |
| REMTREMBL- | ACYL-COA-BINDING | 87 | 61/87 | 72/87 | 1.1e−25 |
| ACC: CAA44618 | PROTEIN /DIAZEPAM- | | (70%) | (82%) | |
| | BINDING INHIBITOR - | | | | |
| | synthetic construct | | | | |
| SPTREMBL- | ENDOZEPINE - | 87 | 62/87 | 72/87 | 1.8e−25 |
| ACC: Q9TSG2 | Sus scrofa | | (71%) | (82%) | |
| SPTREMBL- | ACYL-COENZYME A | 86 | 58/86 | 72/86 | 3.0e−25 |
| ACC: Q9TQX6 | BINDING PROTEIN, ACBP - | | (67%) | (83%) | |
| | Canis familiaris | | | | |
| SWISSPROT- | Acyl-CoA-binding | 86 | 60/86 | 71/86 | 3.8e−25 |
| ACC: P07107 | protein (ACBP) | | (69%) | (82%) | |
| | (Diazepam binding | | | | |
| | inhibitor) (DBI) | | | | |
| | (Endozepine) (EP) - | | | | |
| | Bos taurus | | | | |
| SWISSPROT- | Acyl-CoA-binding | 86 | 61/86 | 71/86 | 6.3e−25 |
| ACC: P12026 | protein (ACBP) | | (70%) | (82%) | |
| | (Diazepam binding | | | | |
| | inhibitor) (DBI) | | | | |
| | (Endozepine) (EP) | | | | |
| | [Contains: DBI(32-86)] - | | | | |
| | Sus scrofa | | | | |

A multiple sequence alignment is given in Table 41 in a ClustalW analysis comparing ENDO1A with related protein sequences shown in Table 40.

[image]

BLAST analysis was performed on sequences from the Patp database, which is a proprietary database that contains sequences published in patents and patent publications. Patp results include those listed in Table 42.

TABLE 42
Patp BLASTP Analysis for ENDO7A

| Sequences | | | | | |
|---|---|---|---|---|---|
| producing High- | | | | | |
| scoring Segment | | Length | Identity | Positive | |
| Pairs | Protein/Organism | (aa) | (%) | (%) | E Value |
| AAB81818 | Human endozepine-like | 83 | 82/86 | 82/86 | 3.8e−38 |
| | ENDO7 SEQ ID NO: 26 - | | (95%) | (95%) | |
| | Homo sapiens | | | | |
| AAP60957 | Sequence of bovine | 111 | 61/87 | 72/87 | 9.4e−26 |
| | endogenous | | (70%) | (82%) | |
| | benzodiazepineoid | | | | |
| | (EBZD) polypeptide - Bos taurus | | | | |
| AAP60954 | Sequence of bovine | 86 | 60/86 | 71/86 | 3.2e−25 |
| | endogenous | | (69%) | (82%) | |
| | benzodiazepineoid | | | | |
| | (EBZD) polypeptide - Bos taurus | | | | |
| AAR11874 | Recombinant bovine | 86 | 60/86 | 71/86 | 3.2e−25 |
| | EBZD - Bos taurus | | (69%) | (82%) | |
| AAP60958 | Sequence of human | 107 | 59/87 | 70/87 | 6.6e−25 |
| | endogenous | | (67%) | (80%) | |
| | benzodiazepineoid | | | | |
| | (EBZD) polypeptide - | | | | |
| | Homo sapiens | | | | |

The presence of identifiable domains in the ENDO7A protein disclosed herein was determined by searches versus domain databases such as Pfam, PROSITE, ProDom, Blocks or Prints and then identified by the Interpro domain accession number. Significant domains are summarized in Table 43.

TABLE 43

| Model | Domain | seq-f | seq-t | hmm-f | hmm-t | score | E-value |
|---|---|---|---|---|---|---|---|
| -------- | ------- | ----- | ----- | ----- | ----- | ----- | ------- |
| ACBP | 1/1 | 1 | 85 | 1 | 89 | 78.6 | 1.3e−19 |

Alignments of top-scoring domains:
ACBP: domain 1 of 1, from 1 to 85: score 78.6, E = 1.3e−19

| | | | | |
|---|---|---|---|---|
| | | lqedFeaAaekvkkLkknGpvkPSneekLkLYsLYKQATvGDvnter | | (SEQ ID NO:189) |
| | | +++ \|\|+\|\|+ +\|+\| ++ \| ++\| ++\|\| \|\|\|\|\| \| \|\|\| | | |
| ENDO7A | 1 | MSQAFEKAAKDIKHLETK----PADDERMFIYSRCKQATVHDLNTEW | 43 | (SEQ ID NO:190) |
| | | PGmfDlkgrAKWDAWnelkGmSkeeAmkaYIakVeeLiakya | | |
| | | \| \|+\|\|\|\|+\|\| \|\| \|\|\|\|+++\|\|+\| \|\| \| \|\|\|\| +\|\| | | |
| ENDO7A | 44 | PRMLDLKGKAKQDAGNELKDTAKEDAVKADINKVEERNKKYR 85 | | |

Acyl-CoA-binding protein (ACBP) is a small (10 Kd) protein that binds medium- and long-chain acyl-CoA esters with high affinity, and may act as an intra-cellular carrier of acyl-CoA esters. ACBP has a number of important physiological and biochemical functions: it is known as a diazepam binding inhibitor, as a putative neurotransmitter, as a regulator of insulin release from pancreatic cells, and as a mediator in corticotropin-dependent adrenal steroidogenesis. It is possible that the protein acts as a neuropeptide that takes part in the modulation of gamma-aminobutyric acid-ergic transmission. The structure of ACBP has been deduced by NMR spectroscopy and has been shown to be a mainly-alpha protein, consisting of 5 short alpha-helices and 3 connecting beta-strands (Marquardt H et al., 1986, J. Biol. Chem. 261: 9727-9731; Poulsen F. M., Andersen K. V., 1992, J. Mol. Biol. 226: 1131-1141). ACBP is a highly conserved protein of about 90 residues that has been so far found in vertebrates, insects, plants and yeast. Other proteins belonging to the ACBP family include mouse endozepine-like peptide (ELP) (gene DBIL5); mammalian MA-DBI, a transmembrane protein of unknown function which has been found in mammals; and human DRS-1, a protein of unknown function that contains a N-terminal ACBP-like domain and a C-terminal enoyl-CoA isomerase/hydratase domain (Ivell R. et al., 1996, Mol. Cell. Endocrinol. 122: 69-80; Suk K et al., 1999, Biochim. Biophys. Acta 1454:126-131).

This indicates that the sequence of the invention has properties similar to those of other proteins known to contain this/these domain(s) and similar to the properties of these domains.

The ACYL-COENZYME A BINDING PROTEIN (DIAZEPAM-BINDING INHIBITOR)-like ENDO7A disclosed in this invention is expressed in at least the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus.

The nucleic acids and proteins of the invention have applications in the diagnosis and/or treatment of various diseases and disorders. For example, the compositions of the present invention will have efficacy for the treatment of patients suffering from: cancer, trauma, immunological disease, respiratory disease, gastro-intestinal diseases, reproductive health, neurological and neurodegenerative diseases, bone marrow transplantation, metabolic and endocrine diseases, allergy and inflammation, nephrological disorders, hematopoietic disorders or urinary system disorders as well as other diseases, disorders and conditions.

These materials are further useful in the generation of antibodies that bind immunospecifically to the novel substances of the invention for use in diagnostic and/or therapeutic methods. ENDO8

An ENDO8 nucleic acid of the invention includes the nucleic acid sequence shown in Table 27G (SEQ ID NO:28).

TABLE 27G

(SEQ ID NO:28)

| | |
|---|---|
| 1 | ATGTGGGGCGACCTCTGGCTCCTCCCGCCTGCCTCTGCCAATCCGGGCACTGGGACAGAGGCTGAGTTTGAGAAAAGCTGC |
| 81 | AGAGGAGGTTAGGCACCTTAAGACCAAGCCATCGGATGAGGAGATGCTGTTCATCTATGGCCACTACAAACAAGCAACTG |
| 161 | TGGGCGACATAAATACAGAACGGCCCGGGATGTTGGACTTCACGGGCAAGGCCAAGTGGGATGCCTGGAATGAGCTGAAA |
| 241 | GGGACTTCCAAGGAAGATGCCATGAAAGCTTACATCAACAAAGTAGAAGAGCTAAAGAAAAAATACGGGATATGA |

The nucleic acid sequence disclosed in Table 27G includes an open reading frame (“ORF”) beginning at position 1 with start and stop codons in bold. The ORF encodes a polypeptide sequence of 104 amino acid residues. The sequence of this encoded polypeptide is presented in Table 27H (SEQ ID NO:29). The homology between the translated protein and Human diazepam binding inhibitor (DBI) (gb:GENBANK-ID:HUMDBI|acc:M14200) is presented in Table 27I.

TABLE 27H

(SEQ ID NO:29)

| | |
|---|---|
| 1 | MWGDLWLLPPASANPGTGTEAEFEKEEVRHLKTKPSDEEMLFIYGHYKQATVGDINTERPGMLDFTGKAKWDAWNELK |
| 81 | GTSKEDAMKAYINKVEELKKKYGI |

An ENDO8 nucleic acid of the invention can include a nucleic acid encoding the polypeptide of SEQ ID NO:29, e.g., the ENDO8 nucleic acid can include the nucleic acid sequence of SEQ ID NO:28. The invention also includes a mutant or variant nucleic acid any of whose bases may be changed from the corresponding base shown in Table 27G. In some embodiments, the ENDO8 nucleic acid encodes a protein that maintains its endozepine-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, but are not limited to: modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

An ENDO8 polypeptide of the invention can include the amino acid sequence of SEQ ID NO:29. The invention also includes a mutant or variant protein any of whose residues may be changed from the corresponding residue shown in SEQ ID NO:29, while still encoding a protein that maintains its endozepine-like activities and physiological functions, or a functional fragment thereof such as the following active peptide (SEQ ID NO:30)

Metabolism-Regulating Peptide #1 (MRP-1) Sequence:

1 QATVGDINTERPGMLDFTGK (SEQ ID NO:30)

The invention further encompasses antibodies and antibody fragments, such as F_{ab} or (F_{ab})₂, that bind immunospecifically to the ENDO8 polypeptide, and derivatives and fragments, thereof.

An ENDO8 sequence is useful for detecting specific types of tissue. For example when a panel of tissue is assayed for expression, ENDO8 is highly expressed in heart skeletal muscle, liver and endothelial tissue. Also, high expression of ENDO8 is a marker for breast and colon cancers as well as melanoma.

An ENDO8 sequence is also useful to modulate global energy metabolism or weight by altering serum cholesterol, insulin, or glucose. The ENDO8 sequence is also useful to modulate muscle mass or adipose level.

An ENDO8 sequence is also useful in a method to identity the cellular receptors and downstream effectors of the invention by any one of a number of techniques commonly employed in the art.

An ENDO8 sequence is useful in the treatment of diabetes, metabolic disturbances associated with obesity, the metabolic syndrome X as well as anorexia and wasting disorders associated with chronic diseases and various cancers by modulating metabolism.

ENDO9

An ENDO9 nucleic acid of the invention includes the nucleic acid sequence shown in Table 27J (SEQ ID NO:31).

TABLE 27J

(SEQ ID NO:31)

| | |
|---|---|
| 1 | ATGAGAGCCAGTCAGAAGGACTTTGAAATTCAATGAATCAAGTGAAACTCTTGAAAAATCCAGGAAACGAAGTGAA |
| 81 | GCTAAACTCTACGCGCTATATAAGCAGGCCACTGAAGGACCTTGTAACATGCCCAAACCAGGTGTATTTGACTTGATCA |
| 161 | ACAAGGCCAATGGGACGCATGGAATGCCCTTGGCAGCCTGCCCAAGGAAGCTGCCAGGCAGAACTATGTGGATTTGGTG |
| 241 | TCCAGTTTGAGTCCTTCATTGGATCCTCTAGTCAGGTGGAGCCTGGAACAGACAGGAAATCAACTGGGTTTGAAACTCT |
| 321 | GGTGGTGACCTCCGAAGATGGCATCACAAAGATCATGTTCAACCGGCCCAAAAAGAAAAATGCCATAAACACTGAGATGT |
| 401 | ATCATGAAATTATGCGTGCACTTAAAGCTGCCAGCAAGGATGACTCAATCATCACTGTTTTAACAGGAAATGGTGACTAT |
| 481 | TACAGTAGTGGGAATGATCTGACTAACTTCACTGATATTCCCCCTGGTGGAGTAGAGGAGAAAGCTAAAAATAATGCCGT |
| 561 | TTTACTGAGGGATTTGTGGGCTGTTTTATAGATTTTCCTAAGCCTCTGATTGCAGTGGTCAATGGTCCAGCTGTGGGCA |
| 641 | TCTCCGTCACCCTCCTTGCTATTCGATGCCGTGTATGCATCTGACAGGGCAACATTTCATACACCATTTAGTCACCTA |
| 721 | GGCCAAGTCCGGAAGGATGCTCCTCTTACACTTTTCCGAAGATAATGAGCCCAGCCAAGGCAACAGAGATGCTTATTTT |
| 801 | TGGAAAGAAGTTAACAGCGGGAGAGGCATGTGCTCAAGGACTTGTTACTGAAGTTTTCCCTGATAGCACTTTTCAGAAAG |
| 881 | AAGTCTGGACCAGGCTGAAGGCATTTGCAAAGCTTCCCCCAAATGCCTTGAGAATTTCAAAAGAGGTAATCAGGAAAAGA |
| 961 | GAGAGAGAAAAACTACACGCTGTTAATGCTGAAGAATGCAATGTCCTTCAGGGAAGATGGCTATCAGATGAATGCACAAA |
| 1041 | TGCTGTGGTGAACTTCTTATCCAGAAAATCAAAACTGTGA |

The nucleic acid sequence disclosed in Table 27J includes an open reading frame (“ORF”) beginning at position 1 with start and stop codons in bold. The ORF encodes a polypeptide sequence of 359 amino acid residues. The sequence of this encoded polypeptide is presented in Table 27K (SEQ ID NO:32). The homology between the translated protein and Homo sapiens peroxisomal D3,D2-enoyl-CoA isomerase (PECI) (gb:GENBANK-ID:AF153612|acc:AF153612) is presented in Table 27L.

TABLE 27K

(SEQ ID NO:32)

| | |
|---|---|
| 1 | MRASQKDFENSMNQVKLLKKDPGNEVKLKLYALYKQATEGPCNMPKPGVFDLINKAKWDAWNALGSLPKEAARQNYVDLV |
| 31 | SSLSPSLESSSQVEPGTDRKSTGFETLVVTSEDGITKIMFNRPKKKNAINTEMYHEIMRALKAASKDDSIITVLTGNGDY |
| 161 | |
| 241 | GQSPEGCSSYTFPKIMSPAKATEMLIFGKKLTAGEACAQGLVTEVFPDSTFQKEVWTRLKAFAKLPPNALRISKEVIRKR |
| 321 | EREKLHAVNAEECNVLQGRWLSDECTNAVVNFLSRKSKL |

An ENDO9 nucleic acid of the invention can include a nucleic acid encoding the polypeptide of SEQ ID NO:32, e.g., the ENDO9 nucleic acid can include the nucleic acid sequence of SEQ ID NO:31. The invention also includes a mutant or variant nucleic acid any of whose bases may be changed from the corresponding base shown in Table 27J. In some embodiments, the ENDO9 nucleic acid encodes a protein that maintains its endozepine-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, but are not limited to: modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

An ENDO9 polypeptide of the invention can include the amino acid sequence of SEQ ID NO:32. The invention also includes a mutant or variant protein any of whose residues may be changed from the corresponding residue shown in SEQ ID NO:32, while still encoding a protein that maintains its endozepine-like activities and physiological functions, or a functional fragment thereof such as the following active peptide (SEQ ID NO:33)

Metabolism-Regulating Peptide #2 (MRP-2) Sequence:

QATEGPCNMPKPGVFDLINK (SEQ ID NO:33)

The invention further encompasses antibodies and antibody fragments, such as F_{ab} or (F_{ab})₂, that bind immunospecifically to the ENDO9 polypeptide, and derivatives and fragments, thereof.

An ENDO9 sequence is also useful to modulate global energy metabolism or weight by altering serum cholesterol, insulin, or glucose.

An ENDO9 sequence is also useful in a method to identity the cellular receptors and downstream effectors of the invention by any one of a number of techniques commonly employed in the art.

An ENDO9 sequence is useful in the treatment of diabetes, metabolic disturbances associated with obesity, the metabolic syndrome X as well as anorexia and wasting disorders associated with chronic diseases and various cancers by modulating metabolism.

ENDO10

An ENDO10 nucleic acid of the invention includes the nucleic acid sequence shown in Table 27M (SEQ ID NO:34).

TABLE 27M

(SEQ ID NO:34)

| | |
|---|---|
| 1 | TCCTTCCCCCACCCCCGGGGGCCCATCCCGGTGGCGGGCTCCGGAGCTCGGGACTGCTAATTTCAGCGAAACGATTAAAA |
| 81 | GACGCCCCTACAGCTGACGGCACTTTCTCTCCTCCGGCAGGANAGGACGTCCAGCGTACGTCNGCCCGCGCTTCCCCGCC |
| 161 | GGCGCAGAGCAGGCCTCACAGAATCGCACGCCGCTGGCACGCACGCCGCCCCGCCCCCACGGCCCAGCGCCAGCGCGCCC |
| 241 | CGCGTCGCACGCATCCCGGCCTCACTGCCCCTCGACTCCTGTTCCGTTGGAGGGGCCTGAGGCGAGCCTGAGCGCGCTGT |
| 321 | TGGCCGGAGAGCCGGAGAGACCGGGTCGACTGGGCAGAGCGGCAGAGGGTCGAGGAGCCTGCTCTGCACGCCCAGGGA |
| 401 | GTAGAAGTGGGCAGGGAGCAGGGTCACGTGAGGGAACGCGCCGCGACTGAGCTTGGGTCCGACTGGAGCTCAGGCTCGCG |
| 481 | ACCCAGACTGGTGGGCCAAGGCCTCCAAGCCGGCCTTACACCCAATCCAAGGAGGACAGACCGGAGGACACAGGGACGGAGC |
| 561 | GAGCAAGGAGACATGGCTTCATCATTCCTGCCCGCGGGGGCCATCACCGGCGACAGCGGTGGAGAGCTGAGCTCAGGGGA |
| 641 | CGACTCCGGGGAGGTGGAGTTCCCCCATAGCCCTGAGATCGAGGAGACCAGTTGCCTGGCCGAGCTGTTTGAGAAGGCTG |
| 721 | CCGCGCACCTGCAAGGCCTGATTCAGGTGGCCAGCAGGGAGCAGCTCTTGTACCTGTATGCCAGGTACAAACAGGTCAAA |
| 801 | GTTGGAAATTGTAATACTCCTAAACCAAGCTTCTTTGATTTTGAAGGAAAGCAAAAATGGGAAGCTTGGAAAGCACTTGG |
| 881 | TGATTCAAGCCCCAGCCAAGCAATGCAGGAATATATCGCAGTAGTTAAAAAACTAGATCCAGGTTGGAATCCTCAGATAC |
| 961 | CAGAGAAGAAAGGAAAAGAAGCAAATACAGGTTTTGGTGGGCCAGTTATTAGTTCTCTATATCATGAAGAAACCATCAGG |
| 1041 | GAAGAAGACAAAAATATATTTGATTACTGCAGGGAAAACAACATTGACCATATAACCAAAGCCATCAAATCGAAAAATGT |
| 1121 | GGATGTGAATGTGAAGATGAAGAGGGTAGGGCTCTACTTCACTGGGCCTGTGATCGAGGACATAAGGAACTAGTCACAG |
| 1201 | TGTTGCTGCAACATAGAGCTGACATTAACTGTCAGGACAATGAAGGCCAAACAGCTCTACATTATGCCTCTGCCTGTGAG |
| 1281 | TTTCTGGATATTGTAGAGCTGCTGCTCCAGTCTGGTGCTGACCCCACTCTCCGAGACCAGGATGGCTGCCTGCCAGAGGA |
| 1361 | GGTGACAGGCTGCAAAACAGTTTCTTTGGTGCTGCAGCGGCACACAACTGGCAAGGCTTAATCAAAAGACTGGAAAACTG |
| 1441 | CAGTCTGTAATAGCATAAGGCTTCCATTATGAAAGAAAACTACAAAAATAATACTTCTTTTCCACCCGTCTTTGGTATGT |
| 1521 | ATTGGCTAATAAAATCAGTTCTGTGGAACTGGGAAAAAAAAAAAAAAAAAAAAA |

The nucleic acid sequence disclosed in Table 27M includes an open reading frame (“ORF”) beginning at position 573 with start and stop codons in bold. The ORF encodes a polypeptide sequence of 282 amino acid residues. The sequence of this encoded polypeptide is presented in Table 27N (SEQ ID NO:35). The homology between the translated protein and (Human DBI/ACBP-like protein patent: U.S. Pat. No. 5,734,038-A 2 Mar. 31, 1998) is presented in Table 27O.

TABLE 27N

(SEQ ID NO:35)

| | |
|---|---|
| 1 | MASSFLPAGAITGDSGGELSSGDDSGEVEFPHSPEIEETSCLAELFEKAAAHLQGLIQVASREQLLYLYARYKQVKVGNC |
| 81 | NTPKPSFFDFEGKQKWEAWKALGDSSPSQAMQEYIAVVKKLDPGWNPQIPEKKGKEANTGFGGPVISSLYHEETIREEDK |
| 161 | NIFDYCRENNIDHITKAIKSKNVDVNVKDEEGRALLHWACDRGHKELVTVLLQHRADINCQDNEGQTALHYASACEFLDI |
| 241 | VELLLQSGADPTLRDQDGCLPEEVTGCKTVSLVLQRHTTGKA |

An ENDO10 nucleic acid of the invention can include a nucleic acid encoding the polypeptide of SEQ ID NO:35, e.g., the ENDO10 nucleic acid can include the nucleic acid sequence of SEQ ID NO:34. The invention also includes a mutant or variant nucleic acid any of whose bases may be changed from the corresponding base shown in Table 27M. In some embodiments, the ENDO10 nucleic acid encodes a protein that maintains its endozepine-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, but are not limited to: modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

An ENDO10 polypeptide of the invention can include the amino acid sequence of SEQ ID NO:35. The invention also includes a mutant or variant protein any of whose residues may be changed from the corresponding residue shown in SEQ ID NO:35, while still encoding a protein that maintains its endozepine-like activities and physiological functions, or a functional fragment thereof such as the following active peptide (SEQ ID NO:36)

Metabolism-Regulating Peptide #10 (MRP-10) Sequence:

1 QVKVGNCNTPKPSFFDFEGK (SEQ ID NO:36)

The invention further encompasses antibodies and antibody fragments, such as F_{ab} or (F_{ab})₂, that bind immunospecifically to the ENDO10 polypeptide, and derivatives and fragments, thereof.

An ENDO10 sequence is also useful to modulate global energy metabolism or weight by altering serum insulin or glucose. The ENDO10 sequence is also useful to modulate muscle mass or adipose level

An ENDO10 sequence is also useful in a method to identity the cellular receptors and downstream effectors of the invention by any one of a number of techniques commonly employed in the art.

An ENDO10 sequence is useful in the treatment of diabetes, metabolic disturbances associated with obesity, the metabolic syndrome X as well as anorexia and wasting disorders associated with chronic diseases and various cancers by modulating metabolism.

ENDO11

An ENDO11 nucleic acid of 1979 nucleotides (SEQ ID NO:191) encoding a novel Endozepine-like protein is shown in Table 44. An open reading frame was identified beginning with an ATG codon at nucleotides 459-461 and ending with a TGA codon at nucleotides 1374-1376. The start and stop codons of the open reading frame are highlighted in bold type. Putative untranslated regions (underlined), if any, are found upstream from the initiation codon and downstream from the termination codon.

TABLE 44

(SEQ ID NO:191)

| | |
|---|---|
| AGTAGGAAGCCGCGGGGTGGTGGCGAGAGAGGACCCAGGTGTCCTAGCAGTGGGCGCCGC | 60 |
| GGGGCACACGCTGGGCCAAGGTGCAGGCGGCCAGGGTGGGAGACTGTTCGCCCCGCCCTG | 120 |
| AGTACTCCTATCTTGTTTCTCCACCTGTTCGGGAGTTGGAGATGTGCACCTAAAGGAGGC | 180 |
| GCATCTGGGGACGGACACATCTGGCACTGAGGCCCTCGCCACCTGCCTCGCCACCTGGCG | 240 |
| ACCCTGACCCCACCACACTGCCTTGAGGTAGGAAAAGGAGGCTCCTCAACCACAACTTCT | 300 |
| GACCTCCCAGGGTGTCTGAGGCCTCTAAAGAGCTTAGTTTGCCCCTCTGGGAAGTGAATC | 360 |
| CTTGGCTTATGGTGCCGGGGGGACCCTGGAGGCCCCCTCACACGAAGGCTGCTTCTTGCA | 420 |
| GAGTCGCTCAAAAGTAGGGCCCCAGGGCTCGCAGCAGCATGGGCACCGAGAAAGAAAGCC | 480 |
| CAGAGCCCGACTGCCAGAAACAGTTCCAGGCTGCAGTGAGCGTCATCCAGAACCTGCCCA | 540 |
| AGAACGGTTCTTACCGCCCCTCCTATGAAGAGATGCTGCGATTCTACAGTTACTACAAGC | 600 |
| AGGCCACCATGGGGCCCTGCCTGGTCCCCCGGCCCGGGTTCTGGGACCCCATTGGACGAT | 660 |
| ATAAGTGGGACGCCTGGAACAGTCTGGGCAAGATGAGCAGGGAGGAGGCCATGTCTGCCT | 720 |
| ACATCACTGAAATGAAACTGGTGGCACAGAAGGTGATCGACACAGTGCCCCTGGGTGAGG | 780 |
| TGGCAGAGGACATGTTTGGTTACTTCGAGCCCCTGTACCAGGTGATCCCTGACATGCCGA | 840 |
| GGCCCCCAGAGACCTTCCTGAGAAGGGTCACAGGTTGGAAAGAGCAGGTTGTGAATGGAG | 900 |
| ATGTTGGGGCTGTTTCAGAGCCTCCCTGCCTCCCCAAGGAACCGGCACCCCCAAGCCCAG | 960 |
| AGTCCCATTCACCCAGGGACCTGGACTCCGAGGTTTTCTGTGATTCCCTGGAGCAGCTGG | 1020 |
| AGCCTGAGCTGGTTTGGACAGAGCAGCGGGCAGCATCTGGAGGAAAGCGTGATCCCAGGA | 1080 |
| ACAGCCCCGTGCCCCCCACAAAGAAAGAGGGGTTGCGGGGCAGCCCGCCGGGGCCCCAGG | 1140 |
| AGTTGGACGTGTGGCTGCTGGGGACAGTTCGAGCACTACAGGAGAGCATGCAGGAGGTGC | 1200 |
| AGGCGAGGGTGCAGAGCCTGGAGAGCATGCCCCGGCCCCCTGAGCAGAGGCCGCAGCCCA | 1260 |
| GGCCCAGTGCTCGGCCATGGCCCCTTGGGCTCCCGGGGCCCGCGCTGCTCTTCTTCCTCC | 1320 |
| TGTGGCCCTTCGTCGTCCAGTGGCTCTTCCGAATGTTTCGGACCCAAAAGAGGTGACTGT | 1380 |
| CAGTGGAGGGGTCTCTGCAGCCAACTGAGACTATCTTGCTGTGCCCTGAGCCTTCCTAGG | 1440 |
| GTTTAGAAGAACAGCATTCAAAATTCCCCGTCCTGTCAGTGTTTGCCTTCGCACCTCCTC | 1500 |
| CCCTAAAGCAGCGCGGGGGGCAAATAAGACCCCACCCCTCCCTGCAGCTTCACAGGGACG | 1560 |
| CTTCCTTCCCTCCCCGCAACCACCCCAGGCTCCCCTGGGAGGCTGCAGTTGTGGTACACG | 1620 |
| TCCCCGGTGCTGGGTTGGCCGTGACTCGGGGCCGGGGCGATCGGGTCTCAGCCCCTGCCT | 1680 |
| TCCCCAGTCTCTGGGTCACCCGAATTTTCCCACCCCTGCTTCTCCCCGAGGAGGTTGAGC | 1740 |
| TCTTGAGCAAGTTGGGACTTGGGCTGGGGCCTGGAAGAATGATTGGCTGGGAGGCCGCGG | 1800 |
| GAGGGAGGCCAGGAGGCCCGGACCAGTTGGGAGGAGTGAGCAGGCCCCGGGGGAGGGGGA | 1860 |
| TGAGCGCAGTTTGCTCGCTTTCCTCCCCTGCCGGCCCCCTCCGCCCCCACACACACTCGG | 1920 |
| GACGTCTTCATTGAAGATTCACTTACAAAGGAATGTTTCACTAAATAAAAGAAAACCAG | 1979 |

The ENDO11 disclosed in this invention maps to chromosome 17.

In a search of sequence databases, it was found, for example, that the nucleic acid sequence of ENDO11 has 1979 of 1979 bases (100%) identical to a gb:GENBANK-ID:AK023384|acc:AK023384.1 mRNA from Homo sapiens (Homo sapiens cDNA FLJ13322 fis, clone OVARC1001713, weakly similar to ENDOZEPINE-RELATED PROTEIN PRECURSOR).

An ENDO11 protein having 305 amino acid residues (SEQ ID NO:192) is presented using the one-letter code in Table 45. ENDO11 is likely to be a Type Ib (Nexo Ccyt) membrane protein with an integral likelihood of −2.66 (280-300). The PSORT, SignalP and hydropathy profile results predict that ENDO11 is likely to be localized at the plasma membrane with a certainty of 0.7000. In alternative embodiments ENDO11 is likely to be localized to the microbody (peroxisome) with a certainty of 0.2157, to the endoplasmic reticulum membrane with a certainty of 0.2000, or to the mitochondrial inner membrane with a certainty of 0.1000.

TABLE 45

| | | |
|---|---|---|
| MGTEKESPEPDCQKQFQAAVSVIQNLPKNGSYRPSYEEMLRFYSYYKQATMGPCLVPRPG | 60 | (SEQ ID NO:192) |
| FWDPIGRYKWDANNSLGKMSREEAMSAYITEMKLVAQKVIDTVPLGEVEADMFGYFEPLY | 120 | |
| QVIPDMPRPPETFLRRVTGWKEQVVNGDVGAVSEPPCLPKEPAPPSPESHSPRDLDSEVF | 180 | |
| CDSLEQLEPELVWTEQRAASGGKRDPRNSPVPPTKKEGLRGSPPGPQELDVWLLGTVRAL | 240 | |
| QESMQEVQARVQSLESMPRPPEQRPQPRPSARPWPLGLPGPALLFFLLWPFVVQWLFRMF | 300 | |
| RTQKR 305 | | |

The full amino acid sequence of the Endo-11 protein of the present invention was found to have 305 of 305 amino acid residues (100%) identical to the 305 amino acid residue ptnr:TREMBLNEW-ACC:BAB14553 protein from Homo sapiens (Human) (cDNA FLJ13322 FIS, CLONE OVARC1001713, WEAKLY SIMILAR TO ENDOZEPINE-RELATED PROTEIN PRECURSOR).

In a further search of public sequence databases, ENDO11 was found to have homology to the amino acid sequences shown in the BLASTP data listed in Table 46.

TABLE 46
BLASTP results for ENDO11

| Gene Index/ | | Length | Identity | Positives | |
|---|---|---|---|---|---|
| Identifier | Protein/Organism | (aa) | (%) | (%) | Expect |
| SPTREMBL- | OVARC1001713 PROTEIN - | 305 | 305/305 | 305/305 | 5.3e−170 |
| ACC: Q9H8Q4 | Homo sapiens | | (100%) | (100%) | |
| SWISSPROT- | Endozepine-related | 533 | 80/195 | 120/195 | 5.0e−44 |
| ACC: P07106 | protein precursor | | (41%) | (61%) | |
| | (Membrane-associated | | | | |
| | diazepam binding | | | | |
| | inhibitor) (MA-DBI) - Bos taurus | | | | |
| SWISSPROT- | Acyl-CoA-binding | 86 | 37/86 | 54/86 | 3.2e−13 |
| ACC: P12026 | protein (ACBP) | | (43%) | (62%) | |
| | (Diazepam binding | | | | |
| | inhibitor) (DBI) | | | | |
| | (Endozepine) (EP) | | | | |
| | [Contains: DBI(32-86)] - | | | | |
| | Sus scrofa | | | | |
| SPTREMBL- | ENDOZEPINE - Sus | 87 | 37/86 | 54/86 | 3.2e−13 |
| ACC: Q9TSG2 | scrofa | | (43%) | (62%) | |
| SWISSPROT- | Acyl-CoA-binding | 86 | 37/86 | 53/86 | 5.5e−13 |
| ACC: P07108 | protein (ACBP) | | (43%) | (61%) | |
| | (Diazepam binding | | | | |
| | inhibitor) (DBI) | | | | |
| | (Endozepine) (EP) - Homo sapiens | | | | |
| SPTREMBL- | HEPATOCELLULAR | 195 | 33/77 | 50/77 | 5.5e−13 |
| ACC: | CARCINOMA-ASSOCIATED | | (42%) | (64%) | |
| Q9NYD2 | ANTIGEN 64 - Homo sapiens | | | | |

A multiple sequence alignment is given in Table 47 in a ClustalW analysis comparing ENDO1A with related protein sequences shown in Table 46.

[image]

BLAST analysis was performed on sequences from the Patp database, which is a proprietary database that contains sequences published in patents and patent publications. Patp results include those listed in Table 48.

TABLE 48
Patp BLASTP Analysis for ENDO11

| Sequences | | | | | |
|---|---|---|---|---|---|
| producing High- | | | | | |
| scoring Segment | | Length | Identity | Positive | |
| Pairs | Protein/Organism | (aa) | (%) | (%) | E Value |
| AAB94579 | Human protein sequence | 305 | 305/305 | 305/305 | 4.4e−170 |
| | SEQ ID NO: 15373 - Homo sapiens | | (100%) | (100%) | |
| AAU27839 | Human full-length | 305 | 305/305 | 305/305 | 4.4e−170 |
| | polypeptide sequence | | (100%) | (100%) | |
| | #164 - Homo sapiens | | | | |
| AAM93535 | Human polypeptide, | 268 | 201/235 | 206/235 | 1.0e−106 |
| | SEQ ID NO: 3279 - Homo sapiens | | (85%) | (87%) | |
| AAG93321 | Human protein HP01124 - | 341 | 191/294 | 209/294 | 1.2e−94 |
| | Homo sapiens | | (64%) | (71%) | |
| AAB81816 | Human endozepine-like | 530 | 65/113 | 88/113 | 8.3e−51 |
| | ENDO6 SEQ ID NO: 23 - Homo sapiens | | (57%) | (77%) | |

The presence of identifiable domains in the ENDO11 protein disclosed herein was determined by searches versus domain databases such as Pfam, PROSITE, ProDom, Blocks or Prints and then identified by the Interpro domain accession number. Significant domains are summarized in Table 49.

TABLE 49

ACBP: domain 1 of 1, from 12 to 99: score 145.2, E = 1.1e-39

| | | | | |
|---|---|---|---|---|
| | | lqedFeaAaekvkkLkknGpvkPSneekLkLYsLYKQATvGDvnter | | (SEQ ID NO: 196) |
| | | \| +\| \|\|++++++\|+\|\|\|+ +\|\| \|\|+\| +\|\| \|\|\|\|\| \|++ +\| | | |
| ENDO11 | 12 | CQKQFQAAVSVIQNLPKNGSYRPSYEEMLRFYSYYKQATMGPCLVPR | 58 | (SEQ ID NO:197) |
| | | PGmfDlkgrAKWDAWnelkGmSkeeAmkaYIakVeeLiakya | | |
| | | \|\|++\|++\|\|+\|\|\|\|\|\|+\|+ \|\| \|\|\|\|+\|\|\|++++ \|+\|+ | | |
| ENDO11 | 59 | PGFWDPIGRYKWDAWNSLGKMSREEAMSAYITEMK-LVAQKV 99 | | |

This domain constitutes the hydrophobic binding site for acyl-CoA esters and is located within the second helical region of the molecule. The presence of a highly conserved gene in a primitive organism such as yeast supports its basic biologic role as an acyl-CoA-binding protein and suggests that many of the biologic functions attributed to it in higher organisms may result from its ability to interact with acyl-CoA.

This indicates that the sequence of the invention has properties similar to those of other proteins known to contain this/these domain(s) and similar to the properties of these domains.

The amino acid sequence of the protein of the invention has similarity to other endozepine-like proteins previously filed in a co-pending patent application by Curagen Corporation. These proteins were denoted as ENDO1, ENDO2, Endo3, ENDO4, Endo5, Endo6, ENDO7, Endo8, Endo9, and ENDO10. These endozepine-like proteins all contain a peptide subunit of 20 amino acids found within the ACBP domain and comprise what is the known human family of endozepine-like proteins.

An ENDO11 polypeptide of the invention can include the amino acid sequence of SEQ ID NO:192. The invention also includes a mutant or variant protein any of whose residues may be changed from the corresponding residue shown in SEQ ID NO:192, while still encoding a protein that maintains its endozepine-like activities and physiological functions, or a functional fragment thereof such as the following active peptide (SEQ ID NO:198).

Metabolism-Regulating Peptide #11 (MRP-11) Sequence:

QATMGPCLVPRPGFWDPIGR (SEQ ID NO:198).

The ENDO11 disclosed in this invention is expressed in at least the following tissues: uterus, cervix, foreskin, testes, and duodenal adenocarcenoma.

The nucleic acids and proteins of the invention have applications in the diagnosis and/or treatment of various diseases and disorders. For example, the compositions of the present invention will have efficacy for the treatment of patients suffering from: diabetes, obesity, metabolic syndrome X, as well as other diseases, disorders and conditions.

Animal studies have indicated that a 20 amino acid “peptide” found within each endozepine family member can biologically regulate metabolism. Such regulatory properties included the modulation of blood serum levels of glucose and cholesterol as well as the alteration of muscle mass and adipose depot. Specifically ENDO11 peptide is most homologous to ENDO6 peptide and therefore most likely to mimic its action biologically. Such action was found to be the lowering of serum levels of cholesterol as well as the lowering of serum levels of insulin.

ENDOX nucleic acids encode polypeptides that are novel members of the endozepine family. The endozepines have been reported to be involved in multiple biologically importatnt functions. Related endozepine polypeptides, such as diazepam binding inhibitor, bind GABA receptors. Accordingly, the new ENDOX polypeptides, or fragments or variants thereof, can be used in, e.g., screening assays to identify new agonists or antagonists for GABA receptors. The new ENDOX polypeptides and also be used to modulate GABA receptor activity.

ENDOX polypeptides, nucleic acids, antibodies, and other compositions according to the invention also have utilities based on other known functions of endozepine family members. For example, diazepam binding inhibitor, is also known as acyl-CoA binding protein (ACBP). Acyl-CoA-binding protein binds to medium- and long-chain acyl-CoA esters with high affinity, and may act as an intra-cellular carrier of acyl-CoA esters. Thus, The ACBP gene has also been cloned in yeast. The yeast cognate is named acyl-CoA binding (ACB) (Rose et al., Proc. Nat. Acad. Sci. (USA) 89: 11287-11291). The yeast gene encodes a polypeptide of 87 amino acid residues (including the initiating methionine), which is identical in length to the human gene product. The yeast polypeptide is 48% conserved with human amino acid residues. The most highly conserved yeast domain was found to comprise a total of 7 contiguous amino acid residues which are identical in all known protein species from yeast, birds, and mammals. This domain constitutes the hydrophobic binding site for acyl-CoA esters, and is located within the second helical region of the molecule. The presence of such a highly conserved gene in primitive organisms (e.g., yeast) supports its basic biological role as an acyl-CoA binding protein and also suggests that many of the biological functions attributed to it in higher organisms may result from its ability to interact with acyl-CoA.

Various endozepine family members, or derivatives of these polypeptides, have also been identified as antibacterial peptides. Examples include cecropin P1 and PR-39. PR-39 is a 39 amino acid residue proline- and arginine-rich polypeptide isolated from the upper part of pig small intestine. Amino acid sequence analysis in combination with mass spectrometry identified two of three of the the peptides as gastric inhibitory polypeptide (7-42) (GIP(7-42)) and diazepam-binding inhibitor (32-86) (DBI(32-86)), derived from factors which had been previously identified. The third polypeptide constituted a previously-unknown structure, which was designated peptide 3910, in relation to its molecular mass. All three polypeptides demonstrate antibacterial activity against Bacillus megaterium. GIP (7-42) also shows some activity against Streptococcus pyogenes and an Escherichia coli mutant with a defect in its outer membrane.

A summary of the ENDOX nucleic acid sequences, encoded ENDOX polypeptides, as well as Sequence Identifier Numbers (SEQ ID NOS) corresponding to various disclosed sequences and clones containing these nucleic acids, is shown in Table 28, below.

TABLE 28
Disclosed Sequences and Corresponding SEQ ID Numbers
of ENDOX Polypeptides

| | Sequence | ORF start and | Sequence | |
|---|---|---|---|---|
| | Identifier | stop codons | Identifier Number | Sequence |
| | Number of | from | of Encoded | Identifier |
| | Disclosed Nucleic | corresponding | Polypeptide | Number of Active |
| ENDOX | Acid Sequence | nucleic acid | Sequence | Peptide |
| ENDO1 | SEQ ID NO: 1 | 1-267 | SEQ ID NO: 2 | SEQ ID NO: 15 |
| | SEQ ID NO: 46 | 1-687 | SEQ ID NO: 47 | |
| | SEQ ID NO: 48 | 1-576 | SEQ ID NO: 49 | |
| ENDO1A | SEQ ID NO: 152 | 183-479 | SEQ ID NO: 153 | N/A |
| ENDO1B | SEQ ID NO: 156 | 1-684 | SEQ ID NO: 157 | N/A |
| ENDO2 | SEQ ID NO: 3 | 58-321 | SEQ ID NO: 4 | SEQ ID NO: 16 |
| ENDO2A | SEQ ID NO: 165 | 47-310 | SEQ ID NO: 166 | N/A |
| ENDO2B | SEQ ID NO: 174 | 398-661 | SEQ ID NO: 175 | N/A |
| ENDO3 | SEQ ID NO: 5 | 83-496 | SEQ ID NO: 6 | SEQ ID NO: 17, 18 |
| ENDO4 | SEQ ID NO: 7 | 11-298 | SEQ ID NO: 8 | SEQ ID NO: 19, 20 |
| ENDO4A | SEQ ID NO: 178 | 3-263 | SEQ ID NO: 179 | N/A |
| ENDO5 | SEQ ID NO: 9 | 7-264 | SEQ ID NO: 10 | SEQ ID NO: 21 |
| ENDO6 | SEQ ID NO: 22 | 1-1590 | SEQ ID NO: 23 | SEQ ID NO: 24 |
| ENDO7 | SEQ ID NO: 25 | 6-263 | SEQ ID NO: 26 | SEQ ID NO: 27 |
| ENDO7A | SEQ ID NO: 182 | 106-363 | SEQ ID NO: 183 | N/A |
| ENDO8 | SEQ ID NO: 28 | 1-312 | SEQ ID NO: 29 | SEQ ID NO: 30 |
| ENDO9 | SEQ ID NO: 31 | 1-1077 | SEQ ID NO: 32 | SEQ ID NO: 33 |
| ENDO10 | SEQ ID NO: 34 | 573-1418 | SEQ ID NO: 35 | SEQ ID NO: 36 |
| ENDO11 | SEQ ID NO: 191 | 459-1373 | SEQ ID NO: 192 | SEQ ID NO: 198 |

ENDOX Nucleic Acids and Polypeptides

One aspect of the invention pertains to isolated nucleic acid molecules that encode ENDOX polypeptides or biologically-active portions thereof. Also included in the invention are nucleic acid fragments sufficient for use as hybridization probes to identify ENDOX-encoding nucleic acids (e.g., ENDOX mRNAs) and fragments for use as PCR primers for the amplification and/or mutation of ENDOX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An ENDOX nucleic acid can encode a mature ENDOX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full length gene product, encoded by the corresponding gene. Alternatively, it may be defined as the polypeptide, precursor or proprotein encoded by an open reading frame described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an open reading frame, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated ENDOX nucleic acid molecules can contain less than about 5 kb, 4 kb, 3 kb, 2 kb, 1 kb,

0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence of SEQ ID NO: 1, 3, 5, 7, 9, 22, 25, 28, 31, 34, 46, 48, 152, 156, 165, 174, 178, 182, and 191, or a complement of this aforementioned nucleotide sequence, can be isolated using standard molecular biology techniques and the sequence information provided herein. Using all or a portion of the nucleic acid sequence of SEQ ID NO: 1, 3, 5, 7, 9, 22, 25, 28, 31, 34, 46, 48, 152, 156, 165, 174, 178, 182, and 191 as a hybridization probe, ENDOX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to ENDOX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NO: 1, 3, 5, 7, 9, 22, 25, 28, 31, 34, 46, 48, 152, 156, 165, 174, 178, 182, and 191, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NO: 1, 3, 5, 7, 9, 22, 25, 28, 31, 34, 46, 48, 152, 156, 165, 174, 178, 182, and 191, or a portion of this nucleotide sequence (e.g., a fragment that can be used as a probe or primer or a fragment encoding a biologically-active portion of an ENDOX polypeptide). A nucleic acid molecule that is complementary to the nucleotide sequence shown in SEQ ID NO: 1, 3, 5, 7, 9, 22, 25, 28, 31, 34, 46, 48, 152, 156, 165, 174, 178, 182, and 191, is one that is sufficiently complementary to the nucleotide sequence shown in SEQ ID NO: 1, 3, 5, 7, 9, 22, 25, 28, 31, 34, 46, 48, 152, 156, 165, 174, 178, 182, and 191, that it can hydrogen bond with little or no mismatches to the nucleotide sequence shown in SEQ ID NO: 1, 3, 5, 7, 9, 22, 25, 28, 31, 34, 46, 48, 152, 156, 165, 174, 178, 182, and 191, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of ENDOX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an ENDOX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human ENDOX protein. Homologous nucleic acid sequences include those nucleic acid sequences that encode conservative amino acid substitutions (see below) in SEQ ID NO:2, 4, 6, 8, 10, 15, 16, 17, 18, 19, 20, 21, 23, 24, 26, 27, 29, 30, 32, 33, 35 to 45 inclusive, 47, 49, 153, 157, 166, 175, 179, 183, 192, and 198, as well as a polypeptide possessing ENDOX biological activity. Various biological activities of the ENDOX proteins are described below.

An ENDOX polypeptide is encoded by the open reading frame (“ORF”) of an ENDOX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human ENDOX genes allows for the generation of probes and primers designed for use in identifying and/or cloning ENDOX homologues in other cell types, e.g. from other tissues, as well as ENDOX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NO: 1, 3, 5, 7, 9, 22, 25, 28, 31, 34, 46, 48, 152, 156, 165, 174, 178, 182, and 191; or an anti-sense strand nucleotide sequence of SEQ ID NO: 1, 3, 5, 7, 9, 22, 25, 28, 31, 34, 46, 48, 152, 156, 165, 174, 178, 182, and 191; or of a naturally occurring mutant of SEQ ID NO: 1, 3, 5, 7, 9, 22, 25, 28, 31, 34, 46, 48, 152, 156, 165, 174, 178, 182, and 191.

Probes based on the human ENDOX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an ENDOX protein, such as by measuring a level of an ENDOX-encoding nucleic acid in a sample of cells from a subject e.g., detecting ENDOX mRNA levels or determining whether a genomic ENDOX gene has been mutated or deleted.

“A polypeptide having a biologically-active portion of an ENDOX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of ENDOX” can be prepared by isolating a portion of SEQ ID NO: 1, 3, 5, 7, 9, 22, 25, 28, 31, 34, 46, 48, 152, 156, 165, 174, 178, 182, and 191, that encodes a polypeptide having an ENDOX biological activity (the biological activities of the ENDOX proteins are described below), expressing the encoded portion of ENDOX protein (e.g., by recombinant expression in vitro) and assessing the activity of the encoded portion of ENDOX.

ENDOX Nucleic Acid and Polypeptide Variants

The invention further encompasses nucleic acid molecules that differ from the nucleotide sequences shown in SEQ ID NO: 1, 3, 5, 7, 9, 22, 25, 28, 31, 34, 46, 48, 152, 156, 165, 174, 178, 182, and 191, due to degeneracy of the genetic code and thus encode the same ENDOX proteins as that encoded by the nucleotide sequences shown in SEQ ID NO: 1, 3, 5, 7, 9, 22, 25, 28, 31, 34, 46, 48, 152, 156, 165, 174, 178, 182, and 191. In another embodiment, an isolated nucleic acid molecule of the invention has a nucleotide sequence encoding a protein having an amino acid sequence shown in SEQ ID NO:2, 4, 6, 8, 10, 15, 16, 17, 18, 19, 20, 21, 23, 24, 26, 27, 29, 30, 32, 33, 35 to 45 inclusive, 47, 49, 153, 157, 166, 175, 179, 183, 192, and 198.

In addition to the human ENDOX nucleotide sequences shown in SEQ ID NO: 1, 3, 5, 7, 9, 22, 25, 28, 31, 34, 46, 48, 152, 156, 165, 174, 178, 182, and 191, it will be appreciated by those skilled in the art that DNA sequence polymorphisms that lead to changes in the amino acid sequences of the ENDOX polypeptides may exist within a population (e.g., the human population). Such genetic polymorphism in the ENDOX genes may exist among individuals within a population due to natural allelic variation. As used herein, the terms “gene” and “recombinant gene” refer to nucleic acid molecules comprising an open reading frame (ORF) encoding an ENDOX protein, preferably a vertebrate ENDOX protein. Such natural allelic variations can typically result in 1-5% variance in the nucleotide sequence of the ENDOX genes. Any and all such nucleotide variations and resulting amino acid polymorphisms in the ENDOX polypeptides, which are the result of natural allelic variation and that do not alter the functional activity of the ENDOX polypeptides, are intended to be within the scope of the invention.

Moreover, nucleic acid molecules encoding ENDOX proteins from other species, and thus that have a nucleotide sequence that differs from the human sequence of SEQ ID NO: 1, 3, 5, 7, 9, 22, 25, 28, 31, 34, 46, 48, 152, 156, 165, 174, 178, 182, and 191, are intended to be within the scope of the invention. Nucleic acid molecules corresponding to natural allelic variants and homologues of the ENDOX cDNAs of the invention can be isolated based on their homology to the human ENDOX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO: 1, 3, 5, 7, 9, 22, 25, 28, 31, 34, 46, 48, 152, 156, 165, 174, 178, 182, and 191. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding ENDOX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at

pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequences of SEQ ID NO: 1, 3, 5, 7, 9, 22, 25, 28, 31, 34, 46, 48, 152, 156, 165, 174, 178, 182, and 191, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO: 1, 3, 5, 7, 9, 22, 25, 28, 31, 34, 46, 48, 152, 156, 165, 174, 178, 182, and 191, or fragments, analogs or derivatives thereof, under conditions of moderate stringency is provided. A non-limiting example of moderate stringency hybridization conditions are hybridization in 6×SSC, 5× Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Kriegler, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences of SEQ ID NO: 1, 3, 5, 7, 9, 22, 25, 28, 31, 34, 46, 48, 152, 156, 165, 174, 178, 182, and 191, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of ENDOX sequences that may exist in the population, the skilled artisan will further appreciate that changes can be introduced by mutation into the nucleotide sequences of SEQ ID NO: 1, 3, 5, 7, 9, 22, 25, 28, 31, 34, 46, 48, 152, 156, 165, 174, 178, 182, and 191, thereby leading to changes in the amino acid sequences of the encoded ENDOX proteins, without altering the functional ability of said ENDOX proteins. For example, nucleotide substitutions leading to amino acid substitutions at “non-essential” amino acid residues can be made in the sequence of SEQ ID NO:2, 4, 6, 8, 10, 15, 16, 17, 18, 19, 20, 21, 23, 24, 26, 27, 29, 30, 32, 33, 35 to 45 inclusive, 47, 49, 153, 157, 166, 175, 179, 183, 192, and 198. A “non-essential” amino acid residue is a residue that can be altered from the wild-type sequences of the ENDOX proteins without altering their biological activity, whereas an “essential” amino acid residue is required for such biological activity. For example, amino acid residues that are conserved among the ENDOX proteins of the invention are predicted to be particularly non-amenable to alteration. Amino acids for which conservative substitutions can be made are well-known within the art.

Another aspect of the invention pertains to nucleic acid molecules encoding ENDOX proteins that contain changes in amino acid residues that are not essential for activity. Such ENDOX proteins differ in amino acid sequence from SEQ ID NO:2, 4, 6, 8, 10, 15, 16, 17, 18, 19, 20, 21, 23, 24, 26, 27, 29, 30, 32, 33, 35 to 45 inclusive, 47, 49, 153, 157, 166, 175, 179, 183, 192, and 198, yet retain biological activity. In one embodiment, the isolated nucleic acid molecule comprises a nucleotide sequence encoding a protein, wherein the protein comprises an amino acid sequence at least about 45% homologous to the amino acid sequences of SEQ ID NO:2, 4, 6, 8, 10, 15, 16, 17, 18, 19, 20, 21, 23, 24, 26, 27, 29, 30, 32, 33, 35 to 45 inclusive, 47, 49, 153, 157, 166, 175, 179, 183, 192, and 198. Preferably, the protein encoded by the nucleic acid molecule is at least about 60% homologous to SEQ ID NO:2, 4, 6, 8, 10, 15, 16, 17, 18, 19, 20, 21, 23, 24, 26, 27, 29, 30, 32, 33, 35 to 45 inclusive, 47, 49, 153, 157, 166, 175, 179, 183, 192, and 198; more preferably at least about 70% homologous to SEQ ID NO:2, 4, 6, 8, 10, 15, 16, 17, 18, 19, 20, 21, 23, 24, 26, 27, 29, 30, 32, 33, 35 to 45 inclusive, 47, 49, 153, 157, 166, 175, 179, 183, 192, and 198; still more preferably at least about 80% homologous to SEQ ID NO:2, 4, 6, 8, 10, 15, 16, 17, 18, 19, 20, 21, 23, 24, 26, 27, 29, 30, 32, 33, 35 to 45 inclusive, 47, 49, 153, 157, 166, 175, 179, 183, 192, and 198; even more preferably at least about 90% homologous to SEQ ID NO:2, 4, 6, 8, 10, 15, 16, 17, 18, 19, 20, 21, 23, 24, 26, 27, 29, 30, 32, 33, 35 to 45 inclusive, 47, 49, 153, 157, 166, 175, 179, 183, 192, and 198; and most preferably at least about 95% homologous to SEQ ID NO:2, 4, 6, 8, 10, 15, 16, 17, 18, 19, 20, 21, 23, 24, 26, 27, 29, 30, 32, 33, 35 to 45 inclusive, 47, 49, 153, 157, 166, 175, 179, 183, 192, and 198.

An isolated nucleic acid molecule encoding an ENDOX protein homologous to the protein of SEQ ID NO:2, 4, 6, 8, 10, 15, 16, 17, 18, 19, 20, 21, 23, 24, 26, 27, 29, 30, 32, 33, 35 to 45 inclusive, 47, 49, 153, 157, 166, 175, 179, 183, 192, and 198, can be created by introducing one or more nucleotide substitutions, additions or deletions into the nucleotide sequence of SEQ ID NO: 1, 3, 5, 7, 9, 22, 25, 28, 31, 34, 46, 48, 152, 156, 165, 174, 178, 182, and 191, such that one or more amino acid substitutions, additions or deletions are introduced into the encoded protein.

Mutations can be introduced into SEQ ID NO:2, 4, 6, 8, 10, 15, 16, 17, 18, 19, 20, 21, 23, 24, 26, 27, 29, 30, 32, 33, 35 to 45 inclusive, 47, 49, 153, 157, 166, 175, 179, 183, 192, and 198, by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the ENDOX protein is replaced with another amino acid residue from the same side chain family. Alternatively, in another embodiment, mutations can be introduced randomly along all or part of an ENDOX coding sequence, such as by saturation mutagenesis, and the resultant mutants can be screened for ENDOX biological activity to identify mutants that retain activity. Following mutagenesis of SEQ ID NO:2, 4, 6, 8, 10, 15, 16, 17, 18, 19, 20, 21, 23, 24, 26, 27, 29, 30, 32, 33, 35 to 45 inclusive, 47, 49, 153, 157, 166, 175, 179, 183, 192, and 198, the encoded protein can be expressed by any recombinant technology known in the art and the activity of the protein can be determined.

In one embodiment, a mutant ENDOX protein can be assayed for (i) the ability to form protein:protein interactions with other ENDOX proteins, other cell-surface proteins, or biologically-active portions thereof, (ii) complex formation between a mutant ENDOX protein and an ENDOX ligand; or (iii) the ability of a mutant ENDOX protein to bind to an intracellular target protein or biologically-active portion thereof; (e.g. avidin proteins).

In yet another embodiment, a mutant ENDOX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NO: 1, 3, 5, 7, 9, 22, 25, 28, 31, 34, 46, 48, 152, 156, 165, 174, 178, 182, and 191, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire ENDOX coding strand, or to only a portion thereof. Nucleic acid molecules encoding fragments, homologs, derivatives and analogs of an ENDOX protein of SEQ ID NO:2, 4, 6, 8, 10, 15, 16, 17, 18, 19, 20, 21, 23, 24, 26, 27, 29, 30, 32, 33, 35 to 45 inclusive, 47, 49, 153, 157, 166, 175, 179, 183, 192, and 198; or antisense nucleic acids complementary to an ENDOX nucleic acid sequence of SEQ ID NO: 1, 3, 5, 7, 9, 22, 25, 28, 31, 34, 46, 48, 152, 156, 165, 174, 178, 182, and 191, are additionally provided.

In one embodiment, an antisense nucleic acid molecule is antisense to a “coding region” of the coding strand of a nucleotide sequence encoding an ENDOX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the ENDOX protein. The term “noncoding region” refers to 5′ and 3′ sequences which flank the coding region that are not translated into amino acids (i.e., also referred to as 5′ and 3′ untranslated regions).

Given the coding strand sequences encoding the ENDOX protein disclosed herein, antisense nucleic acids of the invention can be designed according to the rules of Watson and Crick or Hoogsteen base pairing. The antisense nucleic acid molecule can be complementary to the entire coding region of ENDOX mRNA, but more preferably is an oligonucleotide that is antisense to only a portion of the coding or noncoding region of ENDOX mRNA. For example, the antisense oligonucleotide can be complementary to the region surrounding the translation start site of ENDOX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an ENDOX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (see, e.g., Inoue, et al. 1987. Nucl. Acids Res. 15: 6131-6148) or a chimeric RNA-DNA analogue (see, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave ENDOX mRNA transcripts to thereby inhibit translation of ENDOX mRNA. A ribozyme having specificity for an ENDOX-encoding nucleic acid can be designed based upon the nucleotide sequence of an ENDOX cDNA disclosed herein (i.e., SEQ ID NO: 1, 3, 5, 7, 9, 22, 25, 28, 31, 34, 46, 48, 152, 156, 165, 174, 178, 182, and 191). For example, a derivative of a Tetrahymena L-19 IVS RNA can be constructed in which the nucleotide sequence of the active site is complementary to the nucleotide sequence to be cleaved in an ENDOX-encoding mRNA. See, e.g., U.S. Pat. No. 4,987,071 to Cech, et al. and U.S. Pat. No. 5,116,742 to Cech, et al. ENDOX mRNA can also be used to select a catalytic RNA having a specific ribonuclease activity from a pool of RNA molecules. See, e.g., Bartel et al., (1993) Science 261:1411-1418.

Alternatively, ENDOX gene expression can be inhibited by targeting nucleotide sequences complementary to the regulatory region of the ENDOX nucleic acid (e.g., the ENDOX promoter and/or enhancers) to form triple helical structures that prevent transcription of the ENDOX gene in target cells. See, e.g., Helene, 1991. Anticancer Drug Des. 6: 569-84; Helene, et al. 1992. Ann. NAY. Acad. Sci. 660: 27-36; Maher, 1992. Bioassays 14: 807-15.

In various embodiments, the ENDOX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of ENDOX can be used in therapeutic and diagnostic applications. For example, PNAs can be used as antisense or antigene agents for sequence-specific modulation of gene expression by, e.g., inducing transcription or translation arrest or inhibiting replication. PNAs of ENDOX can also be used, for example, in the analysis of single base pair mutations in a gene (e.g., PNA directed PCR clamping; as artificial restriction enzymes when used in combination with other enzymes, e.g., S₁ nucleases (see, Hyrup, et al., 1996. supra); or as probes or primers for DNA sequence and hybridization (see, Hyrup, et al., 1996, supra; Perry-O'Keefe, et al., 1996. supra).

In another embodiment, PNAs of ENDOX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of ENDOX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

ENDOX Polypeptides

A polypeptide according to the invention includes a polypeptide including the amino acid sequence of ENDOX polypeptides whose sequences are provided in SEQ ID NO:2, 4, 6, 8, 10, 15, 16, 17, 18, 19, 20, 21, 23, 24, 26, 27, 29, 30, 32, 33, 35 to 45 inclusive, 47, 49, 153, 157, 166, 175, 179, 183, 192, and 198. The invention also includes a mutant or variant protein any of whose residues may be changed from the corresponding residues shown in SEQ ID NO:2, 4, 6, 8, 10, 15, 16, 17, 18, 19, 20, 21, 23, 24, 26, 27, 29, 30, 32, 33, 35 to 45 inclusive, 47, 49, 153, 157, 166, 175, 179, 183, 192, and 198, while still encoding a protein that maintains its ENDOX activities and physiological functions, or a functional fragment thereof.

In general, an ENDOX variant that preserves ENDOX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated ENDOX proteins, and biologically-active portions thereof, or derivatives, fragments, analogs or homologs thereof. Also provided are polypeptide fragments suitable for use as immunogens to raise anti-ENDOX antibodies. In one embodiment, native ENDOX proteins can be isolated from cells or tissue sources by an appropriate purification scheme using standard protein purification techniques. In another embodiment, ENDOX proteins are produced by recombinant DNA techniques. Alternative to recombinant expression, an ENDOX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the ENDOX protein is derived, or substantially free from chemical precursors or other chemicals when chemically synthesized. The language “substantially free of cellular material” includes preparations of ENDOX proteins in which the protein is separated from cellular components of the cells from which it is isolated or recombinantly-produced. In one embodiment, the language “substantially free of cellular material” includes preparations of ENDOX proteins having less than about 30% (by dry weight) of non-ENDOX proteins (also referred to herein as a “contaminating protein”), more preferably less than about 20% of non-ENDOX proteins, still more preferably less than about 10% of non-ENDOX proteins, and most preferably less than about 5% of non-ENDOX proteins. When the ENDOX protein or biologically-active portion thereof is recombinantly-produced, it is also preferably substantially free of culture medium, i.e., culture medium represents less than about 20%, more preferably less than about 10%, and most preferably less than about 5% of the volume of the ENDOX protein preparation.

The language “substantially free of chemical precursors or other chemicals” includes preparations of ENDOX proteins in which the protein is separated from chemical precursors or other chemicals that are involved in the synthesis of the protein. In one embodiment, the language “substantially free of chemical precursors or other chemicals” includes preparations of ENDOX proteins having less than about 30% (by dry weight) of chemical precursors or non-ENDOX chemicals, more preferably less than about 20% chemical precursors or non-ENDOX chemicals, still more preferably less than about 10% chemical precursors or non-ENDOX chemicals, and most preferably less than about 5% chemical precursors or non-ENDOX chemicals.

Biologically-active portions of ENDOX proteins include peptides comprising amino acid sequences sufficiently homologous to or derived from the amino acid sequences of the ENDOX proteins (e.g., the amino acid sequence shown in SEQ ID NO:2, 4, 6, 8, 10, 15, 16, 17, 18, 19, 20, 21, 23, 24, 26, 27, 29, 30, 32, 33, 35 to 45 inclusive, 47, 49, 153, 157, 166, 175, 179, 183, 192, and 198) that include fewer amino acids than the full-length ENDOX proteins, and exhibit at least one activity of an ENDOX protein. Typically, biologically-active portions comprise a domain or motif with at least one activity of the ENDOX protein. A biologically-active portion of an ENDOX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native ENDOX protein.

In an embodiment, the ENDOX protein has an amino acid sequence shown in SEQ ID NO:2, 4, 6, 8, 10, 15, 16, 17, 18, 19, 20, 21, 23, 24, 26, 27, 29, 30, 32, 33, 35 to 45 inclusive, 47, 49, 153, 157, 166, 175, 179, 183, 192, and 198. In other embodiments, the ENDOX protein is substantially homologous to SEQ ID NO:2, 4, 6, 8, 10, 15, 16, 17, 18, 19, 20, 21, 23, 24, 26, 27, 29, 30, 32, 33, 35 to 45 inclusive, 47, 49, 153, 157, 166, 175, 179, 183, 192, and 198, and retains the functional activity of the protein of SEQ ID NO:2, 4, 6, 8, 10, 15, 16, 17, 18, 19, 20, 21, 23, 24, 26, 27, 29, 30, 32, 33, 35 to 45 inclusive, 47, 49, 153, 157, 166, 175, 179, 183, 192, and 198, yet differs in amino acid sequence due to natural allelic variation or mutagenesis, as described in detail, below. Accordingly, in another embodiment, the ENDOX protein is a protein that comprises an amino acid sequence at least about 45% homologous to the amino acid sequence of SEQ ID NO:2, 4, 6, 8, 10, 15, 16, 17, 18, 19, 20, 21, 23, 24, 26, 27, 29, 30, 32, 33, 35 to 45 inclusive, 47, 49, 153, 157, 166, 175, 179, 183, 192, and 198 and retains the functional activity of the ENDOX proteins of SEQ ID NO:2, 4, 6, 8, 10, 15, 16, 17, 18, 19, 20, 21, 23, 24, 26, 27, 29, 30, 32, 33, 35 to 45 inclusive, 47, 49, 153, 157, 166, 175, 179, 183, 192, and 198.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (ire., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NO: 1, 3, 5, 7, 9, 22, 25, 28, 31, 34, 46, 48, 152, 156, 165, 174, 178, 182, and 191.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides ENDOX chimeric or fusion proteins. As used herein, an ENDOX “chimeric protein” or “fusion protein” comprises an ENDOX polypeptide operatively-linked to a non-ENDOX polypeptide. An “ENDOX polypeptide” refers to a polypeptide having an amino acid sequence corresponding to an ENDOX protein (SEQ ID NO:2, 4, 6, 8, 10, 15, 16, 17, 18, 19, 20, 21, 23, 24, 26, 27, 29, 30, 32, 33, 35 to 45 inclusive, 47, 49, 153, 157, 166, 175, 179, 183, 192, and 198), whereas a “non-ENDOX polypeptide” refers to a polypeptide having an amino acid sequence corresponding to a protein that is not substantially homologous to the ENDOX protein, e.g., a protein that is different from the ENDOX protein and that is derived from the same or a different organism. Within an ENDOX fusion protein the ENDOX polypeptide can correspond to all or a portion of an ENDOX protein. In one embodiment, an ENDOX fusion protein comprises at least one biologically-active portion of an ENDOX protein. In another embodiment, an ENDOX fusion protein comprises at least two biologically-active portions of an ENDOX protein. In yet another embodiment, an ENDOX fusion protein comprises at least three biologically-active portions of an ENDOX protein. Within the fusion protein, the term “operatively-linked” is intended to indicate that the ENDOX polypeptide and the non-ENDOX polypeptide are fused in-frame with one another. The non-ENDOX polypeptide can be fused to the N-terminus or C-terminus of the ENDOX polypeptide.

In one embodiment, the fusion protein is a GST-ENDOX fusion protein in which the ENDOX sequences are fused to the C-terminus of the GST (glutathione S-transferase) sequences. Such fusion proteins can facilitate the purification of recombinant ENDOX polypeptides.

In another embodiment, the fusion protein is an ENDOX protein containing a heterologous signal sequence at its N-terminus. In certain host cells (e.g., mammalian host cells), expression and/or secretion of ENDOX can be increased through use of a heterologous signal sequence.

In yet another embodiment, the fusion protein is an ENDOX-immunoglobulin fusion protein in which the ENDOX sequences are fused to sequences derived from a member of the immunoglobulin protein family. The ENDOX-immunoglobulin fusion proteins of the invention can be incorporated into pharmaceutical compositions and administered to a subject to inhibit an interaction between an ENDOX ligand and an ENDOX protein on the surface of a cell, to thereby suppress ENDOX-mediated signal transduction in vivo. The ENDOX-immunoglobulin fusion proteins can be used to affect the bioavailability of an ENDOX cognate ligand. Inhibition of the ENDOX ligand/ENDOX interaction may be useful therapeutically for both the treatment of proliferative and differentiative disorders, as well as modulating (e.g. promoting or inhibiting) cell survival. Moreover, the ENDOX-immunoglobulin fusion proteins of the invention can be used as immunogens to produce anti-ENDOX antibodies in a subject, to purify ENDOX ligands, and in screening assays to identify molecules that inhibit the interaction of ENDOX with an ENDOX ligand.

An ENDOX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate term in, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An ENDOX-encoding nucleic acid can be cloned into such an expression vector such that the fusion moiety is linked in-frame to the ENDOX protein.

ENDOX Agonists and Antagonists

The invention also pertains to variants of the ENDOX proteins that function as either ENDOX agonists (i.e., mimetics) or as ENDOX antagonists. Variants of the ENDOX protein can be generated by mutagenesis (e.g., discrete point mutation or truncation of the ENDOX protein). An agonist of the ENDOX protein can retain substantially the same, or a subset of, the biological activities of the naturally occurring form of the ENDOX protein. An antagonist of the ENDOX protein can inhibit one or more of the activities of the naturally occurring form of the ENDOX protein by, for example, competitively binding to a downstream or upstream member of a cellular signaling cascade which includes the ENDOX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the ENDOX proteins.

Variants of the ENDOX proteins that function as either ENDOX agonists (i.e., mimetics) or as ENDOX antagonists can be identified by screening combinatorial libraries of mutants (e.g., truncation mutants) of the ENDOX proteins for ENDOX protein agonist or antagonist activity. In one embodiment, a variegated library of ENDOX variants is generated by combinatorial mutagenesis at the nucleic acid level and is encoded by a variegated gene library. A variegated library of ENDOX variants can be produced by, for example, enzymatically ligating a mixture of synthetic oligonucleotides into gene sequences such that a degenerate set of potential ENDOX sequences is expressible as individual polypeptides, or alternatively, as a set of larger fusion proteins (e.g., for phage display) containing the set of ENDOX sequences therein. There are a variety of methods which can be used to produce libraries of potential ENDOX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential ENDOX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the ENDOX protein coding sequences can be used to generate a variegated population of ENDOX fragments for screening and subsequent selection of variants of an ENDOX protein. In one embodiment, a library of coding sequence fragments can be generated by treating a double stranded PCR fragment of an ENDOX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the ENDOX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of ENDOX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify ENDOX variants. See, e.g., Arkin and Yourvan, 1992. Proc. Natl. Acad. Sci. USA 89: 7811-7815; Delgrave, et al., 1993. Protein Engineering 6:327-331.

Anti-ENDOX Antibodies

The invention encompasses antibodies and antibody fragments, such as F_{ab} or (F_{ab})₂ that bind immunospecifically to any of the ENDOX polypeptides of said invention.

An isolated ENDOX protein, or a portion or fragment thereof, can be used as an immunogen to generate antibodies that bind to ENDOX polypeptides using standard techniques for polyclonal and monoclonal antibody preparation. The full-length ENDOX proteins can be used or, alternatively, the invention provides antigenic peptide fragments of ENDOX proteins for use as immunogens. The antigenic ENDOX peptides comprises at least 4 amino acid residues of the amino acid sequence shown in SEQ ID NO:2, 4, 6, 8, 10, 15, 16, 17, 18, 19, 20, 21, 23, 24, 26, 27, 29, 30, 32, 33, 35 to 45 inclusive, 47, 49, 153, 157, 166, 175, 179, 183, 192, and 198, and encompasses an epitope of ENDOX such that an antibody raised against the peptide forms a specific immune complex with ENDOX. Preferably, the antigenic peptide comprises at least 6, 8, 10, 15, 20, or 30 amino acid residues. Longer antigenic peptides are sometimes preferable over shorter antigenic peptides, depending on use and according to methods well known to someone skilled in the art.

In certain embodiments of the invention, at least one epitope encompassed by the antigenic peptide is a region of ENDOX that is located on the surface of the protein (e.g., a hydrophilic region). As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation (see, e.g., Hopp and Woods, 1981. Proc. Nat. Acad. Sci. USA 78: 3824-3828; Kyte and Doolittle, 1982. J. Mol. Biol. 157: 105-142, each incorporated herein by reference in their entirety).

As disclosed herein, ENDOX protein sequences of SEQ ID NO:2, 4, 6, 8, 10, or derivatives, fragments, analogs or homologs thereof, may be utilized as immunogens in the generation of antibodies that immunospecifically-bind these protein components. The term “antibody” as used herein refers to immunoglobulin molecules and immunologically-active portions of immunoglobulin molecules, i.e., molecules that contain an antigen binding site that specifically-binds (immunoreacts with) an antigen, such as ENDOX. Such antibodies include, but are not limited to, polyclonal, monoclonal, chimeric, single chain, F_{ab} and F_{(ab′)2} fragments, and an F_{ab} expression library. In a specific embodiment, antibodies to human ENDOX proteins are disclosed. Various procedures known within the art may be used for the production of polyclonal or monoclonal antibodies to an ENDOX protein sequence of SEQ ID NO:2, 4, 6, 8, 10, or a derivative, fragment, analog or homolog thereof. Some of these proteins are discussed below.

For the production of polyclonal antibodies, various suitable host animals (e.g., rabbit, goat, mouse or other mammal) may be immunized by injection with the native protein, or a synthetic variant thereof, or a derivative of the foregoing. An appropriate immunogenic preparation can contain, for example, recombinantly-expressed ENDOX protein or a chemically-synthesized ENDOX polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against ENDOX can be isolated from the mammal (e.g., from the blood) and further purified by well known techniques, such as protein A chromatography to obtain the IgG fraction.

The term “monoclonal antibody” or “monoclonal antibody composition”, as used herein, refers to a population of antibody molecules that contain only one species of an antigen binding site capable of immunoreacting with a particular epitope of ENDOX. A monoclonal antibody composition thus typically displays a single binding affinity for a particular ENDOX protein with which it immunoreacts. For preparation of monoclonal antibodies directed towards a particular ENDOX protein, or derivatives, fragments, analogs or homologs thereof, any technique that provides for the production of antibody molecules by continuous cell line culture may be utilized. Such techniques include, but are not limited to, the hybridoma technique (see, e.g., Kohler & Milstein, 1975. Nature 256: 495497); the trioma technique; the human B-cell hybridoma technique (see, e.g., Kozbor, et al., 1983. Immunol. Today 4: 72) and the EBV hybridoma technique to produce human monoclonal antibodies (see, e.g., Cole, et al., 1985. In: MONOCLONAL ANTIBODIES AND CANCER THERAPY, Alan R. Liss, Inc., pp. 77-96). Human monoclonal antibodies may be utilized in the practice of the invention and may be produced by using human hybridomas (see, e.g., Cote, et al., 1983. Proc Natl Acad Sci USA 80: 2026-2030) or by transforming human B-cells with Epstein Barr Virus in vitro (see, e.g., Cole, et al., 1985. In: MONOCLONAL ANTIBODIES AND CANCER THERAPY, Alan R. Liss, Inc., pp. 77-96). Each of the above citations is incorporated herein by reference in their entirety.

According to the invention, techniques can be adapted for the production of single-chain antibodies specific to an ENDOX protein (see, e.g., U.S. Pat. No. 4,946,778). In addition, methods can be adapted for the construction of F_{ab} expression libraries (see, e.g., Huse, et al., 1989. Science 246: 1275-1281) to allow rapid and effective identification of monoclonal F_{ab} fragments with the desired specificity for an ENDOX protein or derivatives, fragments, analogs or homologs thereof. Non-human antibodies can be “humanized” by techniques well known in the art. See, e.g., U.S. Pat. No. 5,225,539. Antibody fragments that contain the idiotypes to an ENDOX protein may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent; and (iv) Fᵥ fragments.

Additionally, recombinant anti-ENDOX antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in International Application No. PCT/US86/02269; European Patent Application No. 184,187; European Patent Application No. 171,496; European Patent Application No. 173,494; PCT International Publication No. WO 86/01533; U.S. Pat. No. 4,816,567; U.S. Pat. No. 5,225,539; European Patent Application No. 125,023; Better, et al., 1988. Science 240: 1041-1043; Liu, et al., 1987. Proc. Natl. Acad. Sci. USA 84: 3439-3443; Liu, et al., 1987. J. Immunol. 139: 3521-3526; Sun, et al., 1987. Proc. Natl. Acad. Sci. USA 84: 214-218; Nishimura, et al., 1987. Cancer Res. 47: 999-1005; Wood, et al., 1985. Nature 314: 446-449; Shaw, et al., 1988. J. Natl. Cancer Inst. 80: 1553-1559); Morrison (1985) Science 229:1202-1207; Oi, et al. (1986) BioTechniques 4:214; Jones, et al., 1986. Nature 321: 552-525; Verhoeyan, et al., 1988. Science 239: 1534; and Beidler, et al., 1988. J. Immunol. 141: 4053-4060. Each of the above citations are incorporated herein by reference in their entirety.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an ENDOX protein is facilitated by generation of hybridomas that bind to the fragment of an ENDOX protein possessing such a domain. Thus, antibodies that are specific for a desired domain within an ENDOX protein, or derivatives, fragments, analogs or homologs thereof, are also provided herein.

Anti-ENDOX antibodies may be used in methods known within the art relating to the localization and/or quantitation of an ENDOX protein (e.g., for use in measuring levels of the ENDOX protein within appropriate physiological samples, for use in diagnostic methods, for use in imaging the protein, and the like). In a given embodiment, antibodies for ENDOX proteins, or derivatives, fragments, analogs or homologs thereof, that contain the antibody derived binding domain, are utilized as pharmacologically-active compounds (hereinafter “Therapeutics”).

An anti-ENDOX antibody (e.g., monoclonal antibody) can be used to isolate an ENDOX polypeptide by standard techniques, such as affinity chromatography or immunoprecipitation. An anti-ENDOX antibody can facilitate the purification of natural ENDOX polypeptide from cells and of recombinantly-produced ENDOX polypeptide expressed in host cells. Moreover, an anti-ENDOX antibody can be used to detect ENDOX protein (e.g., in a cellular lysate or cell supernatant) in order to evaluate the abundance and pattern of expression of the ENDOX protein. Anti-ENDOX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

ENDOX Recombinant Expression Vectors and Host Cells

Another aspect of the invention pertains to vectors, preferably expression vectors, containing a nucleic acid encoding an ENDOX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., ENDOX proteins, mutant forms of ENDOX proteins, fusion proteins, etc.).

The recombinant expression vectors of the invention can be designed for expression of ENDOX proteins in prokaryotic or eukaryotic cells. For example, ENDOX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the ENDOX expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, ENDOX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to ENDOX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, ENDOX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding ENDOX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) ENDOX protein. Accordingly, the invention further provides methods for producing ENDOX protein using the host cells of the invention. In one embodiment, the method comprises culturing the host cell of invention (into which a recombinant expression vector encoding ENDOX protein has been introduced) in a suitable medium such that ENDOX protein is produced. In another embodiment, the method further comprises isolating ENDOX protein from the medium or the host cell.

Transgenic ENDOX Animals

The host cells of the invention can also be used to produce non-human transgenic animals. For example, in one embodiment, a host cell of the invention is a fertilized oocyte or an embryonic stem cell into which ENDOX protein-coding sequences have been introduced. Such host cells can then be used to create non-human transgenic animals in which exogenous ENDOX sequences have been introduced into their genome or homologous recombinant animals in which endogenous ENDOX sequences have been altered. Such animals are useful for studying the function and/or activity of ENDOX protein and for identifying and/or evaluating modulators of ENDOX protein activity. As used herein, a “transgenic animal” is a IDS non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous ENDOX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing ENDOX-encoding nucleic acid into the male pronuclei of a fertilized oocyte (e.g., by microinjection, retroviral infection) and allowing the oocyte to develop in a pseudopregnant female foster animal. The human ENDOX cDNA sequences of SEQ ID NO: 1, 3, 5, 7, 9, 22, 25, 28, 31, 34, 46, 48, 152, 156, 165, 174, 178, 182, and 191, can be introduced as a transgene into the genome of a non-human animal. Alternatively, a non-human homologue of the human ENDOX gene, such as a mouse ENDOX gene, can be isolated based on hybridization to the human ENDOX cDNA (described further supra) and used as a transgene. Intronic sequences and polyadenylation signals can also be included in the transgene to increase the efficiency of expression of the transgene. A tissue-specific regulatory sequence(s) can be operably-linked to the ENDOX transgene to direct expression of ENDOX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the ENDOX transgene in its genome and/or expression of ENDOX mRNA in tissues or cells of the animals. A transgenic founder animal can then be used to breed additional animals carrying the transgene. Moreover, transgenic animals carrying a transgene-encoding ENDOX protein can further be bred to other transgenic animals carrying other transgenes.

To create a homologous recombinant animal, a vector is prepared which contains at least a portion of an ENDOX gene into which a deletion, addition or substitution has been introduced to thereby alter, e.g., functionally disrupt, the ENDOX gene. The ENDOX gene can be a human gene (e.g., the cDNA of SEQ ID NO:1, 3, 5, 7, 9, 22, 25, 28, 31, 34, 46, 48, 152, 156, 165, 174, 178, 182, and 191), but more preferably, is a non-human homologue of a human ENDOX gene. For example, a mouse homologue of human ENDOX gene of SEQ ID NO:1, 3, 5, 7, 9, 22, 25, 28, 31, 34, 46, 48, 152, 156, 165, 174, 178, 182, and 191, can be used to construct a homologous recombination vector suitable for altering an endogenous ENDOX gene in the mouse genome. In one embodiment, the vector is designed such that, upon homologous recombination, the endogenous ENDOX gene is functionally disrupted (i.e., no longer encodes a functional protein; also referred to as a “knock out” vector).

Alternatively, the vector can be designed such that, upon homologous recombination, the endogenous ENDOX gene is mutated or otherwise altered but still encodes functional protein (e.g., the upstream regulatory region can be altered to thereby alter the expression of the endogenous ENDOX protein). In the homologous recombination vector, the altered portion of the ENDOX gene is flanked at its 5′- and 3′-termini by additional nucleic acid of the ENDOX gene to allow for homologous recombination to occur between the exogenous ENDOX gene carried by the vector and an endogenous ENDOX gene in an embryonic stem cell. The additional flanking ENDOX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced ENDOX gene has homologously-recombined with the endogenous ENDOX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The ENDOX nucleic acid molecules, ENDOX proteins, and anti-ENDOX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an ENDOX protein or anti-ENDOX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express ENDOX protein (e.g., via a recombinant expression vector in a host cell in gene therapy applications), to detect ENDOX mRNA (e.g., in a biological sample) or a genetic lesion in an ENDOX gene, and to modulate ENDOX activity, as described further, below. In addition, the ENDOX proteins can be used to screen drugs or compounds that modulate the ENDOX protein activity or expression as well as to treat disorders characterized by insufficient or excessive production of ENDOX protein or production of ENDOX protein forms that have decreased or aberrant activity compared to ENDOX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease (possesses anti-microbial activity) and the various dyslipidemias. In addition, the anti-ENDOX antibodies of the invention can be used to detect and isolate ENDOX proteins and modulate ENDOX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to ENDOX proteins or have a stimulatory or inhibitory effect on, e.g., ENDOX protein expression or ENDOX protein activity. The invention also includes compounds identified in the screening assays described herein.

In one embodiment, the invention provides assays for screening candidate or test compounds which bind to or modulate the activity of the membrane-bound form of an ENDOX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12:145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37:1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of ENDOX protein, or a biologically-active portion thereof, on the cell surface is contacted with a test compound and the ability of the test compound to bind to an ENDOX protein determined. The cell, for example, can of mammalian origin or a yeast cell. Determining the ability of the test compound to bind to the ENDOX protein can be accomplished, for example, by coupling the test compound with a radioisotope or enzymatic label such that binding of the test compound to the ENDOX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of ENDOX protein, or a biologically-active portion thereof, on the cell surface with a known compound which binds ENDOX to form an assay mixture, contacting the assay mixture with a test compound, and determining the ability of the test compound to interact with an ENDOX protein, wherein determining the ability of the test compound to interact with an ENDOX protein comprises determining the ability of the test compound to preferentially bind to ENDOX protein or a biologically-active portion thereof as compared to the known compound.

In another embodiment, an assay is a cell-based assay comprising contacting a cell expressing a membrane-bound form of ENDOX protein, or a biologically-active portion thereof, on the cell surface with a test compound and determining the ability of the test compound to modulate (e.g., stimulate or inhibit) the activity of the ENDOX protein or biologically-active portion thereof. Determining the ability of the test compound to modulate the activity of ENDOX or a biologically-active portion thereof can be accomplished, for example, by determining the ability of the ENDOX protein to bind to or interact with an ENDOX target molecule. As used herein, a “target molecule” is a molecule with which an ENDOX protein binds or interacts in nature, for example, a molecule on the surface of a cell which expresses an ENDOX interacting protein, a molecule on the surface of a second cell, a molecule in the extracellular milieu, a molecule associated with the internal surface of a cell membrane or a cytoplasmic molecule. An ENDOX target molecule can be a non-ENDOX molecule or an ENDOX protein or polypeptide of the invention. In one embodiment, an ENDOX target molecule is a component of a signal transduction pathway that facilitates transduction of an extracellular signal (e.g. a signal generated by binding of a compound to a membrane-bound ENDOX molecule) through the cell membrane and into the cell. The target, for example, can be a second intercellular protein that has catalytic activity or a protein that facilitates the association of downstream signaling molecules with ENDOX.

Determining the ability of the ENDOX protein to bind to or interact with an ENDOX target molecule can be accomplished by one of the methods described above for determining direct binding. In one embodiment, determining the ability of the ENDOX protein to bind to or interact with an ENDOX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an ENDOX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an ENDOX protein or biologically-active portion thereof with a test compound and determining the ability of the test compound to bind to the ENDOX protein or biologically-active portion thereof. Binding of the test compound to the ENDOX protein can be determined either directly or indirectly as described above. In one such embodiment, the assay comprises contacting the ENDOX protein or biologically-active portion thereof with a known compound which binds ENDOX to form an assay mixture, contacting the assay mixture with a test compound, and determining the ability of the test compound to interact with an ENDOX protein, wherein determining the ability of the test compound to interact with an ENDOX protein comprises determining the ability of the test compound to preferentially bind to ENDOX or biologically-active portion thereof as compared to the known compound.

In still another embodiment, an assay is a cell-free assay comprising contacting ENDOX protein or biologically-active portion thereof with a test compound and determining the ability of the test compound to modulate (e.g. stimulate or inhibit) the activity of the ENDOX protein or biologically-active portion thereof. Determining the ability of the test compound to modulate the activity of ENDOX can be accomplished, for example, by determining the ability of the ENDOX protein to bind to an ENDOX target molecule by one of the methods described above for determining direct binding. In an alternative embodiment, determining the ability of the test compound to modulate the activity of ENDOX protein can be accomplished by determining the ability of the ENDOX protein further modulate an ENDOX target molecule. For example, the catalytic/enzymatic activity of the target molecule on an appropriate substrate can be determined as described, supra.

In yet another embodiment, the cell-free assay comprises contacting the ENDOX protein or biologically-active portion thereof with a known compound which binds ENDOX protein to form an assay mixture, contacting the assay mixture with a test compound, and determining the ability of the test compound to interact with an ENDOX protein, wherein determining the ability of the test compound to interact with an ENDOX protein comprises determining the ability of the ENDOX protein to preferentially bind to or modulate the activity of an ENDOX target molecule.

The cell-free assays of the invention are amenable to use of both the soluble form or the membrane-bound form of ENDOX protein. In the case of cell-free assays comprising the membrane-bound form of ENDOX protein, it may be desirable to utilize a solubilizing agent such that the membrane-bound form of ENDOX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl—N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either ENDOX protein or its target molecule to facilitate separation of complexed from uncomplexed forms of one or both of the proteins, as well as to accommodate automation of the assay. Binding of a test compound to ENDOX protein, or interaction of ENDOX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-ENDOX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or ENDOX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of ENDOX protein binding or activity determined using standard techniques.

Other techniques for immobilizing proteins on matrices can also be used in the screening assays of the invention. For example, either the ENDOX protein or its target molecule can be immobilized utilizing conjugation of biotin and streptavidin. Biotinylated ENDOX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with ENDOX protein or target molecules, but which do not interfere with binding of the ENDOX protein to its target molecule, can be derivatized to the wells of the plate, and unbound target or ENDOX protein trapped in the wells by antibody conjugation. Methods for detecting such complexes, in addition to those described above for the GST-immobilized complexes, include immunodetection of complexes using antibodies reactive with the ENDOX protein or target molecule, as well as enzyme-linked assays that rely on detecting an enzymatic activity associated with the ENDOX protein or target molecule.

In another embodiment, modulators of ENDOX protein expression are identified in a method wherein a cell is contacted with a candidate compound and the expression of ENDOX mRNA or protein in the cell is determined. The level of expression of ENDOX mRNA or protein in the presence of the candidate compound is compared to the level of expression of ENDOX mRNA or protein in the absence of the candidate compound. The candidate compound can then be identified as a modulator of ENDOX mRNA or protein expression based upon this comparison. For example, when expression of ENDOX mRNA or protein is greater (i.e., statistically significantly greater) in the presence of the candidate compound than in its absence, the candidate compound is identified as a stimulator of ENDOX mRNA or protein expression. Alternatively, when expression of ENDOX mRNA or protein is less (statistically significantly less) in the presence of the candidate compound than in its absence, the candidate compound is identified as an inhibitor of ENDOX mRNA or protein expression. The level of ENDOX mRNA or protein expression in the cells can be determined by methods described herein for detecting ENDOX mRNA or protein.

In yet another aspect of the invention, the ENDOX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with ENDOX (“ENDOX-binding proteins” or “ENDOX-bp”) and modulate ENDOX activity. Such ENDOX-binding proteins are also likely to be involved in the propagation of signals by the ENDOX proteins as, for example, upstream or downstream elements of the ENDOX pathway.

The two-hybrid system is based on the modular nature of most transcription factors, which consist of separable DNA-binding and activation domains. Briefly, the assay utilizes two different DNA constructs. In one construct, the gene that codes for ENDOX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an ENDOX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with ENDOX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the ENDOX sequences, SEQ ID NO: 1, 3, 5, 7, 9, 22, 25, 28, 31, 34, 46, 48, 152, 156, 165, 174, 178, 182, and 191, or fragments or derivatives thereof, can be used to map the location of the ENDOX genes, respectively, on a chromosome. The mapping of the ENDOX sequences to chromosomes is an important first step in correlating these sequences with genes associated with disease.

Briefly, ENDOX genes can be mapped to chromosomes by preparing PCR primers (preferably 15-25 bp in length) from the ENDOX sequences. Computer analysis of the ENDOX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the ENDOX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the ENDOX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the ENDOX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The ENDOX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the ENDOX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The ENDOX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NO:1, 3, 5, 7, 9, 22, 25, 28, 31, 34, 46, 48, 152, 156, 165, 174, 178, 182, and 191, are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining ENDOX protein and/or nucleic acid expression as well as ENDOX activity, in the context of a biological sample (e.g., blood, serum, cells, tissue) to thereby determine whether an individual is afflicted with a disease or disorder, or is at risk of developing a disorder, associated with aberrant ENDOX expression or activity. The disorders include metabolic disorders, diabetes, obesity, infectious disease, anorexia, cancer-associated cachexia, and the various dyslipidemias., metabolic disturbances associated with obesity, the metabolic syndrome X and wasting disorders associated with chronic diseases and various cancers. The invention also provides for prognostic (or predictive) assays for determining whether an individual is at risk of developing a disorder associated with ENDOX protein, nucleic acid expression or activity. For example, mutations in an ENDOX gene can be assayed in a biological sample. Such assays can be used for prognostic or predictive purpose to thereby prophylactically treat an individual prior to the onset of a disorder characterized by or associated with ENDOX protein, nucleic acid expression, or biological activity.

Another aspect of the invention provides methods for determining ENDOX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of ENDOX in clinical trials.

These and other agents are described in further detail in the following sections.

Diagnostic Assays

An exemplary method for detecting the presence or absence of ENDOX in a biological sample involves obtaining a biological sample from a test subject and contacting the biological sample with a compound or an agent capable of detecting ENDOX protein or nucleic acid (erg., mRNA, genomic DNA) that encodes ENDOX protein such that the presence of ENDOX is detected in the biological sample. An agent for detecting ENDOX mRNA or genomic DNA is a labeled nucleic acid probe capable of hybridizing to ENDOX mRNA or genomic DNA. The nucleic acid probe can be, for example, a full-length ENDOX nucleic acid, such as the nucleic acid of SEQ ID NO: 1, 3, 5, 7, 9, 22, 25, 28, 31, 34, 46, 48, 152, 156, 165, 174, 178, 182, and 191, or a portion thereof, such as an oligonucleotide of at least 15, 30, 50, 100, 250 or 500 nucleotides in length and sufficient to specifically hybridize under stringent conditions to ENDOX mRNA or genomic DNA. Other suitable probes for use in the diagnostic assays of the invention are described herein.

An agent for detecting ENDOX protein is an antibody capable of binding to ENDOX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect ENDOX mRNA, protein, or genomic DNA in a biological sample in vitro as well as in vivo. For example, in vitro techniques for detection of ENDOX mRNA include Northern hybridizations and in situ hybridizations. In vitro techniques for detection of ENDOX protein include enzyme linked immunosorbent assays (ELISAs), Western blots, immunoprecipitations, and immunofluorescence. In vitro techniques for detection of ENDOX genomic DNA include Southern hybridizations. Furthermore, in vivo techniques for detection of ENDOX protein include introducing into a subject a labeled anti-ENDOX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting ENDOX protein, mRNA, or genomic DNA, such that the presence of ENDOX protein, mRNA or genomic DNA is detected in the biological sample, and comparing the presence of ENDOX protein, mRNA or genomic DNA in the control sample with the presence of ENDOX protein, mRNA or genomic DNA in the test sample.

The invention also encompasses kits for detecting the presence of ENDOX in a biological sample. For example, the kit can comprise: a labeled compound or agent capable of detecting ENDOX protein or mRNA in a biological sample; means for determining the amount of ENDOX in the sample; and means for comparing the amount of ENDOX in the sample with a standard. The compound or agent can be packaged in a suitable container. The kit can further comprise instructions for using the kit to detect ENDOX protein or nucleic acid.

Prognostic Assays

The diagnostic methods described herein can furthermore be utilized to identify subjects having or at risk of developing a disease or disorder associated with aberrant ENDOX expression or activity. For example, the assays described herein, such as the preceding diagnostic assays or the following assays, can be utilized to identify a subject having or at risk of developing a disorder associated with ENDOX protein, nucleic acid expression or activity. Alternatively, the prognostic assays can be utilized to identify a subject having or at risk for developing a disease or disorder. Thus, the invention provides a method for identifying a disease or disorder associated with aberrant ENDOX expression or activity in which a test sample is obtained from a subject and ENDOX protein or nucleic acid (e.g., mRNA, genomic DNA) is detected, wherein the presence of ENDOX protein or nucleic acid is diagnostic for a subject having or at risk of developing a disease or disorder associated with aberrant ENDOX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant ENDOX expression or activity. For example, such methods can be used to determine whether a subject can be effectively treated with an agent for a disorder. Thus, the invention provides methods for determining whether a subject can be effectively treated with an agent for a disorder associated with aberrant ENDOX expression or activity in which a test sample is obtained and ENDOX protein or nucleic acid is detected (e.g., wherein the presence of ENDOX protein or nucleic acid is diagnostic for a subject that can be administered the agent to treat a disorder associated with aberrant ENDOX expression or activity).

The methods of the invention can also be used to detect genetic lesions in an ENDOX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an ENDOX-protein, or the misexpression of the ENDOX gene. For example, such genetic lesions can be detected by ascertaining the existence of at least one of: (i) a deletion of one or more nucleotides from an ENDOX gene; (ii) an addition of one or more nucleotides to an ENDOX gene; (iii) a substitution of one or more nucleotides of an ENDOX gene, (iv) a chromosomal rearrangement of an ENDOX gene; (v) an alteration in the level of a messenger RNA transcript of an ENDOX gene, (vi) aberrant modification of an ENDOX gene, such as of the methylation pattern of the genomic DNA, (vii) the presence of a non-wild-type splicing pattern of a messenger RNA transcript of an ENDOX gene, (viii) a non-wild-type level of an ENDOX protein, (ix) allelic loss of an ENDOX gene, and (x) inappropriate post-translational modification of an ENDOX protein. As described herein, there are a large number of assay techniques known in the art which can be used for detecting lesions in an ENDOX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the ENDOX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an ENDOX gene under conditions such that hybridization and amplification of the ENDOX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an ENDOX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in ENDOX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in ENDOX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the ENDOX gene and detect mutations by comparing the sequence of the sample ENDOX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the ENDOX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type ENDOX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in ENDOX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an ENDOX sequence, e.g., a wild-type ENDOX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in ENDOX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control ENDOX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an ENDOX gene.

Furthermore, any cell type or tissue, preferably peripheral blood leukocytes, in which ENDOX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on ENDOX activity (e.g., ENDOX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of ENDOX protein, expression of ENDOX nucleic acid, or mutation content of ENDOX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of ENDOX protein, expression of ENDOX nucleic acid, or mutation content of ENDOX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an ENDOX modulator, such as a modulator identified by one of the exemplary screening assays described herein.

Monitoring of Effects During Clinical Trials

Monitoring the influence of agents (e.g., drugs, compounds) on the expression or activity of ENDOX (e.g., the ability to modulate aberrant cell proliferation and/or differentiation) can be applied not only in basic drug screening, but also in clinical trials. For example, the effectiveness of an agent determined by a screening assay as described herein to increase ENDOX gene expression, protein levels, or upregulate ENDOX activity, can be monitored in clinical trails of subjects exhibiting decreased ENDOX gene expression, protein levels, or downregulated ENDOX activity. Alternatively, the effectiveness of an agent determined by a screening assay to decrease ENDOX gene expression, protein levels, or downregulate ENDOX activity, can be monitored in clinical trails of subjects exhibiting increased ENDOX gene expression, protein levels, or upregulated ENDOX activity. In such clinical trials, the expression or activity of ENDOX and, preferably, other genes that have been implicated in, for example, a cellular proliferation or immune disorder can be used as a “read out” or markers of the immune responsiveness of a particular cell.

By way of example, and not of limitation, genes, including ENDOX, that are modulated in cells by treatment with an agent (e.g., compound, drug or small molecule) that modulates ENDOX activity (e.g., identified in a screening assay as described herein) can be identified. Thus, to study the effect of agents on cellular proliferation disorders, for example, in a clinical trial, cells can be isolated and RNA prepared and analyzed for the levels of expression of ENDOX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of ENDOX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an ENDOX protein, mRNA, or genomic DNA in the preadministration sample; (iii) obtaining one or more post-administration samples from the subject; (iv) detecting the level of expression or activity of the ENDOX protein, mRNA, or genomic DNA in the post-administration samples; (v) comparing the level of expression or activity of the ENDOX protein, mRNA, or genomic DNA in the pre-administration sample with the ENDOX protein, mRNA, or genomic DNA in the post administration sample or samples; and (vi) altering the administration of the agent to the subject accordingly. For example, increased administration of the agent may be desirable to increase the expression or activity of ENDOX to higher levels than detected, i.e., to increase the effectiveness of the agent. Alternatively, decreased administration of the agent may be desirable to decrease expression or activity of ENDOX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant ENDOX expression or activity. The disorders include metabolic disorders, diabetes, obesity, infectious disease, anorexia, cancer-associated cachexia, and the various dyslipidemias, metabolic disturbances associated with obesity, the metabolic syndrome X and wasting disorders associated with chronic diseases and various cancers. These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endoggenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant ENDOX expression or activity, by administering to the subject an agent that modulates ENDOX expression or at least one ENDOX activity. Subjects at risk for a disease that is caused or contributed to by aberrant ENDOX expression or activity can be identified by, for example, any or a combination of diagnostic or prognostic assays as described herein. Administration of a prophylactic agent can occur prior to the manifestation of symptoms characteristic of the ENDOX aberrancy, such that a disease or disorder is prevented or, alternatively, delayed in its progression. Depending upon the type of ENDOX aberrancy, for example, an ENDOX agonist or ENDOX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating ENDOX expression or activity for therapeutic purposes. The modulatory method of the invention involves contacting a cell with an agent that modulates one or more of the activities of ENDOX protein activity associated with the cell. An agent that modulates ENDOX protein activity can be an agent as described herein, such as a nucleic acid or a protein, a naturally-occurring cognate ligand of an ENDOX protein, a peptide, an ENDOX peptidomimetic, or other small molecule. In one embodiment, the agent stimulates one or more ENDOX protein activity. Examples of such stimulatory agents include active ENDOX protein and a nucleic acid molecule encoding ENDOX that has been introduced into the cell. In another embodiment, the agent inhibits one or more ENDOX protein activity. Examples of such inhibitory agents include antisense ENDOX nucleic acid molecules and anti-ENDOX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an ENDOX protein or nucleic acid molecule. In one embodiment, the method involves administering an agent (e.g., an agent identified by a screening assay described herein), or combination of agents that modulates (e.g., up-regulates or down-regulates) ENDOX expression or activity. In another embodiment, the method involves administering an ENDOX protein or nucleic acid molecule as therapy to compensate for reduced or aberrant ENDOX expression or activity.

Stimulation of ENDOX activity is desirable in situations in which ENDOX is abnormally downregulated and/or in which increased ENDOX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The ENDOX nucleic acids and proteins of the invention are useful in potential prophylactic and therapeutic applications implicated in a variety of disorders including, but not limited to: metabolic disorders, diabetes, obesity, infectious disease, anorexia, cancer-associated cachexia, and the various dyslipidemias, metabolic disturbances associated with obesity, the metabolic syndrome X and wasting disorders associated with chronic diseases and various cancers.

As an example, a cDNA encoding the ENDOX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, and the various dyslipidemias.

Both the novel nucleic acid encoding the ENDOX protein, and the ENDOX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies which immunospecifically-bind to the novel substances of the invention for use in therapeutic or diagnostic methods.

### EXAMPLES

The following examples illustrate by way of non-limiting example various aspects of the invention.

A family of new human genes, related to the gene encoding human acyl-CoA binding protein (ACBP)/Diazepam-binding Inhibitor (DBI), was identified by an analysis of expressed sequences and genomic DNA sequences. See example 6. The human family consists of 7 novel and 3 known ENDO genes that all contain a highly conserved domain of 20 amino acids. ACBP/DBI is processed to produce a biologically active 18 amino acid peptide (ODN) that influences organismal energy metabolism. It is expected that biological processing of the other family members would lead to other metabolism-regulating peptides.

Synthetic peptides derived from the conserved domain of each human family member, were used in studies of metabolism and to generate polyclonal antibodies in rabbits. The synthetic peptides from the conserved domain of each human and a rat family member were injected into various mouse strains. The injected peptides elicited distinct changes in organismal energy metabolism that effect adipose stores, muscle mass, insulin secretion, glucose utilization and serum lipid levels (triglycerides and cholesterol). See example 1.

Consensus sequences can be derived from the full-length proteins and individual peptides having specific metabolic effects. See example 2 and 3. These human peptides, peptides from non-human species, mutant peptides derived by rational or combinatorial changes based upon the consensus sequences, antibodies and small molecule drugs that interact with the full length proteins, peptides, processing enzymes and/or receptors could have important therapeutic value in the treatment of metabolic disorders. See example 1 and 4. These disorders include anorexia, cancer-associated cachexia, obesity, Type I and Type II diabetes and the various dyslipidemias.

The invention is also useful as a marker for various cancer types. See example 5

### Example 1

### Peptide-Elicited Metabolic Effects in AKR Mice

A study was performed to determine the effect of daily ip doses (14 days) of the metabolic regulating peptides (MRPs) derived from the human and rat endozepines on metabolic parameters in AKR (obesity and diabetes prone) and C57B1/6 (control) mice. This example summarizes the effects of the peptides on AKR mice. Serum cholesterol, triglycerides, insulin and glucose were monitored along with body weight, body weight change, relative organ weights of liver, reproductive caudal abdominal fat pads, pancreatic and mesenteric tissue weights, quadriceps muscle weight, food and water intake. Histopathologic analysis was performed on selected tissue samples.

Peptides were injected into both AKR and C57B1/6 mice. The AKR mice are heavier and have higher serum glucose levels (particularly in response to manipulation) than control C57B1/6 mice. As such, the AKR mice serve as a polygenic model for the forms of obesity and type II diabetes prevalent in the human population. Table 29 summarizes the metabolic effects elicited by each MRP in the AKR mice. MRPs and a peptide with a sequence randomized from the amino acids found in ODN were given by intraperitoneal injection at the indicated dose in 1% DMSO. Control animals were either not manipulated, injected with 1% DMSO or injected with phosphate-buffered saline (PBS). The data in the Table 29 is recorded separately for males and females where indicated and combined for both sexes otherwise. The change from baseline (pre-injection) on serum glucose is reported in mg/dl. The insulin is in microU/ml, the “relative” measures of mesenteric (males and females) fat, uterine fat (females) and quadriceps muscle are recorded as a percent of body weight. The other findings represent consistent findings for both males and females. Statistically significant findings (p<0.05) are shaded red (or dark gray) for increases (increased glucose for MRP1₁₈, MRP2₂₀, MRP3₁₈, MRP4₁₈, MRP5₂₀, MRP720, and MRP10₂₀; and increased relative muscle for MRP1₂₀ and MRP10₂₀) and blue-green (or light gray) for decreases (decreased glucose for MRP1₂₀, MRP4₂₀ and MRP6₂₀; decreased ending insulin for MRP1₂₀, MRP1₁₈, MRP2₂₀, MRP3₁₈, MRP4₁₈, MRP8₂₀, MRP10₂₀ and MRP1₁₈ (Rat); decreased relative fat ratio for MRP1₂₀, MRP5₂₀, MRP10₂₀ and MRP1₁₈ (Rat); and decreased relative muscle for MRP2₂₀ and MRP7₂₀).

A separate test group was established for each peptide examined, a vehicle control and a non-manipulated group. Each test group consisted of five male and five female AKR mice (10 per peptide). The mice were acclimated for 2 days following shipment. The mice were weighed and divided into groups. Food weight and water volume per cage was measured and recorded.

The mice acclimated another 5 days. On day (−2) pre-injection glucose measurements were made utilizing a glucometer (Johnson & Johnson; One touch Sure Step). Blood samples were obtained by, first, heating the mice under a heat lamp for approximately 5 minutes before obtaining a drop of blood via the tail vein. Dosing of the various peptides began on day (0). Peptides were prepared identically and dosed at the same concentration (0.05 mg/ml in PBS with 1%DMSO, with a dose volume of 10 ml/kg) except as noted in Table 29. Stock peptide solutions were prepared by dissolving 2.5 mg of peptide in 0.5 ml of DMSO with 1-2 minutes of sonication, if required, followed by the addition of 0.5 ml of sterile PBS to each vial. Dosing solutions were prepared weekly by the addition of 0.5 ml of the peptide stock solution to 24.5 mls of sterile PBS. All dose solutions were kept refrigerated throughout the entire study.

The mice were weighed and given daily ip injections of the various peptides or vehicle for 14 consecutive days. Every seven days food weight and water volume was measured and recorded per cage. Blood glucose measurements (via glucometer) were also taken and recorded every 7 days. Blood glucose measurements were taken between 8:00 and 10:00 am and the animals were dosed between 9:00 and 11:00 am daily.

On day 14, one hour prior to necropsy and blood collection, the mice were injected with the final dose of the various peptides or vehicle. Blood (heparinized plasma) was obtained via cardiac or orbital puncture. Plasma chemistries, glucose and insulin, as well as a final glucometer reading were taken at the same time to calibrate/corroborate the glucometer readings. Animals were dissected and placed in formalin for histological sample preparation. Mesenteric fat and pancreas, liver, quadriceps muscle, and caudal abdominal fat pads were removed and weighed for each animal. This study illustrates that the ENDOX peptides can be used to modulate various metabolic functions and treat metabolic disorders.

TABLE 29

| | Dose | | Ending | Relative Fat | Relative | |
|---|---|---|---|---|---|---|
| Peptide | (mg/kg) | Δ Glucose | Insulin | (Mesenteric/ | Muscle | Other |
| Name | (vehicle | (M/F) | (M/F) | Uterine) | (M/F) | Findings |
| MRP1₂₀ | 0.5 | −23.0 | 1.78 ± 0.24 | 2.02 ± 0.08 | 0.83 ± 0.02 | |
| | DMSO | (−15%) | 1.96 ± 0.12 | 1.04 ± 0.18 | 0.86 ± 0.07 | |
| MRP1₁₈ | 0.5 | +66.1 | 1.08 ± 0.21 | 1.76 ± 0.08 | 0.72 ± 0.02 | ↑cholesterol |
| (ODN) | DMSO | (+56%) | 0.63 ± 0.13 | 1.44 ± 0.21 | 0.65 ± 0.02 | ↑triglyceride |
| MRP2₂₀ | 0.5 | +78.4 | 2.20 ± 0.30 | 1.72 ± 0.15 | 0.62 ± 0.03 | ↑cholesterol |
| | DMSO | (+58%) | 1.90 ± 0.30 | 1.24 ± 0.25 | 0.60 ± 0.04 | ↑triglyceride |
| MRP3₂₀ | 0.5 | −3.6 | 3.70 ± 0.32 | 1.95 ± 0.10 | 0.73 ± 0.03 | ↓Liver |
| | PBS | (−3%) | 4.28 ± 0.98 | 1.74 ± 0.27 | 0.63 ± 0.02 | |
| | | +32.6 | | | | |
| MRP3₁₈ | 0.5 | +70.8 | ND | 2.01 ± 0.04 | 0.66 ± 0.03 | |
| | DMSO | (+58%) | 1.52 ± 0.43 | 1.53 ± 0.22 | 0.63 ± 0.02 | |
| MRP4₂₀ | 0.1 | −36.4 | 3.33 ± 0.33 | 2.00 ± 0.16 | 0.76 ± 0.04 | |
| | DMSO | (−23%) | 2.40 ± 0.17 | 1.56 ± 0.22 | 0.61 ± 0.05 | |
| MRP4₁₈ | 0.5 | +62.1 | 0.58 ± 0.08 | 1.73 ± 0.09 | 0.68 ± 0.04 | ↑cholesterol |
| | DMSO | (+51%) | 0.78 ± 0.18 | 1.16 ± 0.18 | 0.63 ± 0.02 | ↓Liver ↑CPK |
| MRP5₂₀ | 0.5 | +72.0 | 2.46 ± 0.32 | 1.63 ± 0.26 | 0.77 ± 0.07 | |
| | DMSO | (+59%) | 2.40 ± 0.43 | 1.20 ± 0.26 | 0.64 ± 0.03 | |
| MRP6₂₀ | 0.5 | −52.0 | 2.95 ± 0.35 | 2.04 ± 0.12 | 0.79 ± 0.07 | |
| | DMSO | (−32%) | 2.70 ± 0.80 | 1.42 ± 0.18 | 0.74 ± 0.05 | |
| MRP7₂₀ | 0.5 | +83.8 | 2.35 ± 0.44 | 1.70 ± 0.08 | 0.67 ± 0.08 | ↓cholesterol |
| | DMSO | (+64%) | 1.88 ± 0.51 | 1.47 ± 0.36 | 0.52 ± 0.08 | ↓triglyceride |
| | | | | | | ↑CPK ↓Liver |
| MRP8₂₀ | 0.5 | +30.6 | 1.15 ± 0.36 | 1.66 ± 0.25 | 0.74 ± 0.06 | ↓cholesterol |
| | DMSO | (+26%) | 1.28 ± 0.78 | 1.99 ± 0.45 | 0.62 ± 0.02 | ↓triglyceride |
| MRP9₂₀ | 0.5 | +28.6 | 4.08 ± 1.72 | 1.74 ± 0.09 | 0.70 ± 0.03 | ↓cholesterol |
| | DMSO | (+22%) | 2.60 ± 0.44 | 1.24 ± 0.14 | 0.67 ± 0.04 | |
| MRP10₂₀ | 0.5 | +61.4 | 0.82 ± 0.33 | 1.12 ± 0.20 | 0.81 ± 0.03 | ↑↑CPK |
| | DMSO | (+56%) | 0.53 ± 0.03 | 1.85 ± 0.36 | 0.70 ± 0.05 | |
| MRP1₁₈ | 0.5 | +7.8 | 2.16 ± 0.26 | 1.93 ± 0.16 | 0.79 ± 0.06 | |
| | DMSO | (+6%) | 1.98 ± 0.19 | 1.37 ± 0.24 | 0.71 ± 0.04 | |
| | | +17.2 | | | | |
| Rando | 0.5 | +49.4 | 1.42 ± 0.52 | 1.70 ± 0.08 | 0.68 ± 0.03 | |
| | DMSO | (+40%) | 0.62 ± 0.12 | 1.70 ± 0.09 | 0.67 ± 0.03 | |
| Vehic | PBS | +8.8 | 3.92 ± 0.35 | 1.89 ± 0.15 | 0.70 ± 0.04 | |
| | | (+6%) | 3.58 ± 0.22 | 2.50 ± 0.32 | 0.78 ± 0.05 | |
| | | +25.1 | | | | |
| Vehic | DMSO | +40.2 | 2.72 ± 0.62 | 1.77 ± 0.08 | 0.73 ± 0.03 | |
| | | (+30%) | 0.74 ± 0.20 | 1.31 ± 0.15 | 0.63 ± 0.04 | |

### Example 2

### Clustal W Alignments

Table 30 shows the Clustal W alignment of all 10 human endozepines. The family consists of two classes of polypeptides—7 short polypeptides of about 90 amino acids and 3 longer polypeptides containing regions of homology to the other family members at their N termini. There is no homology between the longer forms at their C-termini. This alignment and the phylogenetic distances suggest that the family may have evolved by gene duplications, fusions and independent evolution.

[image]

[image]

Table 30-1 lists the sequence identifiers and sequence identification numbers (SEQ ID NO) for the sequences displayed in Table 30.

TABLE 30-1

| SEQUENCE IDENTIFIER | SEQ ID NO |
|---|---|
| Endo1_MRP-6 | SEQ ID NO: 98 |
| Endo8_MRP-1_ACBP_{—} | SEQ ID NO: 99 |
| Endo7_MRP-9 | SEQ ID NO: 100 |
| Endo3_MRP-3 | SEQ ID NO: 101 |
| Endo4_MRP-4 | SEQ ID NO: 102 |
| Endo2_MRP-5 | SEQ ID NO: 103 |
| Endo5_MRP-7 | SEQ ID NO: 104 |
| Endo9_MRP-2_PECI_{—} | SEQ ID NO: 106 |
| Endo10_MRP-10_ELP_{—} | SEQ ID NO: 105 |
| Endo6_MRP-8 | SEQ ID NO: 107 |

### Example 3

### Consensus Sequences for the Metabolism—Regulating Peptides

All of the MRPs and several subsets of peptides, that have similar metabolic effects in AKR mice were identified in Table 29. These can be aligned to identify consensus sequences which are identified in Table 31. The general and preferred consensus sequences are deduced by inspection. The most conserved amino acid at each position in the 20 amino acid motif is in bold print. If that amino acid is invariant in a particular metabolic subset then it is also underlined. If the amino acid at a particular position is highly variable it is represented by an “X.” When a small number of amino acids are found at a particular position then they are enclosed within [brackets]. The “general” consensus allows for the most variability in a peptide sequence that could have similar metabolic effects. The “preferred” consensus sequence is limited to the amino acids found in the inspected peptide subset. Particular subsets of peptides were identified that are associated with cholesterol-lowering properties, fat mass reducing, insulin-lowering, glucose lowering, glucose-raising and muscle mass building properties.

Table 30-1 lists the sequence identifiers and sequence identification numbers (SEQ ID NO) for the sequences displayed in Table 31.

[image]

[image]

### Example 4

### Effects on Mesenteric Adipocyte Size

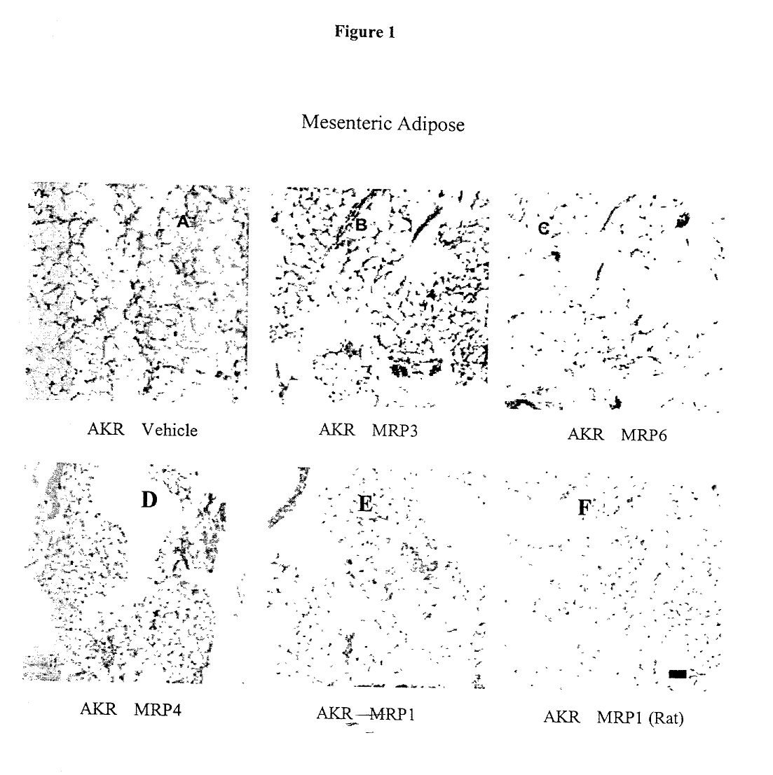

FIG. 1 consists of 6 photomicrographs of mesenteric adipose fixed from male AKR mice after treatment with the agent indicated beneath each panel. In the top panels (A-C) large adipocytes predominate whereas in the bottom panels (D-F) smaller adipocytes predominate. This correlates with the decrease in the mass of that particular adipose deposit in response to the specific treatment. There are subsets of MRPs that affect the mass of specific adipose deposits and subsets of MRPs that have influence on other metabolic parameters.

### Example 5

### Gene Expression, Cloning & Antibody Production Summary

Table 32 lists the members of the endozepine family (EndoX) and the corresponding bio-active Metabolism-Regulating Peptide (MRP-#) synthesized from each endozepine. In addition, the tissue(s) where the gene is most highly expressed is indicated from the result of real-time quantitative RTQ-PCR (TaqMan) using total RNA as well as by traditional PCR using ds cDNA as a template. PCR products corresponding to the coding regions of the various endozepines have been amplified for cloning purposes as noted. Anti-peptide antibodies have been generated in rabbits for each human synthetic 20 amino acid peptide. Anti-peptide titers that have been determined (ELISA) are indicated.

TABLE 32

| | | | Gene Expression Distribution | | | | |
|---|---|---|---|---|---|---|---|
| | | | | | Traditional RT-PCR | | |
| | | | | | | Physical | |
| | | | | | | Product | |
| | Bio-Active | | TaqMan - Principal Tissues | | | For | Polyclonal Antisera |
| Endozepine | Peptide | Bio-Active Peptide Sequence | | Cancer | Normal Tissue | Cloning | Production |
| Name | Name | 20-mers | Normal Tissues | Tissues | Distribution | or Cloned | Highest Titer |
| Endo1 | MRP-6 | QATQGDCDIPGPPASDVRAR | Liver | Multiple CAs | Brain | Yes | Antibody Available |
| | | (SEQ ID NO: 117) | Endothelial cells | | Heart | | |
| | | | | | Liver | | |
| | | | | | Pancreas | | |
| | | | | | Skeletal Muscle | | |
| | | | | | Small Intestine | | |
| Endo2 | MRP-5 | QAVIGNINIECSEMLELKGK | Brain | Colon CA | Adipose | Yes | Antibody Available |
| | | (SEQ ID NO: 110) | Pancreas | Lung CA | Bone Marrow | | |
| | | | | | Fetal Brain | | |
| | | | | | Fetal Liver | | |
| Endo3 | MRP-3 | RATVGNIKTERPGMVDFKGK | Adipose | Breast CA | Adipose | Yes | 2,000 |
| | | (SEQ ID NO: 109) | Skeletal Muscle | | Brain | | |
| | | | | | Liver | | |
| | | | | | Heart | | |
| | | | | | Pancreas | | |
| | | | | | Skeletal Muscle | | |
| | | | | | Small Intestine | | |
| Endo4 | MRP-4 | QAIVGDINIACPGMLDLKGK | Hematopoietic | Low | Liver | Yes | Antibody Available |
| | | (SEQ ID NO: 112 | | Expression | | | |
| Endo5 | MRP-7 | QAIIGDINIEYLGMLDFKGK | | | | | Antibody Available |
| | | (SEQ ID NO: 111) | | | | | |
| Endo6 | MRP-8 | QATEGPCKLSRPGFWDPIGR | Skeletal Muscle | Multiple CAs | Brain | Yes | 13,000 |
| | | (SEQ ID NO: 116) | | | Liver | | |
| | | | | | Muscle | | |
| Endo7 | MRP-9 | QATVHDLNTEWPRMLDLKGK | Adipose | Liver CA | Adip. | Yes | 12,000 |
| | | (SEQ ID NO: 113) | | | Fetal Brain | | |
| | | | | | Fetal Liver | | |
| Endo8 | MRP-1 | QATVGDINTERPGMLDFTGK | Heart | Breast CA | Brain | Yes | 14,000 |
| (ACBP/DBI/ | | (SEQ ID NO: 108) | Skeletal Muscle | Colon CA | Liver | | |
| CCK-RP) | | | Liver | Melanoma | | | |
| | | | Endothelial cells | | | | |
| Endo9 | MRP-2 | QATEGPCNMPKPGVFDLINK | | | Brain | Yes | 3,600 |
| (PECI) | | (SEQ ID NO: 115) | | | Liver | | |
| (D2,D3 | | | | | Skeletal Muscle | | |
| Enoyl CoA | | | | | | | |
| Isomerase) | | | | | | | |
| Endo10 | MRP-10 | QVKVGNCNTPKPSFFDFEGK | | | Fetal Brain | Yes | 10,000 |
| (ELP) | | (SEQ ID NO: 114) | | | Fetal Liver | | |
| | | | | | Bone Marrow | | |
| | | 18-mers | | | | | |
| Endo8 | MRP-1s | QATVGDINTERPGMLDFT-- | | | | | |
| (ACBP/DBI/ | (ODN) | (SEQ ID NO: 118) | | | | | |
| CCK-RP) | | | | | | | |
| Rat ODN | MRP-Rn. | QATVGDVNTDRPGLLDLK-- | | | | | |
| | ODN | (SEQ ID NO: 119) | | | | | |
| Endo3 | MRP-3s | RATVGNIKTERPGMVDFK-- | | | | | |
| | | (SEQ ID NO: 120) | | | | | |

### Example 6

### Phylogenetic Relationships Among the Novel and Known Endozepines

Table 33 indicates the relatedness of the various full-length endozepines as well as the MRPs encoded within each endozepine. The distances are computed by the program “Phylip” that calculates neighbor-joining distances, a method for describing the relative relatedness of the input sequences.

[image]

### Example 7

### Identification of Related Peptides in Other Proteins

Tables 34 and 35 illustrate how the specific consensus motifs can be used to identify polypeptides in other animal species that may be involved in the regulation of the same metabolic parameter. The GenBank database was searched using two general consensus sequences, the first associated with muscle mass building (Table 34) and the second associated with fat mass reduction (Table 35). The consensus sequences identified in each peptide are shown in bold font. Many polypeptides from multiple species were identified with 0, 1 or 2 mismatches from the consensus sequence. The highest similarity is seen in ACBPs from other species. This is also seen in FIG. 1, which illustrates that orthologous sequences can be found for 6 of the 10 members of the human endozepine family.

TABLE 34
Muscle-Raising Motif

Pattern Submitted: ‘qxxvgxxntx[kr]pxxxdffxgk’ (SEQ ID NO:46)
Database Analyzed: Non-Redundant Composite *
Matching Sequences To Show: Top 50
Mismatches Allowed: 3
Allow Insertions/Deletions: no
Allow matches to database ambiguities: no
Database contains 553,883 sequences.
Reporting all 29 matching sequences (did not trigger cutoff of 50 matches).
There are 4 equivalence classes of equally good matches.
SWISSPROT-ACC:P07107 ACYL-COA-BINDING PROTEIN (ACBP) (DIAZEPAM BINDING
INHIBITOR) (DBI) (ENDOZEPINE) (EP)—Bos taurus (Bovine), 86 aa.

| | | | |
|---|---|---|---|
| # Mismatches | Match Position | Match Context | |
| 0 | 33-52 | IYSHYKQATVGDINTERPGMLDFKGKAKWDAW | (SEQ ID NO:121) |

SWISSPROT-ACC:P07108 ACYL-COA-BINDING PROTEIN (ACBP) (DIAZEPAM BINDING
INHIBITOR) (DBI) (ENDOZEPINE) (EP)—Homo sapiens (Human), 86 aa.

| | | | |
|---|---|---|---|
| # Mismatches | Match Position | Match Context | |
| 0 | 33-52 | IYGHYKQATVGDINTERPQMLDFTGKAKWDAW | (SEQ ID NO:122) |

SPTREMBL-ACC:Q9VLS4 CG8498 PROTEIN—Drosophila melanogaster (Fruit fly), 90
aa.

| | | | |
|---|---|---|---|
| # Mismatches | Match Position | Match Context | |
| 0 | 35-54 | LYSLYKQATVGDCNTDKPGFLDFKGKAKWEAW | (SEQ ID NO:123) |

SPTREMBL-ACC:Q9PRL8 ACYL-COENZYME A BINDING PROTEIN, ACBP—Gallus gallus
(Chicken), 86 aa.

| | | | |
|---|---|---|---|
| # Mismatches | Match Position | Match Context | |
| 0 | 33-52 | VYSHYKQATVGDVNTDRPGMLDFKGKAKWDAW | (SEQ ID NO:124) |

REMTREMBL-ACC:CAA44618 ACYL-COA-BINDING PROTEIN /DIAZEPAM-BINDING INHIBITOR
—synthetic construct, 87aa

| | | | |
|---|---|---|---|
| # Mismatches | Match Position | Match Context | |
| 0 | 34-53 | IYSHYKQATVGDINTERPGMLDFKGKAKWDAW | (SEQ ID NO:125) |

pir-id:NZHU endozepine [validated]—human, 87 aa.

| | | | |
|---|---|---|---|
| # Mismatches | Match Position | Match Context | |
| 0 | 34-53 | IYGHYKQATVGDINTERPGMLDFTGKAKWDAW | (SEQ ID NO:126) |

pir-id:S63593 acyl-coenzyme A-binding protein—turtle, 86 aa.

| | | | |
|---|---|---|---|
| # Mismatches | Match Position | Match Context | |
| 0 | 33-52 | IYSHFKQATVGDINTERPGFLDFKGKAKWDAW | (SEQ ID NO:127) |

pir-id:S63594 acyl-coenzyme A-binding protein—mallard, 86 aa.

| | | | |
|---|---|---|---|
| # Mismatches | Match Position | Match Context | |
| 0 | 33-52 | VYSHYKQATVGDVNTDRPGMLDFKGKAKWDAW | (SEQ ID NO:128) |

P31786 ACYL-COA-BINDING PROTEIN (ACBP) (DIAZEPAM BINDING INHIBITOR) (DBI)
(ENDOZEPINE) (EP)—Mus musculus (Mouse), 86 aa.

| | | | |
|---|---|---|---|
| # Mismatches | Match Position | Match Context | |
| 1 | 33-52 | IYSHFKQATVGDVNTDRPGLLDLKGKAKWDSW | (SEQ ID NO:129) |

SWISSPROT-ACC:P12026 ACYL-COA-BINDING PROTEIN (ACBP) (DIAZEPAM BINDING
INHIBITOR) (DBI) (ENDOZEPINE) (EP) [CONTAINS:DBI(32-86)]—Sus scrofa
(Pig), 86 aa.

| | | | |
|---|---|---|---|
| # Mismatches | Match Position | Match Context | |
| 1 | 33-52 | IYSHYKQATVGDINTERPGILDLKGKAKWDAW | (SEQ ID NO:130) |

TABLE 35

Identification of Related Peptides In Other Proteins
Adipose-Lowering Motif

Pattern Submitted: ‘qax[vi]gnin[ti]expxml[de]fxgk’ (SEQ ID NO:40)
Database Analyzed: Non-Redundant Composite *
Matching Sequences To Show: Top 50
Mismatches Allowed: 3
Allow Insertions/Deletions: no
Allow matches to database ambiguities: no
Database contains 553,883 sequences.
Reporting all 14 matching sequences (did not trigger cutoff of 50
matches)
There are 3 equivalence classes of equally good matches.
SWISSPROT-ACC:P07107 ACYL-COA-BINDING PROTEIN (ACBP) (DIAZEPAM BINDING
INHIBITOR) (DBI) (ENDOZEPINE) (HP)—Bos taurus (Bovine), 86 aa.

| | | | |
|---|---|---|---|
| # Mismatches | Match Position | Match Context | |
| 1 | 33-52 | IYSHYKQATVGDINTERPGMLDFKGKAKWDAW | SEQ ID NO:131) |

SWISSPROT-ACC:P07108 ACYL-COABINDING PROTEIN (ACBP) (DIAZEPAM BINDING
INHIBITOR) (DBI) (ENDOZEPINE) (EP)—Homo sapiens (Human), 86 aa.

| | | | |
|---|---|---|---|
| # Mismatches | Match Position | Match Context | |
| 1 | 33-52 | IYGHYKQATVGDINTERPGMLDFTGKAKWDAW | (SEQ ID NO:132) |

REMTREMBL-ACC:CAA44618 ACYL-COA-BINDING PROTEIN /DIAZEPAM-BINDING INHIBITOR
—synthetic construct,87aaaa.

| | | | |
|---|---|---|---|
| # Mismatches | Match Position | Match Context | |
| 1 | 4-53 | IYSHYKQATVGDINTERPGMLDFKGKAKWDAW | (SEQ ID NO:133) |

pir-id:NZHU endozepine [validated]—human, 87 aa.

| | | | |
|---|---|---|---|
| ™Mismatches | Match Position | Match Context | |
| 1 | 34-53 | IYGHYKQATVGDINTERPGMLDFTGKAKWDAW | (SEQ ID NO:134) |

SWISSPROT-ACC:P45882 ACYL-COA-BINDING PROTEIN (ACBP) (DIAZEPAM BINDING
INHIBITOR) (DBI) (ENDOZEPINE) (EP)—Anas platyrhynchos (Domestic duck), 103
aa.

| | | | |
|---|---|---|---|
| # Mismatches | Match Position | Match Context | |
| 2 | 50-69 | LYGFYKQATVGDINIECPGMLDLKGKAKWEAW | (SEQ ID NO:135) |

pir-id:S63593 acyl-coenzyme A-binding protein—turtle, 86 aa.

| | | | |
|---|---|---|---|
| # Mismatches | Match Position | Match Context | |
| 2 | 33-52 | IYSHFKQATVGDINTERPGFLDFKGKAKWDAW | (SEQ ID NO:136)+TZ,1/47 |

### Example 8

### Orthologous Gene Products in Multiple Species

Table 36 illustrates the high degree of conservation of the MRP motifs across diverse species. The human MRP sequences were used to search GenBank for related sequences. Eleven other sequences with a high degree of similarity were identified. Using a larger peptide centered on the 20 amino acid MRP domain will identify additional polypeptides. The 11 entries from other species are represented by their accession numbers. The indicated set of 21 peptides from species ranging from frogs to humans was examined for their relationship to the set of 10 human peptides by the PHYLIP, PILEUP and Clustal W algorithms. As can be seen in Table 36, there are non-human peptides that are more like some human sequences than other human sequences. This suggests that the peptide-containing genes arose and followed divergent paths in energy metabolism early in evolution.

[image]

Table 37 also illustrates the high degree of conservation of the MRP motifs across diverse species.

[image]

Table 37-1 lists the sequence identifiers and sequence identification numbers (SEQ ID NO) for the sequences displayed in Table 37.

TABLE 37-1

| SEQUENCE IDENTIFIER | SEQ ID NO |
|---|---|
| U09205 | SEQ ID NO: 137 |
| Endo4-MRP4 | SEQ ID NO: 112 |
| Endo2-MRP5 | SEQ ID NO: 110 |
| Endo5-MRP7 | SEQ ID NO: 111 |
| U04823 | SEQ ID NO: 138 |
| X75596 | SEQ ID NO: 139 |
| AL096874 | SEQ ID NO: 140 |
| M15886 | SEQ ID NO: 141 |
| Endo8-MRP1 | SEQ ID NO: 108 |
| X61431 | SEQ ID NO: 142 |
| M20268 | SEQ ID NO: 143 |
| AB019792 | SEQ ID NO: 144 |
| Endo3-MRP3 | SEQ ID NO: 109 |
| Endo7-MRP9 | SEQ ID NO: 113 |
| M15888 | SEQ ID NO: 145 |
| Endo6-MRP8 | SEQ ID NO: 116 |
| AF006493 | SEQ ID NO: 146 |
| AF153613 | SEQ ID NO: 147 |
| Endo9-MRP2 | SEQ ID NO: 115 |
| Endo10-MRP10 | SEQ ID NO: 114 |
| Endo1-MRP6 | SEQ ID NO: 117 |

### Example 9

### PCR Products of Endozepine Coding Sequences

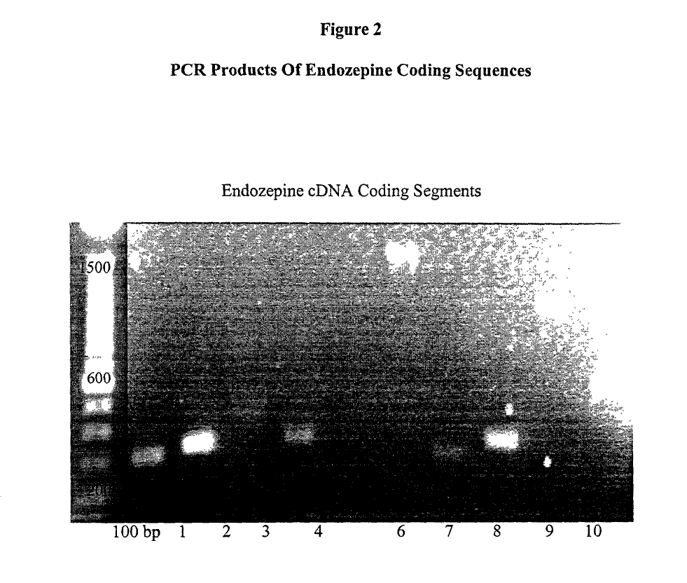

The coding sequence of each human endozepine was cloned into numerous expression vectors in order to examine the complex ways in which these secreted proteins and their biologically active MRPs affect energy metabolism. Some of the cDNAs have been cloned in bacteria in order to prepare recombinant proteins. The complete set of proteins were expressed in bacteria, yeast and mammalian cells to study their individual biochemical activities, protein-protein interactions and how they modulate energy metabolism in isolated cells or whole animals. Physical DNA molecules for this purpose were generated by PCR from the various tissues, noted above in Table 29, and can be seen on this ethidium bromide-stained agarose gel (FIG. 2). The individual PCR products for Endos 1-4, and Endos 6-10 are of the expected size as deduced from the sequences (including 42 bp of flanking cloning sequence here).

### Example 10

### Identification of ENDOX Clones

The novel ENDOX target sequences identified in the present invention were subjected to the exon linking process to confirm the sequence. PCR primers were designed by starting at the most upstream sequence available, for the forward primer, and at the most downstream sequence available for the reverse primer. Table 9 shows the sequences of the PCR primers used for obtaining different clones. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

TABLE 50

PCR Primers for Exon Linking

| | | SEQ | | SEQ |
|---|---|---|---|---|
| ENDOX | | ID | | ID |
| Clone | primer 1(5′-3′) | NO | Primer 2 (5′-3′) | NO |
| 2A | ATAAGACATACAGAAGGAATGCCTGGA | 199 | TATAAGACATACAGAAGGAATGCCTGG | 200 |
| 4A | GGTGGTAAATGCTCCTTTTGTTTGTTT | 201 | ACATCAAGTTAACAGTATGCCTCTCCC | 202 |

### Example 11

### SNPs and cSNPs:

One or more consensus positions (Cons. Pos.) of the nucleotide sequence have been identified as SNPs as shown in Table 2. “Depth” represents the number of clones covering the region of the SNP. The Putative Allele Frequency (Putative Allele Freq.) is the fraction of all the clones containing the SNP. A dash (“-”), when shown, means that a base is not present. The sign “>” means “is changed to”.

TABLE 51
ENDO4A (CG55148-02) SNP DATA

| Position | Depth | Change | Putative Allele Freq. |
|---|---|---|---|
| 97 | 21 | A > G | 0.095 |
| 128 | 21 | T > C | 0.095 |
| 166 | 21 | A > G | 0.095 |

Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An isolated polypeptide comprising an amino acid sequence selected from the group consisting of:
(a) a mature form of an amino acid sequence selected from the group consisting of SEQ ID NO:2, 4, 6, 8, 10, 15, 16, 17, 18, 19, 20, 21, 23, 24, 26, 27, 29, 30, 32, 33, 35 to 45 inclusive, 47, 49, 153, 157, 166, 175, 179, 183, 192, and 198;
(b) a variant of a mature form of an amino acid sequence selected from the group consisting of SEQ ID NO:2, 4, 6, 8, 10, 15, 16, 17, 18, 19, 20, 21, 23, 24, 26, 27, 29, 30, 32, 33, 35 to 45 inclusive, 47, 49, 153, 157, 166, 175, 179, 183, 192, and 198, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NO:2, 4, 6, 8, 10, 15, 16, 17, 18, 19, 20, 21, 23, 24, 26, 27, 29, 30, 32, 33, 35 to 45 inclusive, 47, 49, 153, 157, 166, 175, 179, 183, 192, and 198; and
(d) a variant of an amino acid sequence selected from the group consisting of SEQ ID NO:2, 4, 6, 8, 10, 15, 16, 17, 18, 19, 20, 21, 23, 24, 26, 27, 29, 30, 32, 33, 35 to 45 inclusive, 47, 49, 153, 157, 166, 175, 179, 183, 192, and 198, wherein one or more amino acid residues in said variant differs from the amino acid sequence of said mature form, provided that said variant differs in no more than 15% of amino acid residues from said amino acid sequence.

2 The polypeptide of claim 1, wherein said polypeptide comprises the amino acid sequence of a naturally-occurring allelic variant of an amino acid sequence selected from the group consisting of SEQ ID NO:2, 4, 6, 8, 10, 15, 16, 17, 18, 19, 20, 21, 23, 24, 26, 27, 29, 30, 32, 33, 35 to 45 inclusive, 47, 49, 153, 157, 166, 175, 179, 183, 192, and 198.

3. The polypeptide of claim 2, wherein said allelic variant comprises an amino acid sequence that is the translation of a nucleic acid sequence differing by a single nucleotide from a nucleic acid sequence selected from the group consisting of SEQ ID NO:1, 3, 5, 7, 9, 22, 25, 28, 31, 34, 46, 48, 152, 156, 165, 174, 178, 182, and 191.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NO:2, 4, 6, 8, 10, 15, 16, 17, 18, 19, 20, 21, 23, 24, 26, 27, 29, 30, 32, 33, 35 to 45 inclusive, 47, 49, 153, 157, 166, 175, 179, 183, 192, and 198;
(b) a variant of a mature form of an amino acid sequence selected from the group consisting of SEQ ID NO:2, 4, 6, 8, 10, 15, 16, 17, 18, 19, 20, 21, 23, 24, 26, 27, 29, 30, 32, 33, 35 to 45 inclusive, 47, 49, 153, 157, 166, 175, 179, 183, 192, and 198, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NO:2, 4, 6, 8, 10, 15, 16, 17, 18, 19, 20, 21, 23, 24, 26, 27, 29, 30, 32, 33, 35 to 45 inclusive, 47, 49, 153, 157, 166, 175, 179, 183, 192, and 198;
(d) a variant of an amino acid sequence selected from the group consisting of SEQ ID NO:2, 4, 6, 8, 10, 15, 16, 17, 18, 19, 20, 21, 23, 24, 26, 27, 29, 30, 32, 33, 35 to 45 inclusive, 47, 49, 153, 157, 166, 175, 179, 183, 192, and 198, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NO: 2, 4, 6, 8, 10, 15, 16, 17, 18, 19, 20, 21, 23, 24, 26, 27, 29, 30, 32, 33, 35 to 45 inclusive, 47, 49, 153, 157, 166, 175, 179, 183, 192, and 198, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NO:1, 3, 5, 7, 9, 22, 25, 28, 31, 34, 46, 48, 152, 156, 165, 174, 178, 182, and 191.

9. The nucleic acid molecule of claim 5, wherein said nucleic acid molecule comprises a nucleotide sequence selected from the group consisting of:
(a) a nucleotide sequence selected from the group consisting of SEQ ID NO:1, 3, 5, 7, 9, 22, 25, 28, 31, 34, 46, 48, 152, 156, 165, 174, 178, 182, and 191;
(b) a nucleotide sequence differing by one or more nucleotides from a nucleotide sequence selected from the group consisting of SEQ ID NO:1, 3, 5, 7, 9, 22, 25, 28, 31, 34, 46, 48, 152, 156, 165, 174, 178, 182, and 191, provided that no more than 20% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence chosen from the group consisting of SEQ ID NO:1, 3, 5, 7, 9, 22, 25, 28, 31, 34, 46, 48, 152, 156, 165, 174, 178, 182, and 191, or a complement of said nucleotide sequence.

11. The nucleic acid molecule of claim 5, wherein the nucleic acid molecule comprises a nucleotide sequence selected from the group consisting of:
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that binds immunospecifically to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide, thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule, thereby determining the presence or amount of the nucleic acid molecule in said sample.

20. The method of claim 19 wherein presence or amount of the nucleic acid molecule is used as a marker for cell or tissue type.

21. The method of claim 20 wherein the cell or tissue type is cancerous.

22. A method of identifying an agent that binds to a polypeptide of claim 1, the method comprising:
(a) contacting said polypeptide with said agent; and
(b) determining whether said agent binds to said polypeptide.

23. The method of claim 22 wherein the agent is a cellular receptor or a downstream effector.

24. A method for identifying an agent that modulates the expression or activity of the polypeptide of claim 1, the method comprising:
(a) providing a cell expressing said polypeptide;
(b) contacting the cell with said agent, and
(c) determining whether the agent modulates expression or activity of said polypeptide, whereby an alteration in expression or activity of said peptide indicates said agent modulates expression or activity of said polypeptide.

25. A method for modulating the activity of the polypeptide of claim 1, the method comprising contacting a cell sample expressing the polypeptide of said claim with a compound that binds to said polypeptide in an amount sufficient to modulate the activity of the polypeptide.

26. A method of treating or preventing a ENDOX-associated disorder, said method comprising administering to a subject in which such treatment or prevention is desired the polypeptide of claim 1 in an amount sufficient to treat or prevent said ENDOX-associated disorder in said subject.

27. The method of claim 26 wherein the disorder is selected from the group consisting of diabetes, metabolic disturbances associated with obesity, the metabolic syndrome X, anorexia, wasting disorders associated with chronic diseases, cancers, cancer-associated cachexia, and dyslipidemia.

28. The method of claim 26 wherein the disorder is related to organismal energy metabolism that effect adipose stores, muscle mass, insulin secretion, glucose utilization and serum lipid levels including triglycerides and cholesterol

29. The method of claim 26, wherein said subject is a human.

30. A method of treating or preventing a ENDOX-associated disorder, said method comprising administering to a subject in which such treatment or prevention is desired the nucleic acid of claim 5 in an amount sufficient to treat or prevent said ENDOX-associated disorder in said subject.

31. The method of claim 30 wherein the disorder is selected from the group consisting of diabetes, metabolic disturbances associated with obesity, the metabolic syndrome X, anorexia, wasting disorders associated with chronic diseases, cancers, cancer-associated cachexia, and dyslipidemia.

32. The method of claim 30 wherein the disorder is related to organismal energy metabolism that effects adipose stores, muscle mass, insulin secretion, glucose utilization and serum lipid levels including, triglycerides and cholesterol

32. The method of claim 30, wherein said subject is a human.

34. A method of treating or preventing a ENDOX-associated disorder, said method comprising administering to a subject in which such treatment or prevention is desired the antibody of claim 15 in an amount sufficient to treat or prevent said ENDOX-associated disorder in said subject

35. The method of claim 34 wherein the disorder is selected from the group consisting of diabetes, metabolic disturbances associated with obesity, the metabolic syndrome X, anorexia, wasting disorders associated with chronic diseases, cancers, cancer-associated cachexia, and dyslipidemia.

36. The method of claim 34 wherein the disorder is related to organismal energy metabolism that effects adipose stores, muscle mass, insulin secretion, glucose utilization and serum lipid levels including, triglycerides and cholesterol

37. The method of claim 34, wherein the subject is a human.

38. A pharmaceutical composition comprising the polypeptide of claim 1 and a pharmaceutically-acceptable carrier.

39. A pharmaceutical composition comprising the nucleic acid molecule of claim 5 and a pharmaceutically-acceptable carrier.

40. A pharmaceutical composition comprising the antibody of claim 15 and a pharmaceutically-acceptable carrier.

41. A kit comprising in one or more containers, the pharmaceutical composition of claim 38.

42. A kit comprising in one or more containers, the pharmaceutical composition of claim 39.

43. A kit comprising in one or more containers, the pharmaceutical composition of claim 40.

44. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease;
wherein an alteration in the expression level of the polypeptide in the first subject as compared to the control sample indicates the presence of or predisposition to said disease.

45. The method of claim 44 wherein the predisposition is to cancers.

46. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

47. The method of claim 46 wherein the predisposition is to cancers.

48. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NO:2, 4, 6, 8, 10, 15, 16, 17, 18, 19, 20, 21, 23, 24, 26, 27, 29, 30, 32, 33, 35 to 45 inclusive, 47, 49, 153, 157, 166, 175, 179, 183, 192, and 198, or a biologically active fragment thereof.

49. A method of treating a pathological state in a mammal, the method comprising administering to the mammal the antibody of claim 15 in an amount sufficient to alleviate the pathological state.
